(19) United States
(12) Patent Application Publication
(10) Pub. No.: US 2006/0206946 A1
(43) Pub. Date: Sep. 14, 2006

(54) Method of disrupting heme transport in nematodes and of modelling and evaluating eukaryotic heme transport

(75) Inventor: Iqbal Hamza, Kensington, MD (US)

Correspondence Address: LOWE HAUPTMAN BERNER, LLP, 1700 DIAGONAL ROAD, SUITE 300, ALEXANDRIA, VA, 22314, US

(73) Assignee: UNIVERSITY OF MARYLAND, COLLEGE PARK, College Park, MD (US)

(21) Appl. No.: 11/259,248

(22) Filed: Oct. 27, 2005

Related U.S. Application Data
(60) Provisional application No. 60/652,975, filed on Feb. 15, 2005.

(51) Int. Cl. A01K 67/033

(52) U.S. Cl. 800/3; 800/13

(57) ABSTRACT

A method for treating helminthic infections in a mammal or plant which entails administering one or more compounds which are metal-ligand chelate compounds containing a metal and a tetrapyrrole compound or a porphyrin compound, to mammal or plant in need thereof.

### FIELD OF THE INVENTION

The present invention relates to a method of disrupting heme transport in parasitic helminths, and a method of modelling and evaluating eukaryotic heme transport.

### DESCRIPTION OF THE BACKGROUND

Iron deficiency is the most common nutritional disorder. According to the World Health Organization, four out of five people in the world may be iron deficient, making nutritional iron deficiency one of the top ten risk factors in both developed and developing countries. See Micronutrient deficiencies. Battling iron deficiency anemia: The challenge 2003 http://www.who.int/nut/ida/htm. In developing countries, iron deficiency is multi-factorial due to dietary insufficiencies that are compounded by destruction of red cells from endemic malaria and intestinal bleeding because of parasitic hookworms. See Oppenheimer, S. J., J. Nutr. 131, 6165-6335 (2001). In the United States, iron deficiency is most prevalent among minority females and young children. Perinatal iron deficiency negatively impacts intelligence and congnition in children. See Gordan, Brain Dev. 25, 3-8 (2003).

Clearly, it is important to address iron deficiency, per se, rather than merely addressing diseases and conditions arising from underlying iron deficiencies. Such studies would provide novel insights into the interplay between genetics and nutrition in human populations, identify interacting nutrient deficiencies with other micronutrients such as copper or zinc, and aid in controlling disease susceptibilities.

Ironically, iron is one of the most abundant metals in the earth's crust, and it is plentiful in a variety of plants and seeds. Yet, iron deficiencies exist as much of the iron in the environment is not easily assimilated by mammals for essential metabolic processes. For example, iron in plants is not readily bioavailable to humans because plant-derived constitutents such as phytates interfere with its absorption across the intestine. By contrast, dietary heme is more easily absorbed than inorganic iron and is the source for two-thirds-of body iron in meat-eating individuals (from red-meat) even though heme constitutes only one-third of total dietary iron. See Uzel, C., Semin. Hematol. 35, 27-34 (1998). This is because heme is soluble at the pH of the intestine and its uptake is not influenced by dietary components that may affect the absorption of iron. Although it has been postulated that heme-iron is absorbed across the intestine by an active, energy-dependent and inducible process that may require a heme transporter identification of such a heme transport system has proved to be intractable due to lack of genetic and molecular tools to directly identify the genes involved.

Hemes are the prosthetic groups for many biological processes including oxidative metabolism, xenobiotic detoxification, synthesis and sensing of diatomic gases, cellular differentiation, gene regulation at the level of transcription, protein translation and targeting, and protein stability. See, Ponka, P. Am. J. Med. Sci., 318, 241-256 (1999). Within cells, protoheme (iron-protoporphyrin IX) is synthesized via a multistep biosynthetic pathway with well-defined intermediates that are highly conserved through evolution. Depending upon the organelle and cell type, heme pathway intermediates are utilized in the synthesis of other tetrapyrrole compounds including bilins, chlorophylls, and corrins.

The first universal precursor for the synthesis of heme is δ-aminolevulinic acid (δ-ALA). Heme synthesis culminates when ferrochelatase catalyzes insertion of ferrous iron into the protoporphyrin IX ring to form protoheme in the mitochrondria. See Lehninger, A., Biochemistry (Worth 1972). Protoheme is incorporated into numerous heme proteins or is modified further to synthesize other types of heme found in cytochrome c and terminal oxidases. Although hemes are found in all phyla, certain prokaryotic organisms such as Borrelia burgdoferi and Treponema pallidum neither make heme nor contain hemoproteins and the protozoa, Leishmania spp. appears to lack seven of the eight enzymes of the heme pathway. See, Sah., J. F. et al., J. Biol. Chem., 277, 14902-9 (2002). In these cases, the respective genomes reflect a lack of selective pressure to maintain the genes that were rendered non-essential by association with a eukaryotic host.

As observed with humans who absorb dietary heme as an iron source, some prokaryotes also utilize heme-iron living within the milieu of a eukaryotic host, where free iron is not readily available. In such microorganisms, the pathway for heme-iron acquisition and assimilation from heme-binding proteins such as hemoglobin, haptoglobin and hemopexin becomes essential for survival. In stark contrast to the lack of mechanistic insights on heme acquisition in eukaryotes, the mechanisms of heme uptake and processing in prokaryotes have been characterized at the genetic and biochemical level. See, Stojiljkovic, I. et al., DNA Cell Biol. 21, 281-295 (2002).

Helminthic infections are a serious burden to public health and global agriculture. See, for example, Science 293, 1437-1438 (2001). More than two billion people are infected by helminthiases and schistosomes, and plant-parasitic nematodes cause an estimated annual crop loss of eighty billion dollars. Clearly, there is an urgent need to find unique vulnerabilities in helminths because drug resistance by nematodes is already prevalent in livestock and other animals, and schistosomes resistant to praziquantel have been documented in places where this anti-helminthic drug is copiously used. Within a parasitized host, helminths exhibit distinct nutritional adaptations such that they acquire their food unidirectionally from the host to sustain their growth and reproduction. Thus, metabolic pathways essential for nutrient acquisition in worms could be exploited as potential drug targets to control helminthic infections.

Phylogenetic analysis of biosynthetic enzymes in the evolutionarily conserved multistep pathway for heme synthesis, δ-aminolevulinic acid dehydratases (ALAD) and porphobilinogen deaminases (PBGD), has suggested that C. elegans lacks orthologs for these enzymes and therefore may acquire tetrapyrroles nutritionally. See Jaffe, Chem. Biol., 10, 25-34 (2003). Correspondingly, the trypanosomatid protozoa, Leishmania spp. appears to lack seven of the eight enzymes of the heme pathway with the exception of ferrochelatase. This defect in tetrapyrrole synthesis is manifested as a nutritional requirement for heme or its immediate precursor protoporphyrin IX. Early studies demonstrated that Caenorhabditis elegans, Caenorhabditis briggsae and Rhabditis maupasi require cytochrome c or hemoglobin as a heme source for growth and reproduction. However, it is unclear why these nematodes require heme to grow and whether this nutritional necessity also exists in related helminths.

Further, despite extensive current knowledge of heme biosynthesis and the intermediates of this pathway in both prokaryotes and eukaryotes, the means by which heme is processed from the point of synthesis to its insertion into hemoproteins is unknown. Knowledge of eukaryotic heme transport mechanisms would, if known, allow for both the study and treatment of heme and iron deficiencies, for example, in humans with iron deficiencies and genetic mutations affecting heme synthesis. However, to date, knowledge regarding eukaryotic heme transport mechanisms beyond synthesiss is unavoidable.

### SUMMARY OF THE INVENTION

Accordingly, it is an object of the present invention to provide a model system for studying the mechanisms of eukaryotic heme transport downstream of origin.

It is also an object of the present invention to provide parasitic helminths, which are heme auxotrophs.

It is also an object of the present invention to provide a catalogue or library of mutants and alleles of C. elegans which may be used in studying mammalian heme transport mechanisms.

Moreover, it is also an object of the present invention to provide a method of treating a helminthic infection in a mammal, which entails administering to a mammal in need thereof an effective amount of one or more compounds which disrupt heme transport in the helminth infecting the mammal.

It is, moreover, an object of the present invention to provide a method of treating as well as preventing against helminthic infections in plants, which entails either treating a plant or soil in which the plant is located with one or more compounds which disrupt heme transport in the helminth.

It is, further, an object of the present invention to provide a model system and method for identifying a eukaryotic heme transport system.

### BRIEF DESCRIPTION OF THE DRAWINGS

FIG. 1A depicts a systematic model of heme homeostasis in eukaryotic cells with currently unknown heme pathways marked with a “?”.

FIG. 1B depicts heme transport through the apical intestinal surface in the nematode C. elegans

FIG. 2A depicts the ultrastructure of C. elegans polarized intestinal cell in an electron micrograph cross-section of a pair of worm intestinal cells.

FIG. 2B depicts a close-up of C. elegans microvilli on the apical surface of the intestinal cell shown in FIG. 2A with a human duodenal microvilli.

FIG. 3A is a reduced-minus-oxidized cytochrome absorption difference spectra of total extracts obtained from C. elegans wild-type strain N2 grown in defined CeHR media (containing 19 μM heme).

FIG. 3B is a Reduced-minus oxidized absorption spectra of pyridine hemochromes from either C. elegans mitochondrial and cytosolic fractions (scans 1 and 2), or from total extracts obtained from heme defective mutants of yeast (S. cerevisiae) and E. Coli (scans 3 and 4).

FIG. 4A shows the need of C. elegans for heme for growth and reproduction using synchronized L1 larvae as the primary noculum to analyze for aerobic growth in CeHR defined media.

FIG. 4B depicts a quantitative assessment of C. elegans growth in the presence of increasing amounts of hemin chloride.

FIG. 5A is an absorption spectra of pyridine hemochrome obtained from synthesized heme using protoporphyrin IX and ferrous chloride as substrates.

FIG. 5B is a Reduced-minus-oxidized absorption spectra of pyridine hemochromes extracted from intact N2 C. elegans with methylethyl ketone. Commercial hemin was used as a standard.

FIG. 5C depicts fluoresence determined in live worms by excitation of porphyrin in the FITC channel using a Leica Fluorescent Microscope fitted with CCD Digital Imaging (40×).

FIG. 6 depicts an overall scheme of a forward genetic screen to identify mutants.

FIG. 7 pertains to heme auxotrophy of worms, with Figures (A), (B), (C), (D) and (E) being described below.

(A) Dithionite-reduced minus ferricyanide-oxidized absorption spectra of pyridine hemochromes from total homogenate, membrane- and cytosolic-enriched fractions of C. elegans grown in axenic mCeHR medium supplemented with 20 μM hemin chloride. A peak at 557 nm and trough at 541 nm indicates pyridine protohemochrome. All samples were reduced with 5 mM sodium dithionite or oxidized with 1 mM potassium ferricyanide. The vertical bar represents a ΔA of 0.005 for total homogenate, 0.012 for membrane fraction and 0.02 for cytosolic fraction. Inset: Immunoblot of the same samples (50 μg) that were separated by 4-20% SDS/PAGE and probed with ATP2p antisera followed by chemiluminescent detection. This immunoblot was stripped to remove ATP2p antibodies and re-probed with alpha-tubulin antibody.

(B) Ultra low-temperature spectrum of whole homogenate from C. elegans grown in mCeHR medium supplemented with 20 μM hemin. Only alpha bands are indicated for cytochrome c, b and oxidase (a+a3). The vertical bar represents a ΔA of 1.0.

(C) Aerobic growth of C. elegans in mCeHR medium supplemented with 0.20 μM hemin chloride, or 20 μM protoporphyrin IX (disodium salt). Equal numbers of synchronized L1 larvae were used as primary inoculum in 24-well plates in triplicate and the cultures analyzed quantitatively for growth at days 1, 3 and 7.

(D) Biphasic response of C. elegans cultured in the presence of increasing amounts of hemin chloride (μM). Equal numbers of synchronized L1 larvae were grown in 24-well plates in mCeHR medium for 9 days and quantified (worms/μl) by microscopy. Each data point represents the mean±SD from three separate experiments performed in triplicate.

(E) Metabolic labeling in C. elegans cultured in the presence of heme. Synchronized L1 larvae were grown in mCeHR medium containing either ⁵⁹Fe or ⁵⁹Fe-heme (9.4×10⁶ DPM) and the worms harvested as gravid adults. Heme was extracted and concentrated, and then resolved by TLC followed by detection with a PhosphorImager (top panel). Lane 5, ⁵⁹Fe-heme control. Radiolabeled bands were quantified in a gamma counter and CPM normalized to total protein (bottom panel). To correct for non-specific binding of the radiolabeled Fe and heme, parallel experiments were conducted in the presence of 1 mM sodium azide (samples 1 and 3).

FIG. 8: Pertains to the characterization of heme uptake in C. elegans, with Figures (A), (B), (C) and (D) being described separately below.

(A) Aerobic growth of C. elegans in mCeFIR medium with 20 μM hemin supplemented with either gallium protoporphyrin IX (GaPP) or gallium salts. Synchronized L1 larvae were grown for 9 days in 24-well plates and quantified (worms/μl) by microscopy. Each data point represents the mean from a single experiment, and each experiment was performed in triplicate. Inset depicts the GaPP analysis at lower concentrations for clarity.

(B) Effect of heme on the cytotoxicity of GaPP. Synchronized L1 larvae were inoculated in 24-well plates containing mCeHR medium with either 0, 2, 4, or 6 μM GaPP and increasing hemin (μM). The number of worms per μl was measured on day 9 and the data are presented as mean±SD.

(C) Fluorescent metabolic labeling of worms with either 40 μM hemin (images 1, 4) or 40 μM ZnMP/4 μM hemin (images 2, 3, 5, 6) for 3 h followed by confocal microscopy with a 546 laser (images 1-3) and DIC optics (images 4-6). Arrowheads indicate ZnMP fluorescence accumulation within intestinal cells and developing embryos. For clarity, the boxed image in 2 is magnified in images 3 and 6. (Bar=100 μm).

(D) Worms were incubated with 40 μM ZnMP/4 μM hemin for 16 h followed by a chase with 40 μM hemin. Worms were analyzed by epifluorescence microscopy (TRITC channel) and DIC optics. Experiments were performed either in the absence (images 1-4) or presence (images 5-8) of NaN₃ during the chase periods to test for the non-specific loss of ZnMP fluorescence. Photomicrograph 4 is shown at a lower power to depict the complete loss of ZnMP fluorescence. (Bar=100 μm). For (C) and (D), four separate experiments were performed with a minimum of 50 worms per data point per experiment. The data are representative for >90% of worms analyzed.

FIG. 9 depicts worm utilization of heme-iron under iron deprivation. Equal numbers of synchronized L1 larvae were grown in the presence of 4, 20, and 100 μM hemin, either in basal mCeHR medium (set 1), or basal medium lacking exogenous iron (set 2), or as set 2 with 1 μM of the iron chelator ferrozine (set 3), or as set 3 with 486 μM ferrous ammonium sulfate (set 4). These values of ferrozine and iron were empirically determined by performing dose-response experiments and analyzing worm growth. The number of worms per μl was measured on day 9 and the data presented as mean±SD performed in triplicate. P<0.001 between sets 1 and 3. Within each set, values with different letters are significantly different. * denotes significant differences with the corresponding heme concentrations in set 1.

FIG. 10 is a phylogenetic maximum parsimony tree which shows that the loss of the heme pathway is common among free-living and parasitic nematodes.

FIG. 11 illustrates the overall scheme of Gene Chip analysis.

FIG. 12 illustrates an example of snip-SN (RFLP) mapping using bulked segregant analysis.

FIG. 13(A), (B), (C) and (D) illustrate heme-dependent growth phenotype of ten heme-resistant mutants.

### DETAILED DESCRIPTION OF THE PREFERRED EMBODIMENTS

The present invention is based, in part, upon the surprising discovery that C. elegans, for example, and other “medically relevant” helminths are heme auxotrophs. As used herein the term “medically relevant” helminths means these helminths which are parasitic to, or otherwise have an adverse effect on mammals or plants. In fact, their parasitic nature toward mammals, including humans, now appears to be a consequence of their heme auxotrophy. This discovery has numerous important aspects.

First, C. elegans, for example, may be advantageously used as a model system for studying the mechanisms of eukaryotic heme hemeostasis, i.e., downstream of heme synthesis.

Second, the present invention affords a catalogue or library of alleles and mutants of C. elegans that may be used in studying eukaryotic heme homeostatis.

Third, the present invention affords a method of treating helminthic infections in mammals, as well as compounds for effecting the treatment.

Fourth, the present invention provides a method of treating helminthic infections in plants as well as compounds for effecting the treatment.

Other advantageous aspects of the present invention will be described hereinafter.

In most free-living eukaryotes studied thus far, heme is synthesized from a series of intermediates through a well-defined, evolutionarily conserved pathway. Notably, glycine and succinyl CoA react to form enzyme-bound ∝-amino-β-ketoadipic acid, which then decarboxylates to yield δ-amino-levulinic acid. Two molecules of δ-amino-levulinic acid then condense to form porphobilinogen. Four molecules of porphobilinogen then serve as the precursurs of protoporphyrin. Iron is incorporated into protoporphyrin IX in the mitochondria. See Biochemistry, A. Lehninger (Worth 1972). Surprisingly, in accordance with the present invention, it has been discovered that free-living helminths, such as nematodes, for example, (or “worms” as is used hereinbelow), including the model genetic organism Caenorhabditis elegans, i.e. C. elegans, and other parasitic helminths are unable to synthesize heme de novo, even though these animals contain hemoproteins that function in key biological processes. Radioisotope, fluorescence labeling, and heme analog studies suggest that C. elegans acquires heme from exogenous sources. Iron-deprived worms were found unable to grow in the presence of adequate heme unless rescued by increasing heme levels in the growth medium. These data indicate that although worms utilize dietary heme for incorporation into hemoproteins, ingested heme is also used as an iron source when iron is limiting. The present invention provides a biochemical basis for the dependence of worm growth and development on heme, and also provides a model system for studying eukaryotic heme transport, whereby helminthic heme transport pathways can be preferentially targeted by pharmacological means for treating and controlling helminthic infections without adversely affecting the host heme transport system.

Heme (iron-protoporphyrin IX) is an important source of dietary iron for human nutrition. From a cellular perspective, hemes are synthesized within the eukaryotic mitochondria via a highly conserved, well-defined multistep pathway. It is presently unknown how heme is transported out of the mitochondria and incorporated into a vast number of hemoproteins including cytochrome P450s, peroxidases, calatases, hemoglobin and myoglobin. Excess heme, however, is toxic due to its inherent peroxidase activity. We assert herein that within all cells specific pathways exist for the efficient uptake, trafficking and sequestration of heme. Further, the molecules involved in these pathways can be identified using the model nematode Caenorhabditis elegans. Biochemical and sequence analysis has indicated that this nematode lacks the ability to synthesize heme de novo despite comprising all the essential hemoproteins, suggesting the presence of a robust heme transport system. Consistent with this observation, we have surprisingly found that nematodes have an absolute requirement for heme when grown in axenic synthetic helminths, such as media. Specifically, nematodes, such as C. elegans, do not have a heme biosynthetic pathway. Rather, since nematodes like C. elegans are bacteriovorous, they appear to acquire required heme as a nutrient from bacteria via the intestine, and then incorporate the ingested heme in toto into heme proteins. This now also appears to be the case generally, or parasitic nematodes, such as Ascaris suum, Trichuris suis, Haemonchus contortus, Strongyloides stercoralis, Ancylostoma duoclenale and species of Ancylostoma. We have determined, for example, that under normal conditions, C. elegans utilizes heme in toto and not as a source of iron, evidencing two separate pathways for heme and iron utilization. Interestingly, we have found that iron-deprived worms are able to utilize heme for growth, suggesting a specific mechanism for heme degradation that is induced when iron is limiting. The present invention is based, in part, on the discovery that C. elegans is a unique model system to elucidate the molecular pathways for eukaryotic heme homeostasis.

In eukaryotes heme is synthesized in the mitochondria. Yet, it is also important to know how heme is transported through the mitochondrial inner membrane to specific hemoproteins in the endoplasmic reticulum, cytoplasm, mitochondria, peroxisomes, and plasma membrane. It is also desirable to know the mechanisms for incorporating heme into the apo-proteins and if these mechanisms are specific to target apo-proteins and their sub-cellular milieu. Humans have abundant intracellular hemoproteins such as hemoglobin, myoglobin, and heme enzymes including 57 cytochrome P450s, 9-adenylate cyclases, soluble guanylate cyclases, peroxidases, catalases, and cytochrome oxidases. These enzymes are located in different cellular compartments and perform diverse functions depending upon heme as a prosthetic group. Free heme is hydrophobic and is insoluble in aqueous milieu. Hemes also have a potent peroxidase activity that easily damages biological macromolecules. Evaluation of the mechanistic pathways of the various hemoproteins in mammals particularly humans, is of great interest.

In accordance with one aspect of the present invention, a genetic and molecular approach is used to identify the mechanisms of eukaryotic heme homeostasis by utilizing the tractable and powerful genetics offered by the nematode Caenorhabditis elegans. As noted above, we have determined that C. elegans does not have a heme biosynthetic pathway but synthesizes a large number of heme proteins, which breaks the paradigm that heme synthesis occurs in all eukaryotic organisms. Because C. elegans is bacteriovorous, it appears to acquire heme as a nutrient from bacteria via the intestine and then incorporate the heme moiety in toto into hemoproteins. In principle, this mode of heme acquisition may be similar to dietary heme absorption by the human intestine. Since C. elegans lacks the ability to make heme, we are now able to also identify pathways that are downstream from the point of heme uptake—heme sequestration and trafficking. Thus, C. elegans provides an excellent model system and serve as a unique paradigm to define and identify the cellular transport and trafficking of heme in eukaryotes, such as mammals.

C. elegans has served as a model organism for defining biological processes for over forty years. The genome is sequenced, and a comprehensive, development cellular rate has been determined. See www.sanger.ac.uk/Projects/C_elegans/and also www.genome.wustl.edu/projects/celegans/. More than 70% of all human genes are conserved in C. elegans, and genes are identifiable by forward and reverse genetic screens. Moreover, the ability to grow this nematode in a controlled environment makes the organism ideal for micronutrient studies. Importantly, both the host (C. elegans) and its food (E. coli) are genetically tractable organisms, permitting the mechanism of nutrient uptake to be elucidated and evaluated. Yet surprisingly, the C. elegans model system has been largely unexploited for evaluating nutrient uptake and metal homeostasis despite the fact that it has a defined and highly versatile intestine for nutrient absorption. We have used this model system as a cornerstone to define the role of single nutrients in biological processes.

The implications of the present invention are far reaching. Identification of the eukaryotic, particularly mammalian, heme transporter allows for the design of more bioavailable forms of iron or prophyrin-based nutraceuticals to deliver iron more effectively to iron deficient populations. Like C. elegans, other related nematodes such as those noted above, and particularly intestinal hookworms, that aggravate iron deficiency are now, also implicated as heme auxotrophs. Elucidation of eukaryotic heme transport allows for the selective targeting of nematode heme transporters by rational drug and inhibitor design that can discriminate between helminthic versus human transporters. Identification of mechanisms for heme acquisition by cytochrome P450 proteins, the key drug and xenobiotic metabolizing enzyme in humans provides novel insights into how pharmaceuticals and toxins modulate biologic responses. Finally, characterizing how heme is transported in organisms affords knowledge of new pathways of intracellular heme trafficking that will parallel our ongoing work on copper chaperone pathways. Thus, the present invention also provides an important model for defining the role of specific nutrients in the etiology of human pathophysiology and malnutrition, which then provides for strategies to prevent and ameliorate the mortality and morbidity associated with a number of human diseases.

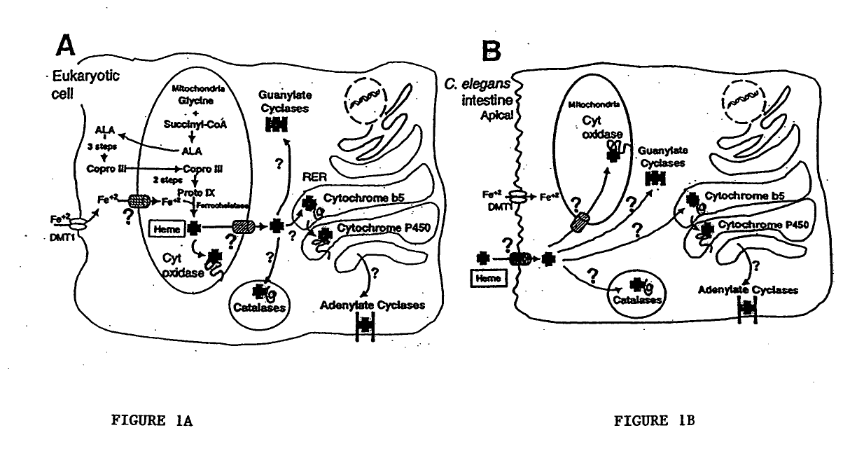

Current understanding of heme biosynthesis and its regulation, has made it possible to integrate many of the cellular pathways into a single model of heme homeostatis in the eukaryotic cell (FIG. 1A). Heme is synthesized via a defined multi-step pathway, shared between the cytosol and the mitochondria in eukaryotes. The pathway culminates with the insertion of iron into protoporphyrin IX ring catalyzed by ferrochelatase, a mitochondrial inner membrane-associated enzyme. Free heme is cytotoxic due to peroxidase activity, as are its two substrates; iron can generate hydroxyl radicals due to Fenton reaction, while protoporphyrin IX catalyzes light-dependent generation of oxygen radicals. Thus, heme biosynthesis is coupled with nutritious iron availability and with apo hemoprotein synthesis.

Despite research efforts from various groups utilizing microscopy, biochemical, and cell biological approaches to identify the pathways involved in heme transport, the precise mechanisms and molecules involved in transport of heme across biological membranes to cellular destinations have been elusive.

The present invention uses a genetic and biochemical approach to identify the mechanisms of eukaryotic heme acquisition and trafficking utilizing C. elegans as a model system. Database searches of all publicly available genomes were performed, and we have determined that the C. elegans genome has no orthologous genes to the eight highly conserved heme biosynthesis genes. This is, indeed, astonishing because the genome of this nematode contains abundant genes encoding for heme-binding proteins that have mammalian counterparts. We have now equivocally determined that C. elegans cannot synthesize heme de novo and is a heme auxotroph.

Our working model for heme homeostasis in C. elegans entails specific protein(s) that mediates transport of dietary heme from the intestinal apical surface (FIG. 1B). Once heme is translocated to the intracellular milieu, specific pathways involving hemochaperones appear to exist for trafficking of heme to distinct sub-cellular compartments for incorporation of heme into apo-proteins.

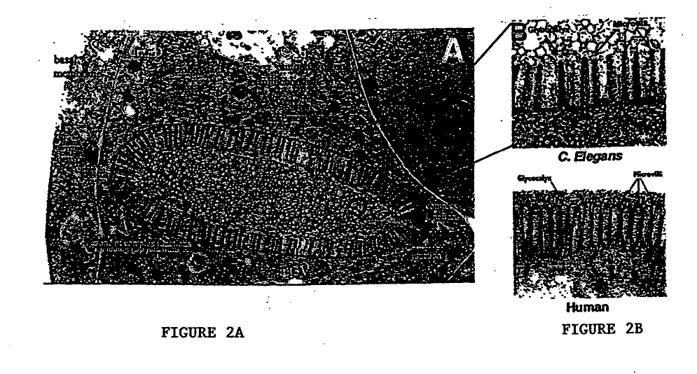

In C. elegans, uptake of ingested nutrients occurs at the level of intestinal absorption (FIGS. 2A and 2B). The digestive and metabolic activities of the intestine are central to the growth and development of the nematode. The primary function of the polarized intestinal cells are digestive since they secrete enzymes such as cysteine proteases and endodeoxyribonuclease into the lumen and absorb processed material and nutrients. The nematode intestine also appears to function as a storage organ since it contains a large number of assorted storage granules. The intestine performs multiple functions that are executed by organs other than intestines in mammals, e.g., fatty acid metabolism. Along the length of the intestine, the anterior and posterior parts of the intestine differ in certain functions. The anterior organ releases digestive enzymes, while the posterior portion contains yolk and lipid vacuoles and is active in nutrient and energy storage. C. elegans intestinal cells have been carefully examined at the microscopic and anatomic level, and the embryonic cell lineages that give rise to the adult worm intestine has been identified.

Our observation that C. elegans does not synthesize heme de novo, and requires hemoproteins for sustenance is unprecedented. To confirm this empirically, C. elegans wild-type N2 strain was grown aseptically in synthetic CeHR growth medium. CeHR medium was preferred because, it is truly axenic i.e., it does not contain any foreign organism thus minimizing ambiguity in interpreting results, and the nematodes grow to high densities making them ideal for biochemical analysis. Further, single dietary components can be fine-tuned for nutritional studies, and notably hemin chloride (15 μM) and cytochrome c (4 μM) are the sole source of heme thereby allowing complete control of heme levels in the growth medium.

All nematode strains were obtained from the Caenorhabditis Genetics Center. Synchronized L1 larvae were inoculated in CeHR medium and grown aerobically by gentle rotation at 20° C. for 4 to 5 days. Gravid adult worms were harvested and lysed with a French Pressure Cell to achieve >95% lysis as determined by microscopic examination. The lysates were centrifuged at 3000×g to remove debris and yield a total crude lysate, or further centrifuged at 9000×g to yield a pellet enriched in mitochondria (P2) and a post-mitochondrial supernatant (S2). Heme biosynthetic enzyme activities were measured in worm lysates for 6-aminolevulinic acid dehydratase (ALAD) and porphobilinogen deaminase (PBGD) (Table I, below). There was no detectable activity for either enzyme in C. elegans extracts compared to wild type E. coli extracts, used as a positive control source of both enzymes. E. coli strain RP523 lacking the ALAD gene was used as a negative control. Because organisms such as Haemophilus influenzae and some extremophile bacteria contain only part of the hemebiosynthetic pathway, we deemed it possible that worms too have retained the ability to synthesize heme, but by utilizing an intermediate of the heme pathway. We addressed this possibility by analyzing ferrochelatase activity, the final enzyme in the 8-step biosynthesis pathway. In eukaryotes, ferrochelatase is mitochondrial and is associated with the inner membrane. Therefore, we used the yeast Saccharomyces cerevisiae as a positive control because heme biosynthesis and regulation has been extensively studied in this eukaryote, and heme defective mutants are available commercially (Invitrogen and Open Biosystems). Ferrochelatase activities were undetectable in C. elegans and ferrochelatase mutant strain (hem 15) lysates compared to wild-type yeast (See Table I, below). This lack of activity was not because of differences in mitochondrial number or integrity during sample preparation as activity of another inner mitochondrial membrane protein, succinate dehydrogenase, was readily detectable.

TABLE I
Heme Biosynthetic Enzymes Activities

| | activity (Nmol/mg/min | | | | |
|---|---|---|---|---|---|
| | | E. coli* | | S. cerevisiae | |
| Enzymes Assayed | C. elegans | wild type | ALAD mutant | wild type | FC mutant |
| δ-Aminolevulinic Acid dehydrates | wild type | 11.43 ± 0.8 | 0.59 ± 0 | nd | nd |
| (ALAD) | | | | | |
| Porphobiliogen Deaminase (PBGD) | 0 | 0.124 ± 0.02 | nd | nd | nd |
| Ferrochelatase (FC) | 0§ | 16.36 ± 2.71 | nd | 4.1 ± 1.62§ | 0§ |
| Succinate Dehydrogenase | 725 ± 160§ | 1865 ± 318 | nd | 1545 ± 261§ | 915 ± 177§ |

Values (triplicates) nmol/mg/min of product formed are given as mean ± SD
nd: values not determined
*Crude extracts from wild type or heme biosynthetic mutants of E. coli and S. cerevisiae were used as controls
§Crude mitochondrial and cytosolic preps were isolated. Total activity is average of both preps.

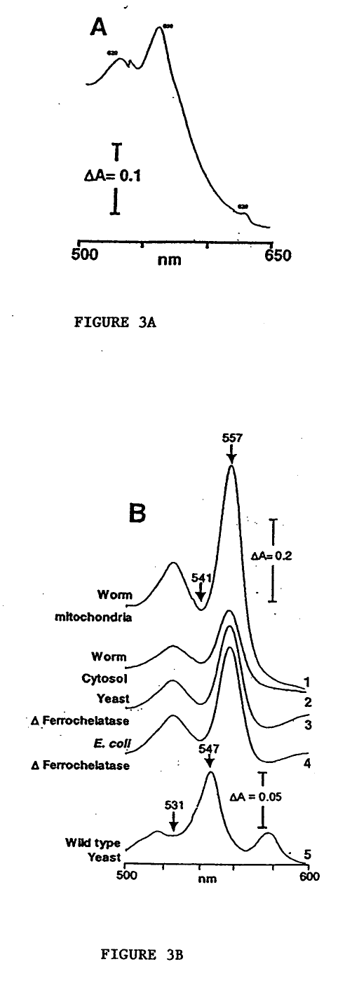

Cytochrome difference spectra of C. elegans extracts reveal that worms synthesize abundant hemoproteins as discerned by various cytochrome heme peaks (FIG. 3A). Because CeHR medium contains 19 μM heme, worms grown in this medium must utilize the supplemented heme to fulfill their heme requirement. In order to unequivocally establish that C. elegans is a heme auxotroph, we tested the ability of worms to catalyze the conversion of mesoprotoporphyrin IX to mesoheme. Heme auxotrophs are unable to catalyze this conversion because they lack ferrochelatase. That this was the case for extracts of C. elegans is summarized in FIG. 3B. Yeast and E. coli ferrochelatase mutants were used as controls, as these strains are unable to catalyze this conversion.

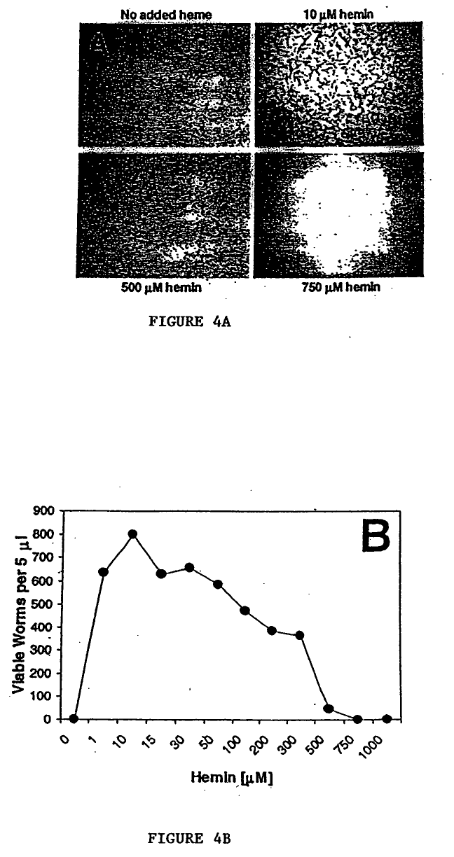

To qualitatively and quantitatively determine the heme requirement of C. elegans, we assessed growth at various heme concentrations. Equal numbers of L1 larvae were inoculated in CeHR medium with either no cytochrome c and hemin, or with cytochrome c and increasing amounts of hemin (FIG. 4A). The effect of hemin became apparent within 48 hours and by 9 days, the heme-deplete worms were severely growth retarded at the L1/L2 stages. Maximum growth was achieved at 10 μM hemin where L1 larvae grew to adults, laid large number of eggs, and propagated to multiple generations. However, this stimulatory effect of heme was toxic to worms grown at heme concentrations >750 μM. To quantitatively assess the effect of heme on worms, we counted the number of live worms grown under different concentration of heme using DIC microscopy. These measurements confirmed our qualitative analysis, and indicated that worms have a biphasic survival curve with respect to heme levels in the growth medium (FIG. 4B). On the basis of this association between heme and the growth and reproduction of C. elegans, we conclude that the biphasic nature of heme is likely due to the need for heme under heme-deprived conditions to perform essential metabolic functions, and the lethality of heme at high concentrations can be attributed to its cytotoxic activity.

We are now able to dissect the role of heme and hemoproteins in modulating biologic responses during normal growth and development in C. elegans, by defining the mechanism of heme acquisition, and identifying and characterizing-mutants with disruption of heme homeostasis. The heme dose-response curve has provided us with the upper and lower limits of heme requirement for C. elegans growth. This threshold range can now be used to conduct genetic screens for identification of worm mutants that can survive and grow under high and low heme concentrations, which would otherwise be lethal or inhibit growth in wild-type worms.

While it is apparent that heme is essential for the survival of organisms both as a prosthetic group and as a bioavailable form of iron, the unique aspect of utilizing the C. elegans genetic model is that this eukaryote has zero background noise. Thus, the results from our experiments are not confounded by endogenous heme synthesis, but reflect solely heme acquired from the diet. This allows us to make accurate quantative measurements of cellular heme status for biochemical analysis, and also augments the subsequent genetic characterization of interesting mutants with defects in heme uptake and utilization.

In addition to these unique and important features, three other effects are notable. First, abnormal heme acquisition in mutant worms is presumably much more severe than that observed in simple nutrient heme dose-response experiments. This degree of in vivo heme deficiency and toxicity cannot be reproducibly achieved by simple dietary manipulations. Second, heme deficiency is also compounded by the loss of activity of targets specific to heme trafficking. A severe defect in heme uptake, for instance, will disrupt all or most downstream activities including heme incorporation into multiple hemoproteins resulting in defects in enzymes such as CYP45O (daf-9) or cytochrome oxidase (cco). Third, as observed in bacteria, heme homeostasis may be globally regulated at the level of gene transcription in C. elegans, and mutations in this global regulator will lead to pleiotropic effects that are secondary to heme dependent pathways. Thus, we can determine the effects on C. elegans development of impairment in intracellular homeostasis. The striking heme-dependent growth phenotype presented evidences the strength of this approach. Altogether, the worm, in this case a nematode, model represents a unique opportunity to define in precise molecular terms the role of heme in animal development, and determine heme-specific targets in biochemical and genetic programs involving growth and development.

Although the pathways for heme transport and trafficking in eukaryotes were previously unknown, specific proteins and regulatory mechanisms have been described in bacteria that govern the acquisition of heme from the environment, including proteins that mediate heme insertion into specific hemoproteins such as cytochrome c. These studies indicate that heme, a cytotoxic molecule, cannot diff-use readily through lipid bilayers but is actively assimilated. By virtue of the present invention, we now provide a scheme for cellular heme homeostasis whereby heme is translocated across biological membranes in eukaryotes via specific transporters and subsequently trafficked to different cellular compartments by heme chaperones. Our studies with C. elegans indicate that this nematode is unique and provides an excellent eukaryotic paradigm to elucidate the mechanisms of heme assimilation.

Further, the present invention affords the characterization of the biochemical and cell biological mechanisms of heme acquisition by C. elegans, a natural heme auxotroph, with respect to time and heme concentration during stages of worm development. To better understand this pathway, it is imperative to first delineate the biochemical mechanisms of heme transport. We conduct these experiments with intact worms because primary cells derived from C. elegans are difficult to culture in vitro.

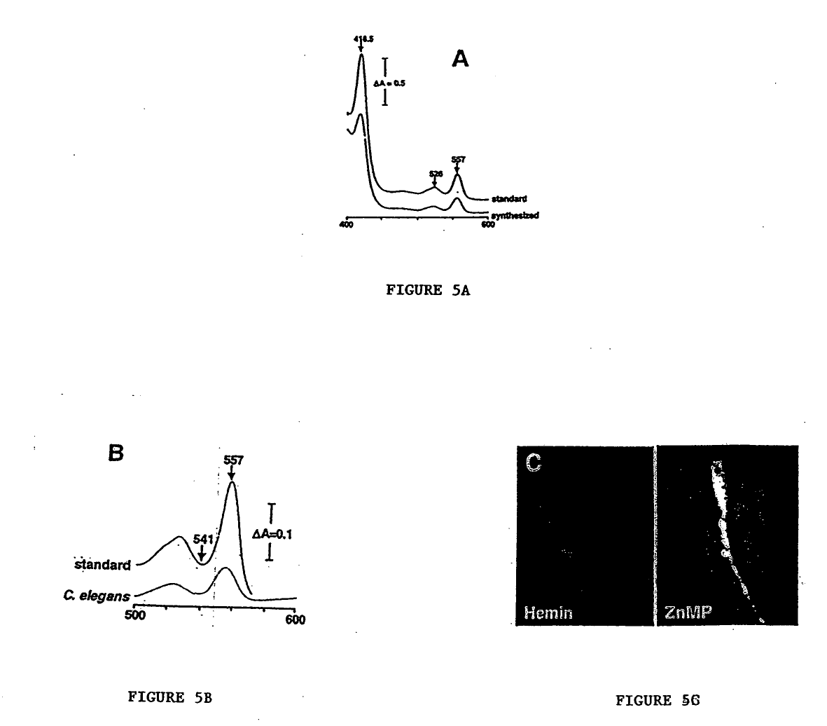

Heme levels in the growth medium have a dramatic effect on C. elegans growth, development, and reproduction. To test whether this effect is directly due to a concomitant change in the intracellular heme levels of the animal, we perform heme uptake studies with radiolabeled heme synthesized in our lab. ⁵⁹Fe is chemically inserted into protoporphyrin IX (PPIX) to synthesize heme. Various isotopes of iron may be obtained from Medical Isotopes, Inc., for example. However, this method is simple and does not rely on preexisting heme biosynthesis enzymes or intermediates in biological extracts. Two milliCurie (2.22×10⁹ dpm) of ⁵⁹Fe (FeCl₃, 35.77 mCi/mg) is purchased from Perkin Elmer, and porphyrin compounds are acquired from Frontier Sciences. PPIX, dissolved in pyridine, is added to glacial acetic acid at 50° C. and stirred under nitrogen. Vacuum distilled ⁵⁹Fe, dissolved in methanol and glacial acetic acid, is mixed with the PPIX maintaining stirring, gas flow and temperature for 1-2 h. The incorporation of ⁵⁹Fe into PPIX is monitored spectrophotometrically and will be complete when there is no further reduction in the absorbance of PPIX in pyridine at 408 nm. The mixture is dissolved in ethyl acetate and then washed extensively with HCl and water to remove unincorporated Fe and PPIX. Ethyl acetate is removed by rotary evaporation and the radiolabeled heme stored under vacuum till further use. We have practiced this methodology using unlabeled FeCl₃ and have achieved >70% efficiency in chemical synthesis of heme (FIG. 5A). Although we have ⁵⁹Fe as a metal in the chelate-complex with heme compounds, other metals may be used as discussed further below using a corresponding preparatory procedure with the appropriate, corresponding metal chloride, i.e., using Mn, Zn, Sn, Cu, Co, or Ga, for example, instead of Fe. The various metals which may be used in accordance with the present invention are discussed further below.

Synchronized L1 worms are inoculated in sterile T75 flasks containing CeHR medium, and worms will be harvested at L2, L3, L4 or gravid adult growth stages. Worms are counted by anesthetizing a small aliquot with 8% ethanol or 10 mM sodium azide in M9 buffer. Equal number of worms per sample are utilized for radiolabeling and growth curve experiments. Heme uptake and accumulation in cultured C. elegans is assayed by metabolic labeling with ⁵⁹Fe. Approximately 20,000 staged worms are plated in triplicate onto 24 well plates containing CeHR medium with no added hemin or cytochrome c. Uptake assays are initiated by incubating 10⁶ cpm of ⁵⁹Fe-heme for different time points at 20° C. by rotation. This method of direct metabolic labeling is more accurate and can be easily manipulated during kinetic analysis, compared to radiolabeling E. coli prior to feeding these bacteria to worms. Non-specific background is taken into account by performing a mock uptake with worms incubated at 4° C. or with potassium cyanide to inhibit metabolic processes. Initial heme uptake is measured at 0, 15, 30, 60 and 120 min. time intervals and with multiple heme concentrations (0.1 mM, 0.5 mM, 1 mM, 4 mM) utilizing ⁵⁹Fe-heme as a tracer. Accumulation studies are done by incubating each well of worms with 10⁵ cpm for 12, 24, 36 and 48 h time points with concentrations pre-determined from our kinetic analysis. For pulse-chase experiments, worms are heme-starved for 4-6 h to deplete endogenous heme levels, followed by pulse labeling with ⁵⁹FE (10⁵ cpm/well) for 14 h followed by several timed chase periods in CeHR medium containing molar excess of unlabeled “cold” heme. The precise times of incubation are optimized experimentally.

At the end of each experiment, worms are vacuum filtered through 0.45 μm cellulose acetate filters, washed copiously with cold M9 buffer/10 mM EDTA to remove nonspecific radiolabel, and lysed with cold M9 buffer/1% SDS/1% DOC/10 mM EDTA for 30 min on ice. Correction of the tissue content for radioactivity diffused into the extracellular space is performed by incubation with a nonpermeant carbohydrate used to calculate the relative size of the extracellular space in animal tissue during metabolite uptake assays. The specificity of heme uptake is determined by 50-100 fold molar excess of other tetrapyrroles including unlabeled heme, proto-, meso-, uro-, and copro-porphyrins. We determine whether heme uptake is energy dependent by analyzing uptake in the presence of metabolic inhibitors. This is accomplished by preincubating worms with antimycin A, oligomycin, sodium azide, ouabain, or carbonyl cyanide m-chlorophenylhydrazone (all from Sigma Chemicals) followed by a short pulse of ⁵⁹FE-heme. The concentration of and length of preincubations with these metabolic inhibitors is empirically determined. As a positive control for metabolite uptake and to test the efficacy of inhibitor treatments, the energy-dependent transport of [3H]succinate, a dicarboxylic acid known to be transported by NaDC2 gene product in the worm intestine, is measured, ⁵⁹Fe gamma radiation is measured in a Wallac 1470 Gamma Counter. Total protein is measured by the Bradford or bicinchoninic acid methods, and the data normalized to mol/mg of total protein or mol/number of worms. Prior to the start of each experiment, worm viability and morphology are monitored using DIC microscopy.

⁵⁹Fe-heme measurements are correlated with total heme content in whole worms by spectrophotometry. This is accomplished by acidification of worms with cold 0.1 N HCI followed by organic extraction of heme with ice-cold methylethylketone. The ketone is removed by rotary evaporation and the total heme (radiolabeled heme plus unlabeled heme) is dissolved in 1N NaOH for measurements. This method affords extraction of only the intact form of heme and not ⁵⁹Fe or PPIX, which remain in the acid phase. This protocol has been used successfully by us to obtain accurate estimates for the amount of heme present in intact worms (FIG. 5B). The heme samples obtained thus, are measured for total heme in a Shimadzu dual-beam scanning spectrophotometer and the percentage of radioactive heme is determined in a gamma counter. To ensure that ⁵⁹Fe-heme is not being degraded either spontaneously or enzymatically by a heme oxygenase-like enzyme we also perform uptake with ¹⁴C-heme, which has the radiolabel in the PPIX tetrapyrrole ring. In principle, the uptake of either isotope of heme should be identical. ⁵⁹Fe-heme studies are verified with ⁵⁹FeCl₃ uptake because even though the radioisotopes are identical, the compounds are different (⁵⁹FeCl₃ vs. ⁵⁹Fe-heme) and should have different kinetics.

Heme incorporation into hemoproteins is evaluated by analyzing ⁵⁹Fe signal utilizing Lithium dodecylsulfate/polyacrylamide gel electrophoresis (LDS/PAGE). LDS is used instead of SDS to prevent the loss of heme during electrophoresis. We have also had considerable success in radiolabeling proteins with ⁶⁴Cu using similar techniques. Synchronized worms that are metabolically labeled with ⁵⁹Fe-heme are harvested by centrifugation at 100×g and washed three times with M9 buffer 10 mM EDTA. These worms are then incubated in M9 buffer for 1-2 to empty their intestinal contents prior to lysis in 0.14 N NaCl/0.1 M Tris, pH 7.4 buffer with a French Pressure cell at 15,000 psi. We have standardized this lysis protocol to disrupt the worm cuticle and acquire homogenous worm suspensions that can be further fractionated using differential centrifugation to obtain sub-cellular organelles. We currently use a panel of 36 different antibodies (Sigma, Calbiochem, and BD Biosciences) directed against proteins from various organelles in mammals for cross-reactivity with worm or nematode homologues. We use these cross-reactive antibodies to authenticate the purity of our fractions by immunoblotting. Unboiled non-reducing solubilized membranes (200-500 μg) are separated on LDS/PAGE gels and exposed to a PhosphorImager (Molecular Dynamics) for detection of ⁵⁹Fe-hemoproteins. The radiolabel signal obtained from these samples is correlated with quantitative staining of the hemoproteins on the same gel using tetramethylbenzidine peroxidase method (6.3 mM TMBZ:0.25 M sodium acetate (3:7)/30 mM H₂O₂). If sensitivity becomes an issue, luminescence labeling with luminol (2 mM luminal/0.4 mM indophenol/12% H₂O₂ in PBS) may be used, which is up to 20 times more sensitive. The amount of specific hemoprotein on the gels is detected by autoradiography and quantified using ImageQuant v5.2 software. Using this method, we are able to accurately measure the percentage of hemoproteins radiolabeled by determining the ratio of total hemoprotein, determined by ketone extraction and histochemical staining, to the signal of ⁵⁹Fe-hemoproteins by gamma counting and Phosphorimage analysis.

Our metabolic studies also address the spatial location of heme with respect to time. From this, we consider, what cell types and sub-cellular compartment the heme is localized in, and how this relates to temporal kinetics. We also consider how the temporospatial pattern charges during worm development. Previous studies have employed zinc and tin mesoporphyrins (MP) as fluorescent heme analogs because, they are postulated to be transported by the same pathways as heme, and heme catabolism enzymes such as heme oxygenase, which releases iron from heme, is unable to degrade them permitting timed fluorescence studies. We also use this technique to perform live cell imaging in worms with ZnMP (and SnMP) to visually characterize heme transport using microscopy (FIG. 5C). This technique has been successfully used in human intestinal and liver cell lines to determine the kinetic and cellular transport of heme. Based upon the parameters obtained from our studies with radiolabeled heme, we incubate synchronized staged worms for various lengths of time with different concentrations of ZnMP. This is a useful procedure as ZnMP is most likely transported and utilized, by the same trafficking pathways as heme. Thereby, heme effectively competes with and quenches the ZnMP signal. We test the specificity of ZnMP uptake into worm cells by competition with 10, 100, 1000 fold excess of unlabeled heme. Fluorescence intensity is captured in the FITC channel with a CCD camera (Retiga) mounted on an inverted Leica DMIRE2 microscope. The images are quantified using SimplePCI v5.1 software (Compix, Inc) by measuring pixel density versus fluorescence intensity. Precautions are taken to ensure that this correlation is within the linear range and multiple samples will be used per experiment for reproducibility.

These biochemical studies not only are important for understanding how C. elegans maintains heme homeostasis, but also allow us to isolate and characterize heme mutants. Even if the results from our biochemical measurements in whole worms do not reflect what may occur in single cells (eg: intestinal cells) because of limitations in the available methodologies, this does not affect our ability to characterize heme mutants. Further, where radiolabel heme experiments may not correlate with the ZnMP/SnMP fluorescence imaging approach either due to intrinsic differences between these compounds, or due to the very different approaches—biochemical versus cell biological, it is acceptable to directly visualize heme in worm sections by ultrastructural cytochemistry and autoradiography using electron microscopy using known methodologies. In fact, this approach has been used by others to pinpoint the precise location of vesicular heme in intestinal cells.

Identification and Characterization of C. elegans Mutants with Disruption in Heme Homeostasis

We describe herein the elucidation of the genetic specification of heme homeostasis in C. elegans, by performing a forward genetic screen and isolating mutants with defects in heme transport and assimilation. We also utilize parallel approaches to identify candidate genes involved in heme transport in C. elegans. Given the severe growth and developmental arrest observed in worms or nematodes under low and high heme conditions, genetic analysis can be conducted in an unbiased manner, by generating and screening for mutants with aberrant responses to heme. In addition, data obtained establishes the biochemical parameters and defines the threshold requirements for heme, thus allowing for an efficient screening strategy to dissect the genetic determinants in heme utilization.

A unique aspect of our methodology of genetic analysis is that we utilize sterile axenic CeHR liquid medium for the initial screen instead of petri dishes plated with E. coli on NGM agar. This affords several key advantages: (a) we perform saturation screens several times the worm or nematode genome without being labor-intensive, (b) our F2 screen is robust and eliminates “noise” from other mutant worms, because it is based on positive selection i.e., high heme severely affects worm growth and is toxic, (c) we can easily alter our approach e.g., screen for dominant mutations by exposing the F1 progeny to heme, or perform a F2 screen under low heme and identify animals that survive, (a) a single T75 flask easily accommodates 10⁶ worms conferring a higher likelihood of obtaining low-penetrance mutations, and (e) the effect of heme on worms grown in axenic liquid cultures is direct, rather than relying on E. coli to first assimilate heme; worms are 10 times more sensitive to heme when grown in liquid medium than on heme plates. Altogether, these salient features allow for the generation of a comprehensive catalogue of interesting mutants and alleles with specific defects not only in heme uptake, but also in heme trafficking and incorporation.

In order to further describe the present invention, reference will now be made to certain Examples which are provided solely for purposes of illustration and which are not intended to be limitative.

### EXAMPLE 1

Synchronized wild-type N2 worms (˜300 late L4 larvae), grown aseptically in CeHR growth medium, are mutagenized with 50 mM ethyl methanesulfonic acid (EMS) (Sigma) for 4 h at 20° C. EMS is used because of its proven mutagenic ability, although, based upon our positive heme-based selection, a recently described transposon-based mutagenesis may also be used. The worms are washed three times with sterile M9 buffer and allowed to recover in CeHR medium at 15° C. for 12-15 h. Worms are analyzed microscopically to ensure normal morphology, and 30 mutagenized worms (P₀S) will be transferred to each of the 8 separate 175 flasks (30×8=240 P₀S) and allowed to lay eggs at 20° C. The worms are carefully monitored every day to check for viability and allowed go through two generations to yield L1 larvae in the F2 progeny (about 8-9 days). This yields approximately 300,000 F2 worms in each flask (30 P₀×100 F1×100 F2=300,000) with ˜25% or 75,000 worms homozygous (m/m) for a mutation. Assuming there are ˜19,000 genes in C. elegans, it is possible to sample 48,000 haploid genomes or 2.5 times the entire worm genome (30 P₀×˜100 F1×8=˜24,000 F1 diploid genomes×2=˜48,000).

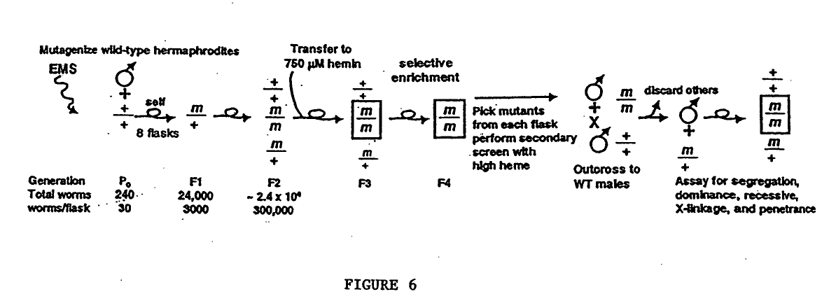

To prevent over sampling the genome of every F1 mutant, only half the contents of each flask (150,000 worms containing ˜37,000 m/m) are transferred to a new T75 flask containing CeHR medium supplemented with 750 μM hemin (FIG. 6). Results obtained indicate that 750 μM hemin results in growth arrest and lethality in wild-type worms (FIG. 4A). Thus, the basis of the initial or first screen is to identify mutants that survive and show normal growth and reproduction under high heme. F2 worms thus obtained are grown for two additional generations (F4). This secondary screen results in lowering the number of false positives, eliminating sterile worms (this may be as high as 30% for EMS), and selectively enriching the number of mutants that survive heme toxicity.

Although, a large number of mutant worms, is screened, a handful of mutant worms survives heme toxicity. Surviving mutant worms are classified from each of the 8 flasks as 8 separate classes of mutants. A sample of mutant worms (up to 10) from each flask is sub-cultured to ensure that the mutant phenotype breeds true. These mutants are then singled out and a battery of biochemical studies is conducted in liquid culture and on petri plates to characterize the defect in these animals with respect to heme dependent pathways. Each of these mutants is compared based on the following tests:

(a) detailed microscopic examination to test whether mutations in the heme pathway affect worm morphology and whether these differences can be phenotypically clustered.

(b) metabolic labeling with ⁵⁹Fe-heme to determine uptake, accumulation, effiux, and hemoprotein activity. These measurements provide a quantitative analysis of specific defects in either the transport, sequestration, detoxification, or incorporation of heme.

(c) comparison of ⁵⁹Fe-heme uptake and accumulation results with [³H]succinate uptake as a control for nutrient absorption in the gut. Mutants with general defects, due to gross changes in gut morphology or global changes in translation or transcriptional control of genes involved in nutrient absorption, reveal defects in both heme and succinate uptake. These mutants are discarded.

(d) live cell imaging of worms with ZnMP to visually characterize the defects in heme transport using fluorescent microscopy. These studies provide detailed insights into the cell biological defects in heme pathways i.e., decreased transport will result in lower fluorescence or aberrant trafficking and sequestration may reveal mislocalization of heme within cells.

(e) spectral analysis (as in FIG. 3A) of hemoproteins obtained by cell lysate fractionation to qualitatively measure the type of hemoprotein affected by the mutation e.g: cytochrome c versus cytochrome b or cytochrome P450. These studies provide a window into heme trafficking i.e., defects specific to a single class of hemoprotein(s).

(f) sensitivity to metal-ligand compounds, such as gallium protoporphyrin IX (GaPPIX). GaPPIX is heme analog, which we tested; worms were 30 times more sensitive to GaPPIX compared to heme. GaPPIX appears to gain entry into cells through the heme transport system and is incorporated into hemoproteins by heme trafficking pathways. This results in worms that have non-functional hemoproteins. This heightened sensitivity towards GaPPIX is exploited by testing our mutants for their ability to survive in a dose-response curve. It also provides a basis for treating helminthic infections of mammals and of plants, which will be described further below, including metal-ligand compounds to effect these treatments.

Initially, the mutants are tested by culturing them under low heme. Because the mutants are resistant to high heme toxicity, they may have mutations in a heme transporter such that less of the “toxic” heme is now transported into cells. In that instance, these mutants grow poorly compared to wild-type worms when challenged with low heme levels.

These studies are not performed sequentially, but rather simultaneously, with the 8 putative classes of mutants. Initially, attention is focused on mutants that reveal an overt phenotype with respect to heme entry into cells, because this step will be upstream of all, subsequent pathways. It is possible that loss-of-function mutation in an essential heme transporter may be embryonically lethal. However, mutations in specific regions of this protein may result in decreased activity of the transporter/receptor due to diminished affinity for binding of heme or a secondary molecule involved in the pathway. As stated earlier, the screen is easily modified to look for dominant gain-of-function mutations in a F1 (or F2) screen.

Depending on the number of mutants obtained in the F2 screen, parallel genetic complementation analysis is conducted to test whether the mutations are within the same gene (allelic) or different gene (non allelic). Males from mutant hermaphrodites are generated either by heat shock from incubating plates at 30° C. for 5 h, or by mating mutant L4 virgin hermaphrodites to wild-type males (as in our outcross, see FIG. 6). These males are used to carry mutations between mutant strains for complementation analysis. Based upon this analysis, we can classify whether the mutant genetic screen was saturated and identify the number of alleles and complementation groups obtained. Taken together with the biochemical and cell biological characterization, a comprehensive analysis of mutants with specific defects in heme homeostasis is provided. Some are depicted in Table II below. We have included potential mutants that can be obtained by screening for mutants that survive under low heme. These mutants may have complementary genes or alleles that are up regulated eg: increased function of a heme transporter.

TABLE II
Putative classes of mutants that may survive
heme toxicity or heme depletion

| High heme selection | Low heme selection |
|---|---|
| ↓ Heme transport/absorption | ↑ Heme transport/absorption |
| ↓ Heme trafficking downstream from | ↑ Heme trafficking downstream |
| transporter | from transporter, “faster flux” |
| ↑ Efflux of heme from cells | ↓ Efflux of heme from cells |
| ↑ detoxification of heme by increased | ↑ retention and sequestration |
| Peroxidase and CYP450 activity | of heme |
| ↑ storage/sequestration of heme | ↑ mobilization of heme from heme |
| | storage proteins |
| ↑ repressor or ↓ activator | ↓ repressor or ↑ activator |
| (transcriptional) | (transcriptional) |
| ↓ of entire trafficking pathway due to | ↑ of entire trafficking pathway due |
| specific heme-dependent- or | to specific heme-dependent- or |
| non-specific translational defect | non-specific translational defect |
| ↓ non-specific defect in nutrient | ↑ non-specific defect in higher |
| uptake due to morphological changes | nutrient uptake due to changes in |
| in pharyngeal pumping, gut | gut absorption |
| absorption, and defecation rates | ↓ general metabolism |

↑ = increased function,
↓ = decreased function

Based upon the nature of the complementation groups, the mutations are mapped and localized using standard techniques known from C. elegans genetics. The mutation is mapped to a chromosome by using mapping strains, MT465 [dpy-5(e61)I; bli(e768)II; unc-32(e189)III] and MT464 [unc-5(e53)IV; dpy-11(e224) V; lon-2(e678)X], obtained from GCG. These strains have three homozygous recessive mutations in each of the six chromosomes (I, II, III and IV, V, X). Mating mutant males to these mapping strains and scoring F2 progeny that segregate the heme dependent mutant phenotype with these markers afford information about the chromosomal location of the mutation. Two approaches are then used to fine-map the mutation: mapping to an interval using three-factor mapping, and restriction fragment length polymorphism in combination with single nucleotide polymorphisms (snip-SNPS).

The three-factor mapping depends upon the results from the experiment with the chromosome mapping strains described above. Mapping strains with three markers on a single chromosome are used to perform mating with mutant males, and the F2 progeny that segregate the mutant phenotype with these markers is scored. Repeating this analysis with other marker strains on the same chromosome provides the relative position of the mutation with respect to the chromosomal markers. For snip-SNPs we use the Hawaiian strain CB4856 which shows a high level of polymorpism across the genome compared to the wild-type N2 strain. Mutant homozygous hermaphrodites are crossed with CB4866 males to obtain hermaphrodite outcrosses that are heterozygous for the mutation. These animals are then allowed to self-fertilize to yield F2 progeny. Homozygous mutant worms are N2 for the genomic region surrounding the mutation, but are otherwise a mixture of both N2 and CB4856 genomes. This feature is exploited to perform “bulked segregant analysis” with separately pooled mutant and wild-type F2 worms. Repeated PCR analysis followed by digestion with specific restriction enzymes affords identification of the approximate location of mutant genes. Information regarding the coordinates of SNPs is found publicly at http://genome.wustl.edu/projects/celegans.

Finally, a detailed analysis of the mutated genes obtained from our mutant screen may be conducted. Confirmation and identification of the mutated genes may be conducted with known RNA interference techniques to scan genomic DNA contigs within the mutated regions, and complementation analyses with wild-type DNA to rescue the mutant phenotypes.

### EXAMPLE 2

As shown in FIG. 12, the basis of our F2 genetic screen was to identify mutants that survive and show normal growth and reproduction under high heme, because hemin concentrations of ≧800 μM results in growth arrest and lethality of wild-type worms (See FIG. 15). Synchronized wild-type N2 worms (˜6000 late L4 larvae), grown aspetically in CeHR growth medium, were mutagenized with 50 mM ethyl methanesulfonic acid (EMS) for 4 h at 20° C. (22). The worms were then washed three times with sterile M9 buffer and allowed to recover in CeHR medium at 15° C. for 12-15 h. These P₀ worms were analyzed microscopically to ensure normal morphology, and 3000 mutagenized P₀ worms were transferred to 10 separate T75 flasks (300×10=3000 P₀) and allowed to lay eggs at 20° C. The worms were checked for viability and allowed to go through one generation to yield gravid F1 adults that are heterozygous for a mutation. The F1 gravid hermaphrodite worms were treated with bleach (1.1% bleach/0.55 M NaOH) which liverates the bleach-resistant eggs from the worm carcasses. The eggs were extensively washed and hatched overnight in M9 buffer for growth synchronization. L1 larvae in the F2 generation obtained thus were exposed to selection of 800 μM hemin. Our objective here was to (a) quickly eliminate background (we now had 3×10⁶ F2 worms/flask) and sterile mutants, and (b) ensure that larvae at the L1 stage were exposed to high hemin because this developmental stage was most sensitive to heme-induced toxicity. Assuming there are ˜20,778 predicted genes in C. elegans (www.wormbase.org, Release 26.116a.1, Last Update: Aug. 2, 2004), we sampled 600,000 haploid genomes or 29 times the entire worm genome (300 P₀×˜100 F1×10 flasks=300,000 F1 diploids×2=600,000 haploid genomes).

To further increase the stringency of our genetic screen we split the contents from each 800 μM hemin selection flasks into two flasks containing either 800 μM or 1000 μM hemin. This allowed us to use another tier of positive selection to identify mutants with greater resistance to heme toxicity. Mutants from each flask were isolated and treated as a separate entity by giving an identification number based on the Genetic Nomenclature Guidelines used for C. elegans (http://biosci.umn.edu/CGC/Nomenclature/nomenguid.htm). To eliminate EMS-induced spurious mutations that could result in false positive, the mutants were outcrossed to wild-type N2 males. After five outcrosses, which replaces ˜97% of the mutant genome, mutants that bred true were selected for further analysis. Using this procedure, we isolated 13 individual mutants that were resistant to heme toxicity. These mutants were further characterized by analyzing Mendelian segregation ratios, X-linkage, phenotype penetrance, generation times, brood size, and their ability to grow under low hemin. All of the mutants isolated were recessive and showed complete penetrance under high heme. Although the mutants were selected for resistance to high heme toxicity, they showed an unexpected range of growth even under low heme (1.5 μM) (FIG. 13). This observation was surprising because we anticipated that high heme-resistant mutants will grow poorly under low heme. Based on the mutant's growth profile at low and high heme levels, we broadly categorized them into three phenotypic classes: class A, class B and class C (FIG. 13). These “phenoclusters” have the following growth phenotype: (a) three mutants IH728, IH938, IH731 show with robust growth under low and high (≧800 μM heme (FIG. 13B), (b) one mutant IH828 show exceptional growth under low heme, moderate growth at ≦800 μM heme, and no growth at 1000 μM hemin (FIG. 13C), and (c) six mutants IH921, IH11058, IH1068n, IH1068d, IH1048, and IH718 that show moderate growth under high heme (≦800 μM), but normal growth under low heme (FIG. 13D). By classifying these mutants into phenoclusters, we can focus our efforts on the phenotypic datasets to map the molecular networks and pathways involved in heme dependent processes. For instance, mutants in a cluster may have mutations within the same gene or in multiple genes but within the same pathway.

The heme dose-response experiment reveals a biphasic response to heme by C. elegans. Because of the nature of the growth curve, we reasoned that heme homeostatis in C. elegans may be regulated in accordance with organismic needs and metabolic demands. To test this hypothesis, worms were grown at 1.5, 4, 20, 100, 500, 800 and 1000 μM hemin CeHR medium. After one generation of growth, total worms were harvested by centrifugation at 100×g, washed in M9 buffer, and incubated in M9 buffer for 1 h to empty their intestinal contents. They were then pulse-labelled with 40 μM ZnMP for 3 h. Parallel experiments were also performed with worms anethetized in the presence of 1 mM sodium azide to account for non-specific binding of fluourescence to the samples.

C. elegans grown at ≦20 μM hemin revealed a robust uptake of flourescent ZnMP, compared to the dramatic decline in fluorescence in worms grown at ≧100 μM hemin. Our results suggest that heme uptake is regulated; high heme negatively regulates heme uptake while low heme positively stimulates heme transport and accumulation. This conclusion is physiologically reasonable because heme homeostatis should constitute a balance between the essential necessity for nutritious heme versus the cytotoxicity due to heme overload. These results, however, do not indicate whether heme regulation occurs at the transcriptional or post-transcriptional level. As a first step towards understanding heme homeostatis at the molecular level, we carried-out a genome-wide analysis using the Affymetrix C. elegans Genome Array to identify genes that are transcriptionally regulated by heme. See FIG. 11.

Worms were grown at 4 μM (low) 20 μM (optimal) and 500 μM (high) hemin in axenic CeHR medium. We chose 4 and 500 μM hemin because these heme concentrations were at either end of the worm growth spectrum, and worms at these heme concentrations show a ˜24 h growth delay compared to worms grown at 20 μM hemin. Despite this delay in maturation, the worms were at all three heme concentrations were morphologically indistinguishable and do not represent morbid animals. Importantly, the data also suggest that low and high heme exerts a physiologic effect on facet(s) of C. elegans growth and development. In order to eliminate maternal effect genes, wild-type N2 worms were grown for two synchronized generations in their respective hemin concentrations to obtain F2 worms at the late L4 stage as determined by morphological analysis of the vulva. F2 worms from all three heme conditions were harvested at the same mid-L4 stage for final RNA extraction. The RNA was extracted with Trizol and treated with RNase-free DNase to remove any contaminating DNA followed by a purification on a Qiagen RNeasy column. Each experiment was performed four independent times on four separate days to ensure proper sampling and to account for experimental variations. The final RNA samples were sent to the DNA microarray facility at NIH-NIDDK directed by Dr. Maggie Cam. A total of nine Affymetrix C. elegans genome arrays were used—three per experiment (4.20, 500 μM samples×3=9).

As depicted in FIG. 11, ˜4.26% of the worm genome corresponding to 886 genes showed changes in heme expression at either 4 or 500 μM hemin compared to 20 μM hemin samples used as baseline. From these genes, 58 genes were excluded because they were specific to germline, sperm or oocyte development, as determined by cross-checking with publicly available microarray databases. Using a threshold value of >2.0 fold-change in gene expression, we found 124 genes that showed significant regulation by heme in C. elegans. Changing this threshold to ≧1.6, provided an additional 156 genes. Notably, >150 genes had obvious human orthologs (E-value≧10⁻⁴), and >90% of the heme-regulated benes had no ascribed functions in C. elegans database, even though these genes encoded proteins with putative domains for transmembrane regions, nucleic acid binding, metallo-cofactor binding and transporters. Taken together, our initial results from the microarrays indicate that ˜1.35% of the genes in the worm genome (280 genes/20,778 total genes) responds to heme at the transcriptional level.

Materials and Experimental Procedures

Worm Culture and Growth Assays. Free-living worms were cultured in CeHR axenic liquid medium (Dr. Eric Clegg, Personal Communication). Worm growth rates (3.5 days), mobility, and development in CeHR medium were comparable to those cultured on E. coli in agar plates. CeHR medium was modified (henceforth called mCeHR) to eliminate all sources of exogenously added heme; basal growth medium comprised 20 μM hemin chloride (Frontier Scientific, Logan, Utah) and 150 μM of ferrous ammonium sulfate (Sigma Chemicals). Worm strains were grown in mCeHR medium under aerobic conditions in tissue culture flasks at 20° C. ˜3.7×10⁶ worms were routinely obtained after 3.5 to 4 days in a T75 flask with 30 ml basal medium from an initial inoculum of −1.5×10⁵ worms. For initial culturing of worms in axenic media, three generations of worms were sequentially bleached (1.1% bleach/0.55 M NaOH) to eliminate any contaminating bacterial carryover from agar plates. In all dose-response experiments, worms were growth synchronized by treating the gravid hermaphrodite worms with bleach to dissolve adult worms. The eggs, resistant to bleach, were liberated from the carcasses and extensively washed with M9 buffer followed by overnight hatching in M9 buffer to synchronize growth. By harvesting at appropriate time intervals, synchronous larval stages and adult staged worms were collected for experimental manipulations. Identical numbers of synchronized L1 larvae were inoculated into media with different heme concentrations in 12- or 24-well culture plates. Worm growth was monitored each day and an aliquot was obtained for counting by microscopy usually at days 3, 6, and 9. The worms in each well were counted twice and each growth condition was analyzed in triplicate. P values for statistical significance were calculated utilizing a one-way ANOVA with Student-Newman-Keuls multiple comparison test using GraphPad Instat version 3.01.

Preparation of hemin, hemin analogs and [⁵⁹Fe]Heme. Fresh stock solutions of hemin or hemin analogs (Frontier Sciences, Logan, Utah) were prepared immediately prior to use by dissolving required amounts in 0.3 M ammonium hydroxide. The pH of the stock solution was adjusted to pH 8.0 with 6 N HCl, and filter sterilized (0.45 μM). The upper limit of free iron was estimated to be 3.8 nM/1 μM of hemin chloride by inductively coupled plasma-mass spectrometry (ICP-MS) analysis. For synthesis of ⁵⁹Fe-heme, 50 ml of glacial acetic acid was stirred under a constant flow of N₂ at 60° C. followed by addition of 12 mg of protoporphyrin IX in pyridine for 30 min. To this mixture, 0.85 μCi of FeCl₃ (specific activity 35.77 mCi/mg, Perkin Elmer, Boston, Mass.) was stirred in for an additional 3 h. Heme was extracted from this mixture with ethyl acetate followed by extensive washes with 4 N HCl and distilled water to remove unincorporated protoporphyrin IX and iron. The heme thus obtained was concentrated by evaporation of the ethyl acetate using a RotaVapor and frozen at −20° C. until further use. Total amount of ⁵⁹Fe-heme synthesized by this method was measured using a Packard Gamma Counter (˜21% efficiency). The purity of heme was determined by thin layer chromatography using silica gel 60 matrix in an NH₄OH-saturated chamber with 2,6-lutidine/water solvent. We estimated the specific activity of ⁵⁹Fe-heme synthesized to be ˜2.8×10⁶ DPM/nmol.

Metabolic Labeling, Heme Isolation and Thin Layer Chromatography. Equal numbers of L1 worms were grown at 20° C. in mCeHR medium with 20 μM hemin and supplemented with 9.4×10⁶ DPM of either ⁵⁹Fe-heme or ⁵⁹FeCl₃. Radiolabeled adult worms were harvested by first incubating them in M9 buffer for 30 min to empty the gut contents. Worms were then extensively washed with large amounts of M9 buffer/lmMEDTA on a Gelman Metricel membrane (0.45 μM) which had been preincubated with 20 μM hemin or FeCl₃ to prevent non-specific absorption of the radiotracer signal to the filter membrane. The experiment was done in parallel in the presence of 1 mM sodium azide (NaN₃) to account for non-specific binding of the radiolabel to the biological samples. It was experimentally determined that this concentration of NaN₃ was not lethal to worms. To isolate heme, worms were washed with M9 buffer and resuspended in ice-cold 1 N HCl to a final pH of 2.0. The acidified solutions were incubated on ice for 30 min. to allow complete protein denaturation, and then an equal volume of ice-cold 2-butanone was added. The solutions were shaken and allowed to stand until the ketone (heme) and aqueous (worm debris) phases separated. The upper ketonic phase was removed and the heme concentrated by rotary evaporation. The heme was resuspended in dimethyl formamide and equal volumes of samples and controls were spotted and resolved on Silica gel 60 TLC plates. Plates were exposed to a PhosphorImager and the radiolabel signal corresponding to heme was excised and analyzed using a gamma counter. Counts (DPM) obtained were normalized for protein as determined by the bicinchoninic acid method (Sigma) performed on pre-aliquoted samples of intact worms. The specific activity of ⁵⁹Fe-heme added to the growth medium was 4.7×10⁴ DPM/nmol and the ⁵⁹Fe-heme extracted from worms was 0.69×10⁴ DPM/nmol. The most plausible explanation for this difference in specific activity of ⁵⁹Fe-heme is dilution of the supplied radiolabel heme with pre-existing unlabeled heme endogenous to the worm.

Pulse-Chase Analysis. Mixed populations of worms grown in mCeHR medium with 4 μM hemin were labeled for 16 h in the presence of either 40 μM zinc mesoporphyrin (ZnMP) or hemin. It was empirically determined that 40 μM ZnMIP labels the worms without affecting viability. Fluorescently labeled worms were washed with M9 buffer, dispensed into 12-well plates and allowed to incorporate unlabeled hemin at concentrations of 40, 400 or 800 μM. At timed intervals, aliquots of worms were removed into the appropriate medium containing 10 mM NaN₃ mounted on a slide and immediately analyzed with a 543 He/Ne laser on a Zeiss 410 confocal microscope, or with a Peltier-cooled Retiga 1300 12-bit CCD camera fitted on to a Leica DMIRE2 autofluorescence/DIC microscope. Images were further analyzed with SIMPLEPCI v5.2 Software (Compix, Inc.). Sensor gain and exposure times were kept constant during all image acquisition. No loss of fluorescence was observed when labeling experiments were performed in parallel in the presence of 1 mM NaN₃ during the chase period and when worms were incubated in medium without hemin. To account for background autouluorescence in C. elegans, the sensor gain of the CCD camera or the laser was set to subtract any fluorescence obtained from control worms incubated in the presence of 40 μM hemin.

Experimental Results

Analysis of publicly available worm genome databases revealed that these genomes lack obvious orthologs to heme biosynthesis pathway enzymes. Genome databases were querried by using sequences of the human enzymes which catalyze the seven sequential steps to synthesize heme from the first universal precursor δ-ALA. Expect (E) threshold values obtained from BLAST searches revealed non-significant hits only in the C. elegans database as compared to genome databases from S. cerevisiae, D. melanogaster, and mouse, thus suggesting that C. elegans lacks orthologous genes for enzymes that catalyze heme synthesis. These in silico observations were confirmed by measuring enzyme activity. Because free-living worms in the laboratory use E. coli as food, bacterial metabolites could confound identification of the source of heme and enzyme activity. Therefore, worms were grown axenically in mCeHR liquid medium in lieu of growth on Petri plates containing E. coli (see methods above). Three physiologically distinct but phylogenetically related free-living bacteriovorous strains, C. elegans, Panagrellus redivivus, and Oscheius myriophila were used. See Table III below. Synchronized worms were grown aerobically at 20° C. in mCeHR to the gravid adult stage and homogenized to obtain cytosol- and mitochondria-enriched fractions for analysis of heme biosynthesis enzymes. Under the assay conditions used, ALAD and PBGD activities were undetectable in worm lysates as compared to wild-type E. coli lysates.

TABLE III
Enzyme activity (nmol/mg/min)*

| Organism | Host | ALAD | PBGD | FC | SDH |
|---|---|---|---|---|---|
| Helminth | | | | | |
| Caenorhabditis elegans^{α} | Free | ND | ND | ND^{§} | 725 ± 160^{§} |
| Panagrellus redivivus^{α} | Free | ND | ND | ND^{§} | 3419.3 ± 424.7^{§} |
| Oscheius myriophila^{α} | Free | ND | ND | ND^{§} | 1567 ± 594.6^{§} |
| Paragordius varius | Free^{†} | ND | ND | (—) | 715.9 ± 9.2 |
| Schistosoma mansoni | human | ND | ND | ND^{§} | 515 ± 80.6 |
| Strongyloides stercoralis | human | ND | ND | ND^{§} | 715.2 ± 53.8^{§} |
| Ancylostoma caninum | dog | ND | ND | ND^{§} | 266.7 ± 45.6^{§} |
| Haemonchus contortus | sheep/goat | ND | ND | ND^{§} | 30.3 ± 4.6^{§} |
| Trichuris suis | pig | ND | ND | ND^{§} | 7.6 ± 0.8^{§} |
| Ascaris suum | pig | ND | ND | ND^{§} | 24.2 ± 3.2^{§} |
| Bacteria | | | | | |
| E. coli | n/a | 14.7 ± 0.03 | 0.035 ± 0 | 16.36 ± 2.71 | 1865 ± 318 |
| E. coli (ALAD mutant) | n/a | 0.034 ± 0 | (—) | (—) | (—) |
| Yeast | | | | | |
| S. cerevisiae | n/a | 0.97 ± 0.4 | 0.096 ± 0 | 27.42 ± 2.92^{§} | 2371.2 ± 32.1^{§} |
| S. cerevisiae (FC mutant) | n/a | 0.04 ± 0 | 0.032 ± 0 | ND^{§} | 915 ± 177^{§} |

*Mean values (triplicates) of product formed ± SD.
^{α}Grown in axenic mCeHR medium.
^{†}P. varius adults live in fresh water but larvae develop in arthropods.
^{§}Average activity found in combined crude mitochondria- and cytosol-enriched fractions.
ND: Enzyme activity not detected under current assay conditions.
n/a: not applicable.
(—): not assayed.
ALAD—δ-Aminolevulinic acid dehydratase,
PBGD—Porphobilinogen deaminase,
FC—Ferrochelatase,
SDH—Succinate dehydrogenase

The protozoan Leishmania and certain microorganisms such as Haemophilus influenzae contain only part of the heme biosynthetic pathway. Thus, it is possible that worms also have retained the ability to synthesize heme by utilizing an intermediate of the heme pathway. To directly address this issue, ferrochelatase activity was measured, an inner mitochondrial membrane enzyme, which catalyzes the final step in the heme biosynthetic pathway. Because ferrochelatase from S. cerevisiae has been genetically and biochemically well-characterized, yeast was used as a control source of this enzyme. Ferrochelatase activity was readily detected in wild-type yeast, but was undetectable in combined mitochondria- and cytosol-enriched fractions from all three worm species and a S. cerevisiae ferrochelatase mutant with genetic disruption at the HEM15 locus. Activity for succinate dehydrogenase, another inner mitochondrial membrane enzyme, was readily detectable in these fractions indicating the presence of mitochondrial membranes. However, the inability to detect heme synthesis enzyme activities could be attributed to the presence of endogenous inhibitors in worm extracts. No inhibition of ALAD, PBGD or ferrochelatase enzyme activities when was found C. elegans extracts were mixed in equal proportions with extracts from either E. coli or S. cerevisiae (See Table IV below).

TABLE IV

| | Enzyme activity (nmol/mg/min)* | | |
|---|---|---|---|
| Organism | ALAD | PBGD | FC |
| C. elegans | ND | ND | ND |
| E. coli | 1.35 ± 0.044 | 0.032 ± 0.002 | 9.52 ± 0 |
| S. cerevisiae | 0.69 ± 0.012 | 0.026 ± 0.002 | 10.59 ± 0.65 |
| S. cerevisiae FC | (—) | (—) | ND |
| mutant | | | |
| C. elegans + E. coli^{†} | 0.699 ± 0.05 | 0.018 ± 0.002 | 3.95 ± 0.37 |
| C. elegans + | 0.313 ± 0.001 | 0.015 ± 0.001 | 7.45 ± 0.76 |
| S. cerevisiae^{†} | | | |
| C. elegans + | (—) | (—) | (—) |
| S. cerevisiae | | | |
| FC mutant^{†} | | | |

*Mean values (triplicates) of product formed ± SD
^{†}Equal proportions (50%) of protein from each organism were mixed prior to assaying for enzymes. Because enzyme activity was normalized to total protein, activities are reduced by half.
ND: Enzyme activity not detected under assay conditions,
(—): not assayed
ALAD—δ-Aminolevulinic acid dehydratase,
PBGD—Porphobilinogen deaminase,
FC—Ferrochelatase.

To address whether the lack of discernable heme enzyme activities also held for other worm species that are phylogenetically diverse, five parasitic nematodes were examined that have different host specificities (Strongyloides stercoralis, Ancylostoma caninum, Haemonchus contortus, Trichuris suis, and Ascaris suum), a nematomorph (Paragordius varius), and a trematode flatworm (Schistosoma mansoni). Irrespective of their host affiliations, enzyme activities were undetectable in all helminthic extracts in our assay conditions. These measurements provide further support that parasitic helminths, as evidenced by those examined in this study, do not have enzymes for heme synthesis.

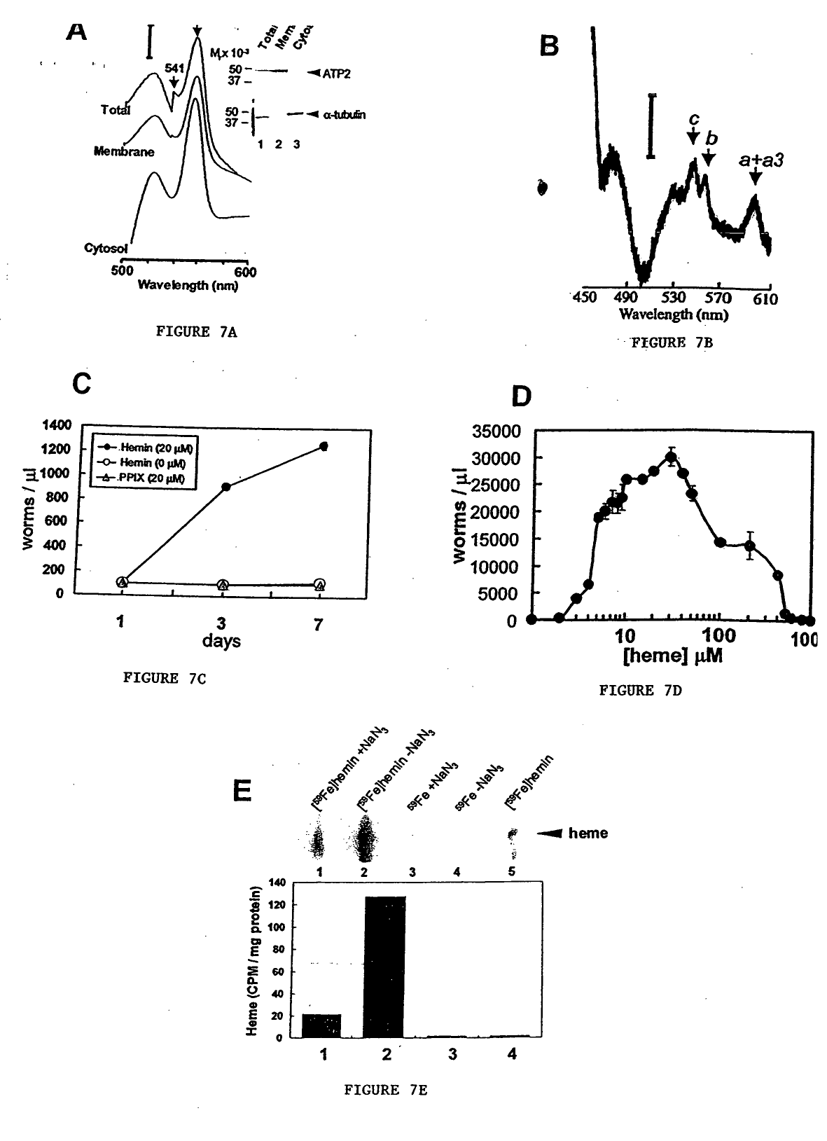

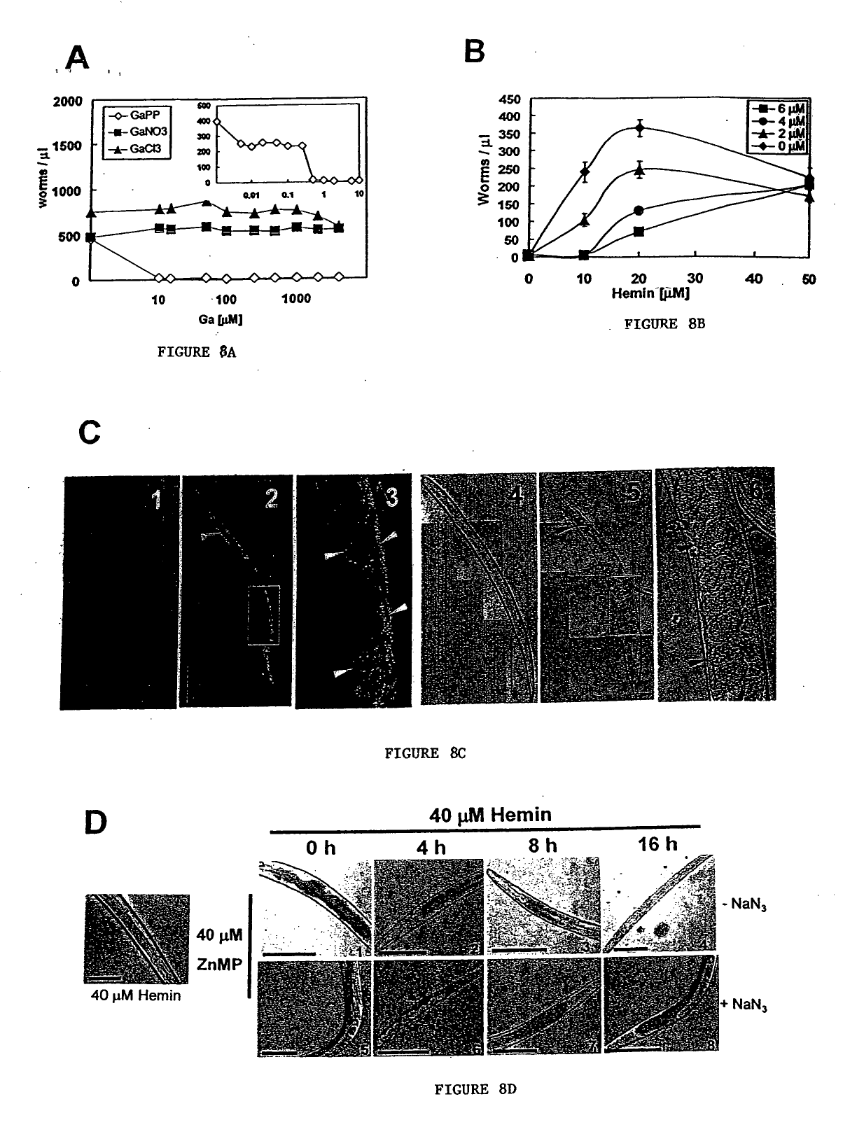

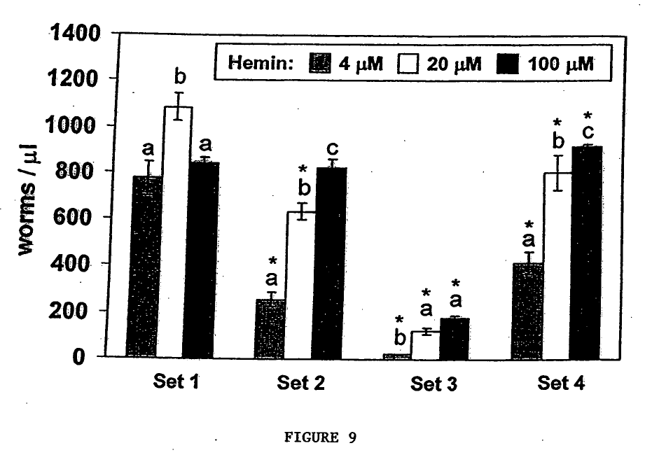

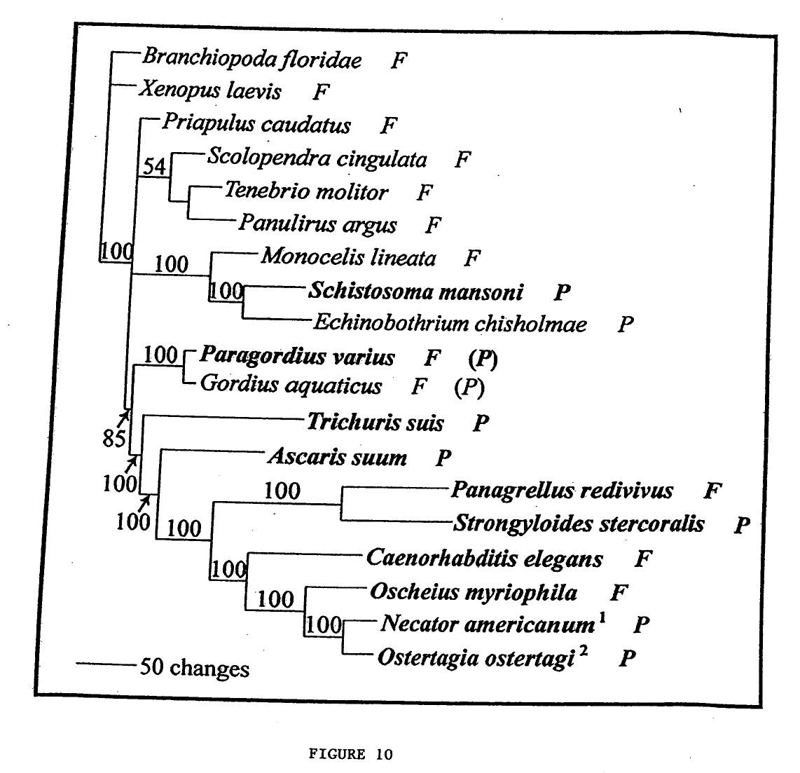

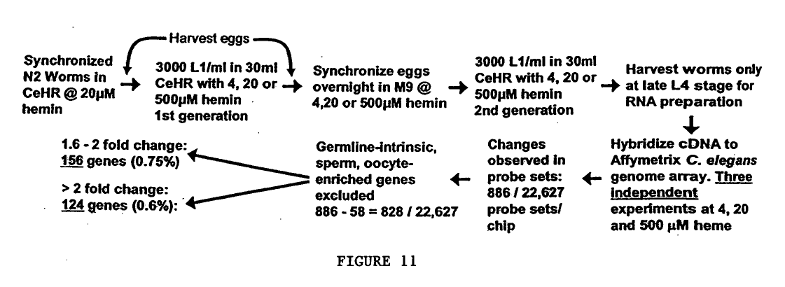

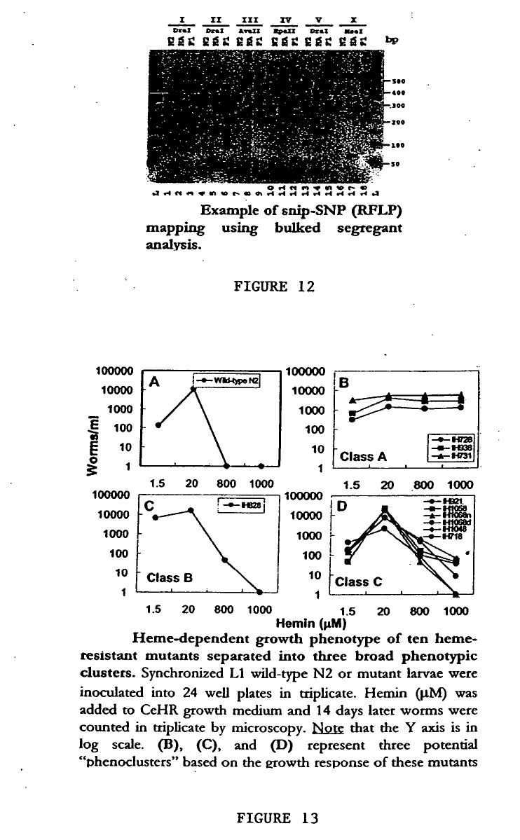

C. elegans appears to have a large number of hemoprotein orthologs, including globin isoforms, guanylate cyclases, adenylate cyclases, catalases, cytochrome P450s, and respiratory cytochromes. Although hemes have been found in all phyla, certain microbial pathogens such as Borrelia burgdorferi and Treponema pallidum neither make heme nor contain hemoproteins. Reduced minus oxidized absorption spectra of pyridine hemochromes revealed that C. elegans has discernable hemoproteins in worm fractions enriched either for cytosol or for membranes, including mitochondrial membranes (FIG. 7A). The purity of these fractions was determined by iminunoblots probed with antisera for α-tubulin and ATP2, the β-subunit of the F1 sector of the mitochondrial F₁F_{O} ATP synthase. Using ATP2 as a marker less than 1% of the signal was found in the cytosol-enriched fraction, while ˜88% of ATP2 protein was found in the membrane-enriched fraction. Ultra low-temperature spectra of total worm homogenates revealed the presence of detectable amounts of respiratory cytochromes a, b and c (FIG. 7B).

To quantitatively determine the heme requirement of worms, C. elegans was cultured in the presence or absence of nutritional heme supplements. Worms grown in the absence of exogenous heme (supplemented as hemin chloride) were growth arrested at the L4 stage, whereas worms grown in the presence of heme grew robustly and reproduced over multiple generations (FIG. 7C). Similar heme-dependent growth was also observed for P. redivivus and O. myriophila (data not shown). This lack of growth in the absence of heme was reversible, as replenishing heme to the heme-depleted media resulted in normal growth rates of the arrested larvae (data not shown). However, unlike Leishmania, which can exogenously acquire either hemin or its immediate precursor protoporphyrin IX for growth, C. elegans was unable to utilize protoporphyrin IX (supplemented as free acid or disodium salt) as a substitute for hemin (FIG. 7C). Cytochrome c or hemoglobin also sustained worm growth, supporting previous studies that have shown heme and vitamin B 12, another tetrapyrrole, as necessary factors for C. briggsae development. Our observations that worms can develop to the L4 stage in the absence of exogenous heme in the growth medium suggest that either there are trace amounts of heme in the medium, or more plausibly, maternal heme stored in the egg during embiyogenesis is able to sustain early larval growth.

Worms responded in a biphasic manner to heme when grown in the presence of various amounts of hemin (FIG. 7D). Although the optimum concentration of hemin for growth was 20 μM, worms continued to grow and reproduce at concentrations ranging from 1.5 μM to 500 μM hemin, albeit with significantly slower generation times and brood size. Large amounts of hemin resulted (≧800 μM) in growth arrest at the L3 stage possibly because hemes are cytotoxic due to peroxidase activity. To determine if C. elegans acquires heme directly from the growth medium, worms were metabolically labeled in the presence of equivalent amounts of ⁵⁹Fe or ⁵⁹Fe-heme or and worm homogenates analyzed for the respective radiotracer. A specific signal was obtained in samples containing worms labeled with ⁵⁹Fe-heme (FIG. 7E, lane 2). Thus, worms utilize ⁵⁹Fe-heme directly from the growth medium to fulfill their heme auxotrophy and incorporate the tetrapyrrole into proteins. However, no radiolabeled signal was observed in heme extracted from worms when ⁵⁹Fe by itself was used (FIG. 7E, lane 4). This observation corroborates our genomic and biochemical analysis (Table III) that C. elegans lack ferrochelatase, the terminal enzyme in heme biosynthesis. ⁵⁹Fe-heme uptake was negligible in worms that were metabolically inhibited in the presence of NaN₃ an inhibitor of the mitochondrial respiratory chain.

Studies with bacterial mutants that utilize heme and hemoglobin as an iron source have shown that non-iron metalloporphyrins act as heme analogs and gain entry into cells via high-affinity heme transport systems. Following uptake, non-iron metalloporphyrins show varying degrees of antibacterial activity depending on their metal cofactor. In order to determine whether a heme uptake system exists in C. elegans, synchronized worms were grown in the presence of 4 μM hemin and varying amounts of non-iron metalloporphyrins. We tested six different non-iron metalloporphyrins and found that gallium protoporphyrin IX (GaPP) was by far the most toxic heme analog. Compared to hemin, worms (P0) were growth retarded in the presence of 1 μM GaPP (800 fold sensitivity) (FIG. 8A), while F1 progeny, obtained from surviving P0 worms grown at lower concentrations of GaPP, were growth arrested at 0.25 μM GaPP (3,200 fold sensitivity). The ionic radii of Ga and Fe are very similar (0.62 versus 0.64). Because Ga cannot undergo oxidation-reduction reactions like Fe, organisms that have heme uptake systems probably utilize and incorporate GaPP as a cofactor instead of heme, resulting in cytotoxicity. Neither gallium chloride nor gallium nitrate was able to mimic the toxicity of GaPP (even at concentrations >100-fold) suggesting that the anti-helminthic activity of GaPP was not due to the adventitious release of Ga from GaPP (FIG. 8A). Notably, exposure of worms to GaPP resulted in developmental abnormalities that correlated in their severity to the levels of GaPP in the growth medium. These morphological phenotypes may reflect incremental inhibition of cellular pathways that are dependent on hemoproteins. Indeed, the toxic effect of GaPP was attenuated in the presence of increasing levels of hemin, indicating that GaPP exerts its anti-helminthic effect via heme-dependent pathways (FIG. 8B).

To further elucidate heme uptake at the cellular level, we utilized metabolic labeling in live worms with zinc mesoporphyrin IX (ZnMP). This fluorescent heme analog is not a substrate for heme catabolic enzymes such as heme oxygenases (HOs), and thus a fluorescent signal can be amplified over time as ZnMP accumulates in cells. Live worm imaging revealed a time-dependent accumulation of ZnMP in worms; fluorescence was detected in worm intestinal cells within 10 min. of treatment with 40 μM ZnMP. Confocal microscopy showed ZnMP accumulation in multiple cell types in the adult worm including the intestinal cells, eggs and dividing embryos (FIG. 8C). We found discernable fluorescence signal in embryos within 130 min. of incubation with ZnMP. This time point is likely an overestimate because ZnMP fluorescence is weak and is detectable only after a substantial signal has accumulated over time. To correlate the specificity of ZnMP fluorescence with heme transport, worms were first fluorescently labeled by feeding them 40 μM ZnMP. They were then washed to remove ZnMP and incubated with 40 μM hemin. The fluorescence intensity diminished over time and was undetectable by 16 h (FIG. 8D). This loss in fluorescence was specific, as the signal did not diminish when worms were treated in parallel with a non-lethal dose of sodium azide.

Bacteria and C. albicans that have high-affinity heme uptake systems utilize heme as an iron source when iron is limiting during infection within the host milieu. Heme oxygenase (HO) degrades heme to release iron, biliverdin and carbon monoxide; in some organisms, including mammals and cyanobacteria, biliverdin is subsequently converted to bilirubin by biliverdin reductase. Because the metabolism of heme and iron is interlinked, we determined how they are interrelated in worms. We grew C. elegans in either iron deplete or replete medium supplemented with low (4 μM), optimal (20 μM) or high hemin (100 μM). Worm growth was significantly slow in medium with 4 μM hemin but lacking exogenous iron (FIG. 9, sets 1 and 2). This diminution in growth was further accentuated in the presence of ferrozine, a membrane-impermeable iron chelator (FIG. 9, sets 3). Conversely, iron supplementation reversed the effect of ferrozine on worm growth (FIG. 9, sets 3 versus 4), indicating that worms need both heme and iron to sustain growth and reproduction under nutrient-sufficient conditions. Importantly, 20 and 100 μM hemin significantly (P<0.001) attenuated the growth retarding effects of iron deprivation even in the presence of ferrozine (FIG. 9, set 3). In the absence of iron, hemin concentrations are greater than 100 μM in the growth medium and did not result in any additional worm growth plausibly because heme toxicity could mask the beneficial effects of heme as an iron source. This growth-promoting effect of hemin in the absence of iron was not due to trace amounts of free-iron from hemin, because analysis of iron-dependent growth indicated that the amount of free-iron in hemin (3.8 nM/μM) is insufficient to support worm growth.

Analysis and Discussion

We have shown, using C. elegans as a model system, that helminths are exceptional among known free-living eukaryotes because they lack the ability to synthesize heme. This conclusion is supported by genomic, biochemical and nutritional analysis and by previous studies which show that heme is a growth factor for C. elegans development. This inability to make heme is surprising, given that other free-living metazoans make endogenous heme, and heme synthesis is catalyzed by enzymes encoded by at least seven separate genes that are not clustered in the genome. It is plausible that the ancestral worm lost the genes responsible for heme biosynthesis due to a lack of selective pressure because the progenitor had access to heme either from a parasitized host, or a symbiotic relationship with another organism. Recent studies have shown that the cattle tick Boophilus microplus, a blood-sucking arthropod relies on a blood meal to acquire heme, and pathogenic human filarial nematodes as well as certain insects harbor the bacteria Wolbachia, an intracellular symbiont that has a mutualistic relationship with its host, such that the nematode acquires endobacterial-derived metabolites. Indeed, the Wolbachia genome contains orthologs of genes for heme biosynthetic enzymes suggesting that this endosymbiont has the ability to make heme.

The phylogenetic maximum parsimony tree (FIG. 10) shows that the loss of the heme pathway is common rather than exceptional among free-living and parasitic nematodes, and can be found in higher taxa such as the Nematomorpha and Platyhelminthes, both of which have the potential for free-living and parasitic habits. While the closest outgroup to nematodes is still controversial, if one accepts the Ecdysozoa theory where arthropods are a sister group to nematodes, the loss of the heme synthesis pathway would have occurred more frequently than with a more traditional grouping of helminths. A loss in the heme pathway would be consistent with a close phylogenetic relationship among helminths. Additional taxon sampling along with genomic and biochemical analyses may help clarify the disputed phylogenetic placement of helminths in the animal kingdom. Evaluation of the complexity and phylogenetic importance of different molecular phylogenies versus the loss of the heme synthesis pathway cannot be made here. However, it is interesting to note that some other biochemical pathways also show distinct differences between classically simpler invertebrates (flatworms and nematodes) and higher invertebrates (arthropods and molluscs). These include an increased variety of different neuropeptides in higher invertebrates compared to lower and the absence of a Toll-like pathway for immunity in nematodes that is present in insects.

The present invention evidences that adaptation to heme auxotrophy in worms has enabled worms to utilize heme in its entirety as a prosthetic group under normal conditions and as an iron source when iron is low in the environment. We have been unable to identify any significant orthologs of HOs in the worm genome, although we found two putative orthologs of biliverdin reductase in the C. elegans and C. briggsae genomes. Because HOs from bacteria to man are a diverse group of heme-catabolizing enzymes, it is possible that heme degradation in C. elegans is either catalyzed by a HO with low sequence homology to known HOs or an entirely novel enzyme. Enzyme activities for HO and biliverdin reductase in homogenates from the trematode Schistosoma japonicum have been reported, thus raising the possibility that C. elegans may also have the ability to enzymatically degrade heme to obtain iron.

Because worms or helminths, for example, nematodes, lack the ability to make heme and therefore solely rely on exogenous heme for metabolic processes, these animals must have evolved specific mechanisms for intestinal heme absorption, trafficking, and sequestration. Perhaps, exceptional to worms, an intercellular heme transport system may be required to provide heme to other cell types beyond intestinal cells. Free heme is hydrophobic and is insoluble in the aqueous cellular milieu, and hemes have peroxidase activity that can damage biological macromolecules. Thus, in principle, intracellular pathways must exist for heme trafficking in eukaryotes. C. eleganis may therefore serve as a unique animal model of an obligate heme auxotroph to genetically and cell biologically delineate the pathways for heme homeostasis that has heretofore been elusive. Identification of molecules involved in heme homeostasis should permit selective drug-targeting of helminthic heme transport and heme-dependent cellular pathways that discriminate the parasite from its host, and significantly reduce chronic morbidity and debilitation in affected individuals.

For purposes of clarity, the figures referred to in this specification are described below in detail.

FIG. 1A depicts a systematic model of heme homeostasis in eukaryotic cells with currently unknown heme pathways marked with a “?”.

FIG. 1B depicts heme transport through the apical intestinal surface in the nematode C. elegans

FIG. 2A depicts the ultrastructure of C. elegans polarized intestinal cell in an electron micrograph cross-section of a pair of worm intestinal cells.

FIG. 2B depicts a close-up of C. elegans microvilli on the apical surface of the intestinal cell shown in FIG. 2A.

FIG. 3A is a reduced-minus-oxidized cytochrome absorption difference spectra of total extracts obtained from C. elegans wild-type strain N2 grown in defined CeHR media (containing 19 μM heme).

FIG. 3B is a Reduced-minus oxidized absorption spectra of pyridine hemochromes from either C. elegans mitochondrial and cytosolic fractions (scans 1 and 2), or from total extracts obtained from heme defective mutants of yeast (S. cerevisiae) and E. Coli (scans 3 and 4).

FIG. 4A shows the need of C. elegans for heme for growth and reproduction using synchronized L1 larvae as the primary noculum to analyze for aerobic growth in CeHR defined media.

FIG. 4B depicts a quantitative assessment of C. elegans growth in the presence of increasing amounts of hemin chloride.

FIG. 5A is an absorption spectra of pyridine Lemochrome obtained from synthesized heme using protoporphyrin IX and ferrous chloride as substrates.

FIG. 5B is a Reduced-minus-oxidized absorption spectra of pyridine hemochranes extracted from intact N2 C. elegans with methylethyl ketone. Commercial hemin was used as a standard.

FIG. 5C depicts fluoresence determined in live worms by excitation of porphyrin in the FITC channel using a Leica Fluorescent Microscope fitted with CCD Digital Imaging (40×).

FIG. 6 depicts an overall scheme of a forward genetic screen.

FIG. 7 pertains to heme auxotrophy of worms.

Figure (A) depicts a dithionite-reduced minus ferricyanide-oxidized absorption spectra of pyridine hemochromes from total homogenate, membrane- and cytosolic-enriched fractions of C. elegans grown in axenic mCeHR medium supplemented with 20 μM hemin chloride. A peak at 557 nm and trough at 541 nm indicates pyridine protohemochrome. All samples were reduced with 5 mM sodium dithionite or oxidized with 1 mM potassium ferricyanide. The vertical bar represents a ΔA of 0.005 for total homogenate, 0.0 12 for membrane fraction and 0.02 for cytosolic fraction. Inset: Immunoblot of the same samples (50 μg) that were separated by 4-20% SDS/PAGE and probed with ATP2p antisera followed by chemiluminescent detection. This immunoblot was stripped to remove ATP2p antibodies and re-probed with alpha-tubulin antibody.

Figure (B) is an ultra low-temperature spectrum of whole homogenate from C. elegans grown in mCeHR medium supplemented with 20 μM hemin. Only alpha bands are indicated for cytochrome c, b and oxidase (a+a3). The vertical bar represents a ΔA of 1.0.

Figure (C) shows aerobic growth of C. elegans in mCeHR medium supplemented with 0.20 μM hemin chloride, or 20 μM protoporphyrin IX (disodium salt). Equal numbers of synchronized L1 larvae were used as primary inoculum in 24-well plates in triplicate and the cultures analyzed quantitatively for growth at days 1, 3 and 7.

Figure (D) is a biphasic response of C. elegans cultured in the presence of increasing amounts of hemin chloride (μM). Equal numbers of synchronized L1 larvae were grown in 24-well plates in mCeHR medium for 9 days and quantified (worms/μl) by microscopy. Each data point represents the mean±SD from three separate experiments performed in triplicate.

Figure (E) depicts metabolic labeling in C. elegans cultured in the presence of heme. Synchronized L1 larvae were grown in mCeHR medium containing either ⁵⁹Fe or [⁵⁹FE]heme (9.4×10⁶ DPM) and the worms harvested as gravid adults. Heme was extracted and concentrated, and then resolved by TLC followed by detection with a PhosphorImager (top panel). Lane 5, [⁵⁹Fe]heme control. Radiolabeled bands were quantified in a gamma counter and CPM normalized to total protein (bottom panel). To correct for non-specific binding of the radiolabeled Fe and heme, parallel experiments were conducted in the presence of 1 mM sodium azide (samples 1 and 3).

FIG. 8 pertains to the characterization of heme uptake in C. elegans.

FIG. 8(A) depicts aerobic growth of C. elegans in mCeFIR medium with 20 μM hemin supplemented with either gallium protoporphyrin IX (GaPP) or gallium salts. Synchronized L1 larvae were grown for 9 days in 24-well plates and quantified (worms/μl) by microscopy. Each data point represents the mean from a single experiment, and each experiment was performed in triplicate. Inset depicts the GaPP analysis at lower concentrations for clarity.

FIG. 8(B) shows the effect of heme on the cytotoxicity of GaPP. Synchronized L1 larvae were inoculated in 24-well plates containing mCeHR medium with either 0, 2, 4, or 6 μM GaPP and increasing hemin (μM). The number of worms per μl was measured on day 9 and the data are presented as mean±SD.

FIG. 8(C) shows fluorescent metabolic labeling of worms with either 40 μM hemin (images 1, 4) or 40 μM ZnMP/4 μM hemin (images 2, 3, 5, 6) for 3 h followed by confocal microscopy with a 546 laser (images 1-3) and DIC optics (images 46). Arrowheads indicate ZnMP fluorescence accumulation within intestinal cells and developing embryos. For clarity, the boxed image in 2 is magnified in images 3 and 6. (Bar=100 μm).

FIG. 8(D) shows worms were incubated with 40 μM ZnMP/4 μM hemin for 16 h followed by a chase with 40 μM hemin. Worms were analyzed by epifluorescence microscopy (TRITC channel) and DIC optics. Experiments were performed either in the absence (images 1-4) or presence (images 5-8) of NaN₃ during the chase periods to test for the non-specific loss of ZnMP fluorescence. Photomicrograph 4 is shown at a lower power to depict the complete loss of ZnMP fluorescence. (Bar=100 μm). For (C) and (D), four separate experiments were performed with a minimum of 50 worms per data point per experiment. The data are representative for >90% of worms analyzed.

FIG. 9 shows that worms utilize heme-iron under iron deprivation. Equal numbers of synchronized L1 larvae were grown in the presence of 4, 20, and 100 μM hemin, either in basal mCeHR medium (set 1), or basal medium lacking exogenous iron (set 2), or as set 2 with 1 μM of the iron chelator ferrozine (set 3), or as set 3 with 486 μM ferrous ammonium sulfate (set 4). These values of ferrozine and iron were empirically determined by performing dose-response experiments and analyzing worm growth. The number of worms per μl was measured on day 9 and the data presented as mean±SD performed in triplicate. P<0.001 between sets 1 and 3. Within each set, values with different letters are significantly different. * denotes significant differences with the corresponding heme concentrations in set 1.

FIG. 10 is a phylogenetic maximum parsimony tree which shows that the losss of the heme pathway is commong among free-living and parasitic nematodes.

FIG. 11 illustrates the overall scheme of Gene Chip analysis.

FIG. 12 illustrates an example of snip-SN(RFLP) mapping using bulked segregant analysis.

FIGS. 13 (A), (B), (C) and (D) illustrate heme-dependent growth phenotype of ten heme-resistant mutants.

Supplementary Methods and Experimental Details

Biological Materials and Strains. Worm strains used were C. elegans wild-type N2 strain, Panagrellus redivivus LKC1 0 and Oscheius myriophila DFSO2O. E. coli strains were wild-type DH10B, RP523 (ALAD mutant), and Delta-vis (ferrochelatase mutant). S. cerevisiae haploid strains wild-type BY4743 and ferrochelatase knock-out mutant HEM15 were purchased as diploids from Open Biosystems, Huntsville, Ala. In the HEM15 mutant, the YOR176W open-reading frame corresponding to the HEM15 gene was replaced with a KanMX cassette.

Enzyme assays and Heme Determinations. Free-living worms (˜10⁶) cultured in mCeHR medium were washed three times in cold M9 buffer before resuspension in ice-cold 0.1 M Tris-HCl buffer, pH 8.0 containing a protease inhibitor cocktail (Calbiochem). The worm suspensions were homogenized by passage through a French Pressure Cell at an internal pressure up to 18,000 psi till breakdown of the worm cuticle occurred (>90%) as monitored by microscopy. The homogenate was clarified at 3000×g to remove cell debris, and the supernatant thus obtained was further centrifuged at 7000×g to obtain a mitochondrial-enriched pellet. This procedure provided >70% enrichment of mitochondrial membrane proteins as determined by immunoblots using ATP2 antisera. Parasitic worms were homogenized by grinding to a fine powder with a mortar and pestle in the presence of liquid N₂ and the homogenates were subsequently processed as above to obtain cytosolic and mitochondrial enriched fractions. Activities for ALAD, PBGD, succinate dehydrogenase and ferrochelatase enzymes were determined as described previously. All samples were analyzed using a Shimadzu dual beam scanning spectrophotometer UV-1601. Data are expressed as the average of triplicates, and enzyme activities normalized to total protein concentration, as determined by the Bradford assay (BioRad). Total hemes were analyzed by recording pyridine hemochrome spectra in aqueous alkaline pyridine solutions after reduction with 5 mM sodium dithionite and oxidation with 1 mM potassium ferricyanide, as described. Low temperature spectra (−191° C.) of cell extracts were obtained as described previously with an optical path length of 1 mm with one sheet of wet filter paper in the reference path.

Immunoblots and Worm Fractionations. All procedures were performed at 4° C. Worms were washed extensively in M9 buffer and suspended in MESH (220 mM mannitol, 2 mM EDTA, 70 mM sucrose, 5 mM HEPES, pH 7.4) with a protease inhibitor cocktail (Calbiochem Corp.). The worm suspensions were disrupted once by passage through a French Pressure Cell (<6000 psi), followed by homogenization with 10 strokes of a dounce homogenizer. The homogenates were centrifuged twice at 1000×g for 10 min. to remove cuticle and large debris, followed by centrifugation at 100,000×g to obtain pellets enriched in organelles and membranes, and supernatant fractions, enriched for cytosol. The pellet was resuspended in MESH and protein determined by the Bradford assay (BioRad). For immunoblotting, lysates were heated at 100° C. for 10 min. in the presence of SDS sample buffer containing (β-mercaptoethanol and centrifuged for 5 min. at 16,000×g at 4° C. Proteins were separated by SDS-PAGE, transferred to nitrocellulose, and detected by either the SuperSignal West Pico or West Femto Chemiluminescence kits (Pierce) using goat anti-rabbit and anti-mouse horseradish peroxidase conjugated secondary antibody (Pierce). Primary antibodies used in this study were rabbit polyclonal antibody against ATP2p (1:2000) and mouse monoclonal antibodies DM-1A to α-tubulin (Sigma, 1:500).

Determination of 18 S rDNA Gene Sequence and Phylogenetic Sequence Analysis. Genomic DNA from Trichuris suis larvae were isolated by standard procedures involving Proteinase K treatment and phenol-chloroform extractions. The 1 8S SSU rDNA gene from T. suis was amplified by PCR using genomic DNA as template and redundant primer mixes kindly provided by W. K. Thomas, University of New Hampshire (http://nematol.unh.edu/). The PCR product thus obtained was purified, sequenced and the DNA sequence was deposited in GenBank under accession number AY856093. Sequences for the same segment of the small subunit (SSU) of the 18S rDNA were collected to illustrate taxa tested in this study with appropriate phylogenetic resolution needed, as demonstrated for some other helminths. The closest taxa to two that were unavailable in GenBank, Haemonchus contortus and Ostertagia ostertagi, were selected based on taxonomy and a BLAST search (Dec. 15, 2004) of the closest available large subunit 28S sequences where taxon representation was denser in the database. For H. contortus, another strongylid, Ostertagia ostertagi AF036598 was used. For Ancylostoma caninum, another hookworm, Necator americanus AY295811, was used. To illustrate the phylogenetic position of the studied taxa among related eukaryotes, other slowly-evolving non-studied taxa were selected. The tree was rooted with chordates, Xenopus laevis (Craniata; Vertebrata) and Branchiostoma floridae (Cephalochordata). Other taxa included a priapulid worm, Priapulus caudatus (Priapulida), the horsehair worm Gordius aquaticus (Nematomorpha; Gordioida), arthropods Scolopendra cingulata (Myriapoda), Panulirus argus (Crustacea), and Tenebrio molitor (Hexapoda, Insecta) and flatworms Monocelis lineata (Turbellaria) and Echinobothrium chisholmae (Cestoda). The beginning nucleotide of the sequences for all taxa corresponds to position 984 of the C. elegans rDNA gene. An alignment was made using ClustalW (v1.8), manually checked for the presence of conserved positions among sequences and trimmed in GeneDoc. Phylogenetic analysis was made on a Clustal W multiple sequence nucleotide alignment (2082 character) using default parameters. This was run in PAUP*, ver.

4.0b4a where all characters were weighted by the maximum RC index value and characters sampled with equal probability. A Maximum Parsimony heuristic search employing TBR (tree bisection-reconnection) branch-swapping and ACCTRAN (accelerated transformation) character-state optimization, was bootstrapped 1,000 times.

FIG. 10, as noted above, provides a phylogenetic tree showing relative positions of the taxa used in this study. Tree based on a Clustal W multiple nucleotide sequence alignment (2082 character) of the SSU I 8S rDNA for parasitic and free-living roundworms (Nematoda), flatworms (Platyhelminthes) and horsehair worms (Nematomorpha). Their phylogenetic positions are shown relative to free-living chordates, insects and a priapulid worm. F=Free-living, P=Parasitic, (P)=parasitic as juveniles. Taxa in bold were assayed, or substituted for one assayed, for heme biosynthesis enzymes, in this study. Nineteen species included designated outgroups Xenopus laevis X04025 and Branchiostoma floridae M97571. Other taxa included Priapulus caudatus X87984; arthropods Scolopendra cingulata U29493, Panulirus argus AY743955, and Tenebrio molitor X07801; flatworms Monocelis lineata U45961, Echinobothrium chisholmae AF286986, and Schistosoma mansoni U65657; horsehair worms Paragordius varius AF42 1772 and Gordius aquaticus X87985; and nematodes worms Paragordius varius AF42 1772 and Gordius aquaticus X87985; and nematodes Trichuris suis AY856093, Ascaris suum U94367, Panagrellus redivivus AF083007, Strongyloides stercoralis AF279916, Caenorhabditis elegans X03680, Oscheius myriophila AF082994, Necator americanus AY29581 1 substituted for Ancylostoma caninum, Ostertagia ostertagi AF036598² substituted for Haemonchus contortus. Alignment having 18% parsimony-informative characters weighted by the maximum Resealed Consistency (RC) index value where characters were sampled with equal probability, and 67.5% characters were weighted as 1. Maximum parsimony phylogram pictured from a heuristic search employing TBR (tree bisection-reconnection) branch-swapping and ACCTRAN (accelerated transformation) character-state optimization, bootstrapped 1,000 times as implemented in PAUP*, ver. 4.0b4a. Tree Length=1204 Consistency Index=0.807, Retention Index=0.770, Rescaled Consistency Index=0.621, Homoplasy Index=0.193, Goloboff-Fit=−224.894.

Catalogues of Worms or Nematodes. In accordance with another aspect of the present invention, a catalogue of nematodes with various mutants and alleles is provided. For example, the nematode may be C. elegans, in which case the catalogue contains C. elegans and various mutants and alleles.

However, the catalog may be based upon any infections parasitic nematode, such as Ascaris suum, Trichuvis suis, Haemonchus contortus, Strongytoides stercoralis, Ancyclostoma duodenale and/or Ancyclostoma caninum, for example.

In each case, the catalogue contains a sample of each one of the above nematode with sample of corresponding mutants and alleles thereof.

However, it is preferred that the catalogue contain C. elegans and samples of mutants and alleles thereof. Such a catalogue may be used advantageously in modelling and studying mammalian heme transport mechanisms.

As used herein, the term “mutant” means a worm or nematode having one or more structural gene deletions or additions relative to the predominant background or control genome. The term “allele” means an alternative form of a gene or genes found at the same locus on homologous chromosomes.

Recently, Caenorhabditis elegans became the first animal and more importantly, the first multicellular organism, to have the sequencing of its genome essentially completed (C. elegans Consortium, Science 282:2011-2045, 1998). This is a landmark accomplishment for all of biology since we can now begin to investigate the phenomena that made cells come together and function in a complex multicellular system. The genetic blueprint (DNA) of C. elegans consists of ˜97 million base pairs mapped onto six pairs of chromosomes and including some 20,778 genes encoding proteins contained in a mere 959 cells (among which are 302 neurons). This provides biologists for the first time with a view of all the genes present in an animal. The only previous eukaryote with a sequenced genome is the yeast Saccharomyces cerevisiae, which is unicellular. Proteins unique to the nematode (and not yeast) may well define metazoans. Other comparisons of bacterial, yeast, nematode, plant, mouse and human genomes will reveal unique and surprising aspects of the genetic make-up of organisms.

The transparent body of C. elegans, its near-microscopic size (<1 mm), ease of culture and rapid life cycle simplified questions raised in the study of systems in humans, mice and even fruit flies. The nematode produces adult hermaphrodites that allow both outcrossing and selfing for genetic analyses. The developmental fate and connections of each of the nearly 1,000 cells in the adult nematode are known.

The availability of the C elegans genome sequence facilitates isolation of genes of interest in plant-parasitic helminths by using genes cloned from C. elegans, for example, as probes. The isolation of genes controlling nematode surface identity is one example demonstrating the utility of C. elegans genetic information. Collagens and cuticulins are important structural proteins in nematode cuticles. During molting and development, the cuticle of plant-parasitic nematodes undergoes biochemical changes. A probe made from a C. elegans cuticulin gene (Cut-1) was used to screen a genomic library of the parasitic root-knot nematode Meloidogyne artiellia. Sequence analysis revealed very similar promoter regions, and 75% homology at the amino acid level. The promoter regions of collagen genes (Col-2 and Col-6) were also highly homologous between C. elegans and M. artiellia. For less conserved gene sequences, PCR-based approaches can be designed using degenerate primers. Primers may also be designed on the basis of partial amino acid sequences of a gene product. The resultant PCR product can be used as a probe to isolate the gene of interest.

Transformation

DNA transformation may be effected, and has been effected, (involving microinjection of DNA into adult gonads) for C. elegans. Several animal-parasitic genes have been introduced and expressed in C. elegans.

Distinctions of the Genetics of C. elegans

About 58% of the putative coding regions of the C. elegans genome appear to be nematode-specific. This represents ˜400 distinct protein domains (Blaxter, 1998). Nematodes differ from other organisms in the following distinct ways (Blaxter, 1998):
(i) About 80% of C. elegans genes are trans-spliced to a common spliced leader exon.
(ii) About 20% of C. elegans genes are organized as operons (i.e., cotranscribed sets of two or genes).
(iii) Nematodes have a functional glyoxylate cycle (that enables formation of carbohydrates from fatty acids) and can synthesize polyunsaturated fatty acids de novo.
(iv) Differences exist in the biosynthesis of the cuticle, for example the existence of SXC (six-cysteine) domains in the surface coat of animal-parasitic nematodes. The SXC motif is most likely involved in protein-protein interactions.
(v) Nematodes possess surface-located lipid-binding proteins (thought to play roles in nutrient scavenging from the host or transport of lipid within animal-parasitic nematodes). Examples include the Nematode polyprotein allergen (NPA) and the Lipid-binding protein (LBP-20) which also has homologs in the plant-parasitic nematode Globodera pallida.

Amplifications using primers designed from the genome sequence of interest are used to facilitate molecular cloning of genetic regions of interest. Transformation of particular genetic regions into wild type will reveal any enhancement or suppression of phenotypes. Fusion of DNA sequences (with or without promoter regions) to the green fluorescent protein (GFP) reporter gene has facilitated studies of spatial and temporal expression profiles of C. elegans genes and screening of mutants. Two powerful technologies that can prove the necessity of a gene or its orthology include (i) deliberate construction of hybrid genes to cause misexpression based on deletions in specific genes, and (ii) RNA interference (RNAi) wherein candidate genes are inactivated by injection of double stranded RNA.

Among the some 20,778 structural genes in the genome of C. elegans, some 308 are implicated in heme homeostatis. These genes are and listed and described in some of the Tables below. Table V(A) shows eight (8) categories of C. elegans genes are characterized by the various column headings. All of the genes in the table have a fold change >1.6. Redundant genes have been removed.

Table V(B) again shows eight (8) categories of C. elegans genes as characterized by the same column headings. All collagen genes (27 genes) were removed from the list.

Table V(C) again shows (8) categories of C. elegans genes as characterized by the same column headings. Genes repeated more than 4 times were removed from the list.

Table VI shows a listing of heme resistant mutants of C. elegans characterized to date.

TABLE V

| | hemin | | Total | Putative Domains/Motifs | | | | orthologies | | known genes |
|---|---|---|---|---|---|---|---|---|---|---|
| Category | 4 μM | 600 μM | Genes | TMDs | Transporters | MeCo | Nucleic Acid | worms | Humans | in humans |

A. All the genes with the fold change >1.6 (Redundant genes have been removed).

| | | | | | | | | | | |
|---|---|---|---|---|---|---|---|---|---|---|
| 1 | ↑ | ↑ | 27 | 9 | 3 | 2 | 1 | 2 | 13 | 4 |
| 2 | ↓ | ↓ | 32 | 17 | 2 | 0 | 2 | 2 | 16 | 7 |
| 3 | ↓ | ↑ | 11 | 1 | 0 | 1 | 0 | 0 | 5 | 1 |
| 4 | ↑ | ↓ | 14 | 6 | 4 | 1 | 0 | 3 | 13 | 7 |
| 5 | [symbol] | ↑ | 86 | 23 | 6 | 8 | 2 | 7 | 50 | 16 |
| 6 | [symbol] | ↓ | 54 | 19 | 1 | 3 | 2 | 4 | 40 | 18 |
| 7 | ↑ | [symbol] | 58 | 16 | 2 | 5 | 4 | 6 | 27 | 10 |
| 8 | ↓ | [symbol] | 27 | 8 | 3 | 1 | 1 | 4 | 16 | 7 |
| Total | | | 307 | 99 | 21 | 21 | 12 | 28 | 180 | 70 |

B. All collagen genes according to wormbase were removed from the list. (27 genes)

| | | | | | | | | | | |
|---|---|---|---|---|---|---|---|---|---|---|
| 1 | ↑ | ↑ | 24 | 6 | 3 | 2 | 0 | 2 | 10 | 2 |
| 2 | ↓ | ↓ | 28 | 18 | 2 | 0 | 1 | 1 | 12 | 4 |
| 3 | ↓ | ↑ | 11 | 1 | 0 | 1 | 0 | 0 | 8 | 1 |
| 4 | ↑ | ↓ | 10 | 8 | 4 | 1 | 0 | 3 | 9 | 5 |
| 5 | [symbol] | ↑ | 65 | 22 | 6 | 7 | 1 | 7 | 49 | 15 |
| 6 | [symbol] | ↓ | 47 | 13 | 1 | 3 | 0 | 3 | 33 | 11 |
| 7 | ↑ | [symbol] | 49 | 10 | 2 | 5 | 4 | 3 | 20 | 5 |
| 8 | ↓ | [symbol] | 26 | 8 | 3 | 1 | 1 | 4 | 15 | 6 |
| Total | | | 280 | 76 | 21 | 20 | 7 | 23 | 153 | 49 |

C. Genes repeated more than 4 times were removed from the list.

| | | | | | | | | | | |
|---|---|---|---|---|---|---|---|---|---|---|
| 1 | ↑ | ↑ | 17 | 4 | 1 | 1 | 0 | 2 | 5 | 1 |
| 2 | ↓ | ↓ | 26 | 12 | 2 | 0 | 1 | 1 | 10 | 4 |
| 3 | ↓ | ↑ | 7 | 1 | 0 | 0 | 0 | 0 | 4 | 1 |
| 4 | ↑ | ↓ | 5 | 2 | 2 | 1 | 0 | 0 | 4 | 3 |
| 5 | [symbol] | ↑ | 64 | 19 | 6 | 7 | 1 | 7 | 38 | 10 |
| 6 | [symbol] | ↓ | 44 | 13 | 0 | 2 | 0 | 2 | 30 | 10 |
| 7 | ↑ | [symbol] | 47 | 9 | 2 | 5 | 4 | 2 | 18 | 4 |
| 8 | ↓ | [symbol] | 24 | 8 | 3 | 0 | 1 | 4 | 13 | 4 |
| Total | | | 234 | 88 | 16 | 16 | 7 | 18 | 122 | 37 |

↑: up registered,
↓: down registered, [symbol]
: no change
TMDs: transmembrane domains,
Transporters: Permeases, ABC, ATPase, etc,
MeCo: Meta [symbol] cofactor binding sites including heme
Nucleic Acid: DNA and RNA binding
Known in worms: Worm genes with mutants or ascribed functions in WormBase
Orthologe: Human orthologe is assigned when E value is [symbol] than 10e−4
Known in human: Human orthologies with mutants or ascribed functions according to publications in NCBI.
*genes for 59 collagen and XXX GST were subtracted from 306 genes to get this number
Note:
Genes removed include: 27 collagens, 15 genes containing C-type lactin domains, 13 genes containing [symbol] TK domains, 7 GSTs, 7 genes containing UDP- [symbol] transferase domains and 4 vit.

TABLE VI
of heme resistant mutants charcterised to date

| | | | | | | | | | | Growth* | | |
|---|---|---|---|---|---|---|---|---|---|---|---|---|
| | Selection | morpho- | Generation time (Days) | | | brood size* | | | % | 1.6 μM | 800 μM | 0.5 μM |
| Mutants^{†} | hemin (μM) | logy | 1.5 μM | 20 μM | 800 μM | 1.5 μM | 20 μM | 800 μM | growth^{‡} | hemin | hemin | GaPP |
| IH728 | 800 | wild type | | 3.5 | 4 | | 53 | | 25.6 | ++++ | ++++ | ++++ |
| IH938 | 800 | wild type | | 3.5 | 4 | | 72 | | 27.1 | ++++ | ++++ | ++++ |
| IH731 | 1000 | wild type | | 3.5 | 4 | | 53 | | (—) | ++++ | ++++ | ++++ |
| IH828 | 800 | wild type | | 3.5 | 4 | | 67 | | 34.6 | ++ | ++ | ++++ |
| IH1058 | 800 | wild type | | | | | | | (—) | ++ | ++ | ++++ |
| IH921 | 1000 | wild type | | | | | | | 23.2 | ++ | ++ | ++++ |
| IH1068 n | 800 | wild type | | 4 | | | | | (—) | + | ++ | + |
| IH1068 d | 800 | dumpy | | | 4 | | 12.4 | | 22.6 | + | ++ | + |
| IH1048 | 800 | wild type | | | 4 | | | | 26.1 | + | ++ | + |
| IH718 | 800 | wild type | | | | | | | 36.8 | + | ++ | + |
| Control | n/a | | | 3.5 | >16 | | 62.3 | | | + | ND | + |
| Wild-type (N2) | | | | | | | | | | | | |

+ = wild type equivalent In growth
ND: No growth,
n/a: not applicable,
(—) not analyzed currently
*average brood size in 24 well plates in triplicates
^{†}Mutants characterized to-date. Two additional mutants (long) are being currently backcrossed.
^{‡}heme resistance worms from heterozygous parents to show recessive allales

For Table V All genes with fold change >1.6/.

| Affy ID | Wormbase | Wormbase | | humans | Knownhuman |
|---|---|---|---|---|---|
| >2(all inc) | | | | | |
| 176851_at | Y40B10A | Y40B10A.6 | O-methyl transferase domain | + | — |
| 180150_at | F54E2 | F54E2.1 | Unknown | − | — |
| 184035_at | M02F4 | M02F4.7 | C-type lectin domain | + | — |
| 189419_at | F15B9 | F15B9.6 | Phospholipase domain | − | — |
| 176681_at | F35B3 | F35B3.4 | Fibronectin domain | − | — |
| 179098_at | T19C9 | T19C9.8 | Unknown | − | — |
| 183117_at | ZK742 | ZK742.3 | NADH: Flavin oxireductase domain | − | — |
| 183379_at | K01D12 | K01D12.9 | Unknown | − | — |
| 183676_s_at | F08F8 | F08F8.5 | Unknown | + | — |
| 185399_at | Y75B8A | Y75B8A.28 | Unknown | − | — |
| 186383_at | T19D12 | T19D12.4a, T19D12.4b, T19D12.4c, | Von Willebrand factor A domain | + | COI12A1, TTBK2 |
| | | T19D12.5 | | | |
| 187962_at | K02E2 | K02E2.4 | ins-35 | − | — |
| 190134_s_at | T10B10 | T10B10.1 | COL-41 | + | COL5A1 |
| 190693_at | K08B4 | K08B4.3 | UDP-glucuronosyl and UDP-glucosyl | + | — |
| | | | transferase domain | | |
| 191418_at | ZC443 | ZC443.6 | UDP-glucuronosyl and UDP-glucosyl | + | — |
| | | | transferase domain | | |
| 180973_at | F49F1 | F49F1.6 | ShTK domain | + | mucin-2 |
| | | 16 | | 8 | 3 |
| 1.6-2(all inc) | | | | | |
| 173836_at | T24A11 | T24A11.3 | Toh-1 | + | — |
| 177024_at | C29E4 | C29E4.1 | COL-90 | + | — |
| 182573_at | T05B4 | T05B4.3 | ShTK domain | − | — |
| 184313_s_at | K11H12 | K11H12.4 | Unknown | − | — |
| 184399_at | Y26D4A | Y26D4A.10, Y26D4A.11 | Unknown | − | — |
| 185343_at | T28C12 | T28C12.6 | Unknown | − | — |
| 185425_at | Y38E10A | Y38E10A.5 | C-type lectin domain | + | — |
| 187662_at | F32H5 | F32H5.3a, F32H5.3b | Unknown | − | — |
| 188028_at | F07C3 | T18H9.1 | grd-6 | + | mucin-2 |
| 192059_at | Y51A2D | Y51A2D.4 | MFS domain | + | — |
| 194067_at | F35C5 | F35C5.9 | C-type lectin domain | − | — |
| | | 11 | | 5 | 1 |
| >2(All Dec) | | | | | |
| 175239_at | F15E11 | F15E11.15 | Unknown | − | — |
| 186519_at | F15E11 | F15E11.12, F15E11.15 | Unknown | − | — |
| 183666_at | C52D10 | C52D10.13 | COL-183 | + | COL3A1 |
| 183724_at | Y11D7A | Y11D7A.5 | Unknown | − | — |
| 188391_at | F59E12 | F59E12.12 | bli-2 | + | COL3A1 |
| 189482_s_at | C35B8 | C35B8.1 | COL175 | + | — |
| 171941_s_at | F44E5 | F44E5.5 | Hsp70 domain | + | — |
| 172400_x_at | F22A3 | F22A3.6a, F22A3.6b | Destabilase domain | − | — |
| 172971_s_at | Y54G2A | Y54G2A.11a, Y54G2A.11b | Myb domain | + | — |
| 176395_at | Y71G12B | Y71G12B.18 | Unknown | − | — |
| 177613_at | F57G8 | F57G8.7 | Unknown | − | — |
| 177812_at | F10C2 | F10C2.7 | MFS domain | + | NA/PI-4 |
| 178087_s_at | F58B3 | F58B3.2 | Lys-5 | − | — |
| 180616_at | T22B7 | T22B7.3 | Amidinotransferase domain | − | — |
| 180706_at | K06H6 | K06H6.2 | Unknown | − | — |
| 181502_s_at | W07A12 | W07A12.6 | Acyltransferase domain | − | — |
| 181520_at | W07A12 | W07A12.7 | Acyltransferase domain | − | — |
| 183273_at | C14C6 | C14C6.3 | Glycosyl transferase domain | − | — |
| 185242_at | Y105C5B | Y105C5B.7 | Calcineurin like phosphoesterase | + | — |
| | | | domain | | |
| 188495_at | F09G2 | F09G2.3 | Phosphate transporter domain | + | leukemia virus |
| | | | | | receptor 1 |
| 189595_s_at | K10C2 | K10C2.3 | Aspartyl protease domain | + | — |
| 190958_s_at | F44E5 | F44E5.4 | Hap70 domain | + | HSP70AB |
| 191882_at | F47C10 | F47C10.6 | UDP-glucuronosyl and UDP-glucosyl | + | — |
| | | | transferase domain | | |
| 193588_s_at | F28D1 | F28D1.5 | thn-2 | − | — |
| | | 24 | | 11 | 5 |
| 1.6-2(all dec) | | | | | |
| 180880_at | K06H6 | K06H6.1 | Unknown | − | — |
| 181819_s_at | Y37D8A | Y37D8A.4 | SH2 domain | + | — |
| 181902_at | Y43D4A | Y43D4A.5 | Unknown | + | MUC-1 |
| 187382_at | F55C10 | F55C10.4 | Unknown | − | — |
| 187846_at | W03F11 | W03F11.5a, W03F11.5b | Unknown | − | — |
| 189864_s_at | F19C7 | F19C7.7 | COL-110 | + | COL4A5 |
| 189975_at | F08H9 | F08H9.5 | C-type lectin domain | + | — |
| 193034_at | F56H6 | F56H6.5 | GDP-mannose 4,6-dehydratase domain | + | — |
| | | 8 | | 5 | 2 |
| >2 (De4/In500) | | | | | |
| 180562_at | B0218 | B0218.8 | C-type lectin domain | + | — |
| 171932_x_at | F09C8 | F09C8.1 | Phospholipase domain | + | — |
| 172184_x_at | Y46C8AL | Y46C8AL.2 | Unknown | + | — |
| 178297_at | T24B8 | T24B8.5 | ShTK domain | − | — |
| 178843_at | F08G5 | F08G5.6 | Unknown | − | — |
| 179424_at | F27C8 | F27C8.4 | spp-18 | − | — |
| 182970_at | K10D11 | F55G11.4 | Unknown | − | — |
| 183527_s_at | C14C6 | C14C6.5 | ShTK domain | − | — |
| 187964_at | F54F3 | F54F3.3 | Lipase domain | + | — |
| 188441_at | F21F8 | F21F8.4 | Aspartyl protease domain | + | gastric cathepsin E |
| 192509_at | ZK666 | ZK666.6 | C-type lectin domain | − | — |
| | | 11 | | 5 | 1 |
| >2 (In 4/De500) | | | | | |
| 172134_x_at | F56B6 | F59D8.2 | Vit-4 | + | Apollipoprotein B- |
| | | | | | 100 |
| 173411_s_at | K07H8 | K07H8.6a, K07H8.6b, K07H8.6c | Vit-6 | + | — |
| 175993_at | C29E4 | C29E4.7 | GST domain | + | — |
| 184144_at | C05D9 | R193.2 | Von Willebrand factor A domain | + | COL6A3 |
| 188335_at | T05A1 | T05A1.2 | COL-122 | + | COL4A5 |
| 188947_at | T09F5 | T09F5.9 | C-type lectin domain | + | — |
| 189227_at | W07B8 | W07B8.1 | Cystein protease domain | + | Cathepsin B1 |
| 189660_at | F59D6 | F59D6.3 | Aspartyl protease domain | + | — |
| 189911_s_at | F26F12 | F26F12.1 | COL-140 | + | — |
| 190619_at | C15C8 | C15C8.3 | Aspartyl protease domain | + | gastric cathepsin E |
| 180752_s_at | D1054 | D1054.10 | Unknown | − | |
| 194239_x_at | F59D8 | F59D8.1 | Vit-3 | + | Apolipoprotein B- |
| | | | | | 100 |
| | | 12 | | 11 | 6 |
| 1.6 to 2 | | | | | |
| 177428_at | F58G6 | F58G6.3, F58G6.7 | Ctr domain | + | human Ctr1 |
| 188245_at | F15A2 | F15A2.1 | COL-184 | + | — |
| | | 2 | | 2 | 1 |
| >2(NC4/In500) | | | | | |
| 177816_at | F49H6 | F49H6.3 | Unknown | − | — |
| 178017_s_at | F01D5 | F01D5.1 | ShTK domain | + | — |
| 179933_at | F39E9 | F39E9.1 | Unknown | − | — |
| 180315_at | F44G3 | F44G3.10 | Unknown | − | — |
| 180727_at | F49F1 | F49F1.5 | ShTK domain | − | — |
| 181099_at | F54C1 | F54C1.1 | UDP-glucuronosyl and UDP-glucosyl | + | — |
| | | | transferase domain | | |
| 181946_at | C03H5 | C03H5.1 | C-type lectin domain | + | MMR |
| 183020_at | F48G7 | F48G7.8 | ShTK domain | − | — |
| 183526_at | C14C6 | C14C6.5 | ShTK domain | − | — |
| 183665_s_at | F48G7 | F48G7.5 | ShTK domain | − | — |
| 183850_at | Y48E1B | Y48E1B.8 | Unknown | − | — |
| 184116_s_at | T05E12 | T05E12.6 | Unknown | − | — |
| 184352_at | C17H12 | C17H12.6 | Unknown | − | — |
| | | C25H3.10, C25H3.10a, C2 | | | |
| 184624_s_at | C25H3 | 5H3.10b | F-box domain | − | — |
| 184707_s_at | C32H11 | C32H11.10 | dod-21 | − | — |
| 185145_at | Y46D2A | Y46D2A.2 | Unknown | − | — |
| 188106_at | T01C3 | T01C3.4 | Lipase domain | − | — |
| 188465_s_at | R09D1 | R09D1.8 | glycosyl hydrolases domain | + | — |
| 188987_at | F20G2 | F20G2.1 | Short chain dehydrogenese domain | + | — |
| 189971_at | F01G10 | F01G10.3 | ech-9 | + | PBFE |
| 190139_s_at | E03H4 | E03H4.10 | CUB domain | + | mannose receptor, |
| | | | | | C type 2 |
| 190830_at | T21C9 | T21C9.8 | Transthyretin like domain | − | — |
| 191502_at | ZC443 | ZC443.5 | UDP-glucuronosyl and UDP-glucosyl | + | — |
| | | | transferase domain | | |
| 191568_at | C13D9 | C13D9.9 | UDP-glucuronosyl and UDP-glucosyl | + | — |
| | | | transferase domain | | |
| 192076_at | K02G10 | K02G10.7a, K02G10.7b | aqp-8 | + | — |
| 193007_s_at | K08E7 | K08E7.9 | pgp-1 | + | P-glycoprotein |
| 194063_at | F35C5 | F35C5.7 | C-type lectin domain | − | — |
| | | 27 | | 11 | 4 |
| 1.5-2(NC4/In500) | | | | | |
| 172069_x_at | T10H4 | T10H4.12 | cpr-3 | + | Cathepsin B1 |
| 172503_x_at | W08E12 | W08E12.3 | 2Fe-2S-ferredoxin domain | + | — |
| 172769_x_at | F44C8 | F44C8.1 | cyp-33C4 | + | — |
| 173090_s_at | F35C5 | F35C5.8 | C-type lectin domain | + | C-type lectin |
| 175170_s_at | ZK6 | ZK6.10 | dod-19 | − | — |
| 175870_s_at | Y34F4 | Y34F4.4 | Unknown | − | — |
| 176007_at | Y50D4A | Y50D4A | Ribosome protein S2 domain | + | — |
| 176026_at | C06E1 | C06E1.3 | Unknown | + | — |
| 176360_at | R13A5 | R13A5.10 | Cytidine deaminase domain | + | — |
| 176756_s_at | Y65B4A | F56A6.1a, F56A6.1b | Piwi domain | + | — |
| 176826_at | Y40B10A | Y40B10A.2 | O-methyl transferase domain | + | — |
| 177059_at | Y46C8AL | Y46C8AL.5 | C-type lectin domain | − | — |
| 177187_at | Y46C8AR | Y46C8AR.1 | C-type lectin domain | − | — |
| 177219_at | M28 | M28.8 | Glutamine amidotransferase domain | − | — |
| 177693_at | R03G8 | R03G8.3 | Unknown | − | — |
| 177827_at | T19C4 | T19C4.5 | Unknown | − | — |
| 178128_at | F01D5 | F01D5.3 | ShTK domain | − | — |
| 178403_at | F10A3 | F10A3.2 | FTH domain | − | — |
| 179682_at | C31H5 | C31H5.6 | Acyl-CoA thioester hyrolase domain | + | — |
| 179989_at | F36G9 | F36G9.14 | FTH domain | − | — |
| 180379_s_at | C17F4 | C17F4.7 | Unknown | − | — |
| 180410_at | Y39G8B | Y39G8B.7 | ShTK domain | − | — |
| 180642_at | F53A9 | F53A9.2 | Peptidase M domain | + | — |
| 180810_at | R10D12 | R10D12.9 | MtN3/saliva family domain | + | — |
| 181459_at | Y102A5C | F49H6.13 | Unknown | − | — |
| 182106_at | C34H4 | C34H4.1 | Unknown | − | — |
| 182276_at | T10B10 | T10B10.4, T10B10.4a | Unknown | − | — |
| 182279_at | Y47H9C | Y47H9C.1 | Unknown | − | — |
| 182712_at | C49G7 | C49G7.4 | ShTk domain | − | — |
| 182790_at | F10G2 | F10G2.3 | C-type lectin domain | + | — |
| 183702_at | K05B2 | K05B2.4 | Acyl-CoA thioester hyrolase domain | + | — |
| 183867_at | C45E5 | C45E5.4 | Unknown | − | — |
| 186413_at | H20E11 | H20E11.1a, H20E11.1b | Unknown | − | — |
| 186528_s_at | F46E10 | F46E10.11 | Unknown | + | — |
| 186799_at | Y105C5B | Y105C5B.15 | Calcineurin like phosphoesterase | + | — |
| | | | domain | | |
| 186832_at | F27E5 | F27E5.1 | Acid ceramidase | + | Acid ceramidase |
| | | | | | precursor |
| 187548_s_at | C32D5 | C32D5.6 | Unknown | + | — |
| 188589_at | F33D11 | F33D11.3 | COL-54 | + | COL13A1 |
| 188648_at | C45B2 | C45B2.5 | Glutamine synthase domain | + | — |
| 189179_at | C30G7 | C30G7.1 | hil-1 | + | — |
| 189325_at | F02D8 | F02D8.4 | Zinc carboxylpeptidase domain | + | PCPB protein |
| 189473_at | Y39D8C | Y39D8C.1 | abt-4 | + | human ABCA3 |
| 190156_s_at | C48B4 | C48B4.1 | Acyl-CoA dehydrogenase domain | + | Acyl-coenzyme A |
| | | | | | isoform b |
| 190301_s_at | C05E11 | C05E11.5 | amt-4 | + | — |
| 190413_at | F25G6 | F25G6.6 | nrs-2 | + | Asparagine |
| | | | | | synthetase |
| 190899_at | F21H7 | F21H7.1 | GST-22 | + | Prostaglandin-D |
| | | | | | synthase |
| 190959_s_at | F37B1 | F37B1.5 | GST-16 | + | Prostaglandin-D |
| | | | | | synthase |
| 191298_s_at | F23B2 | F23B2.12 | pcp-2 | + | — |
| 191336_at | K06C4 | K06C4.8 | rhodopsin domain | + | — |
| 191406_at | F21D5 | F21D5.3 | Multicopper oxidase domain | − | — |
| 191541_at | C54D10 | C54D10.1 | GST domain | + | — |
| 191970_at | C34H3 | C34H3.2 | odd-2 | + | — |
| 192249_at | F22A3 | F22A3.1 | SAM/pointed domain | + | — |
| 192559_s_at | F35C5 | F35C5.8 | C-type lectin domain | + | CLECSF6 |
| 192596_s_at | T03F7 | T03F7.7a, T03F7.7b | CRAL/TRIO domain | + | SEC14-like 2 |
| 192610_at | C24F3 | C24F3.3 | nas-12 | + | — |
| 192628_at | F11A5 | F11A5.10 | glc-1 | + | — |
| 193165_at | F08F3 | F08F3.3 | rhr-1 | + | — |
| 193926_at | Y48A6B | Y48A6B.7 | Cytidine deaminase domain | + | — |
| | | 59 | | 39 | 12 |
| >2(NC4/De500) | | | | | |
| 176230_at | Y71H2AM | Y71H2AM.16 | Histidine acid phosphatase domain | + | Prostatic acid |
| | | | | | phosphatase |
| 177532_at | F22B5 | F22B5.4 | Unknown | − | — |
| 179226_at | C06B3 | C06B3.7 | Unknown | − | — |
| 180997_at | C34D4 | C34D4.3 | MSP domain | + | — |
| 183455_at | F26B1 | F26B1.1 | Unknown | + | — |
| 189366_at | K07C6 | K07C6.4 | cyp-35B1 | + | cytochrome P450, |
| | | | | | 2U |
| 190080_at | F26C11 | F26C11.1 | Histidine acid phosphatase domain | + | — |
| 190962_s_at | F37B1 | F37B1.8 | GST-19 | + | Prostaglandin-D |
| | | | | | synthase |
| 193584_s_at | ZK520 | ZK520.5 | cyn-2 | + | — |
| | | 9 | | 7 | 3 |
| 1.6-2(NC4/De500) | | | | | |
| 172904_x_at | K05F1 | K05F1.7 | msp-63 | + | — |
| 173632_s_at | M18 | M18.1 | COL-129 | + | COL4A5 |
| 173650_s_at | ZK265 | ZK265.2 | COL-63 | + | col1A1 |
| 174152_s_at | C09G5 | C09G5.6 | bli-1 | + | COL4A5 |
| 174273_at | F01G10 | F01G10.9 | Unknown | + | — |
| 174960_at | ZK1010 | ZK1010.7 | COL-97 | + | COL9A3 |
| 175898_at | ZK105 | ZK105.1 | Unknown | − | — |
| 176419_at | Y71G12B | Y71G12B.17 | Phosphatidylinositol transfer protein | + | phosphatidylinositol |
| | | | domain | | transfer protein |
| 176461_s_at | Y59H11AM | Y59H11AM.3 | Rhodanese-like domain | + | — |
| 177065_at | K09F5 | K09F5.2 | Vit-1 | + | — |
| 177729_at | E01G4 | E01G4.6 | Phospholipase domain | + | — |
| 178189_at | F28C6 | F28C6.5 | Unknown | − | — |
| 178243_s_at | F32B6 | F32B6.4 | Unknown | − | — |
| 178439_s_at | F17C8 | F17C8.7 | Unknown | − | — |
| 178759_at | F59C6 | F59C6.6 | nlp-4 | − | — |
| 178929_at | T03F7 | F47G9.3 | Zona pellucida-like domain | − | — |
| 179199_at | T09F5 | T09F5.1 | Galactosyltransferase domain | + | — |
| 180462_at | C07E3 | C07E3.10 | Unknown | − | — |
| 181331_s_at | K09C6 | K09C6.8 | Unknown | + | — |
| 181833_at | K01A2 | K01A2.4 | Unknown | + | — |
| 182078_at | T10E9 | T10E9.8 | Unknown | − | — |
| 182498_at | H25K10 | H25K10.1 | Calcineurin like phosphoesterase | + | — |
| | | | domain | | |
| 183232_s_at | K01A2 | K01A2.3 | Unknown | − | — |
| 183713_at | C24G7 | C24G7.2 | Amiloride-sensitive sodium channel | + | — |
| | | | domain | | |
| 184740_at | C33F10 | C33F10.1 | Unknown | − | — |
| 184839_at | T06C10 | C55F2.1b | ALCARFT/IMPCHase bienzyme domain | + | — |
| 184864_at | C08F11 | C08F11.11 | Unknown | − | — |
| 186349_at | Y51H7C | Y51H7C.1 | Collagen domain | + | mucin-2 |
| 186974_s_at | Y49F6B | Y49F6B.10 | COL-71 | + | COL3A1 |
| 187612_s_at | F47D12 | F47D12.7 | Kelch motif domain | + | — |
| 188128_at | C50B6 | C50B6.7 | Alpha-amylase domain | + | — |
| 188849_at | C56E6 | C56E6.2 | Ras domain | + | — |
| 188917_s_at | F26H11 | F26H11.2c, F26H11.2d | DDT domain | + | Fetal Alzheimer |
| | | | | | antigen isoForm 1 |
| 189208_at | F38B6 | F38B6.4 | Phosphoribosylglycinamide synthase | + | phosphoribosyl- |
| | | | domain | | glycinamide |
| | | | | | formyltransferase |
| 189762_at | K09H9 | K09H9.3 | COL-49 | + | COL5A1 |
| 189921_at | C05C10 | C05C10.4 | Histidine acid phosphatase domain | + | — |
| | | M02D8.4a, M02D8.4b, M02 | | | Asparagine |
| 190094_s_at | M02D8 | D8.4c | Asparagine synthase domain | + | synthetase |
| 190585_at | ZK970 | ZK970.7 | Unknown | − | — |
| 191303_at | Y53F4B | Y53F4B.32 | GST-29 | + | |
| 192175_at | C27D6 | C27D6.3 | Unknown | + | PARP1 protein |
| 192786_s_at | B0286 | B0286.3 | SAICAR synthase domain | + | — |
| 192819_at | K10C3 | Y67A6A.2 | nhr-62 | + | HNF4A |
| 193127_s_at | K07E3 | K07E3.3 | dao-3 | + | C-1- |
| | | | | | tetrahydrofolate |
| | | | | | synthase |
| 193885_at | B0393 | B0393.5 | EGF-like domain | + | Fibrillin 1 |
| 194086_at | W01F3 | W01F3.3 | Trypsin inhibitor domain | + | — |
| | | 45 | | 33 | 15 |
| >2(In4/NC500) | | | | | |
| 174713_at | F14F4 | F14F4 | mrp-5 | − | — |
| 175810_at | F52E1 | F52E1.1 | pos-1 | + | — |
| 177544_at | F58E6 | F58E6.7, F58E6.11 | Unknown | − | — |
| 179900_s_at | C44B7 | C44B7.5 | Cyt b-561/ferric reductase domain | − | — |
| 185275_at | C54D10 | K01D12.14 | cdr-5 | + | — |
| 186182_s_at | R02E12 | R02E12.6 | Unknown | + | — |
| 186232_s_at | C44B12 | C44B12.1 | Unknown | − | — |
| 189561_at | W03G11 | W03G11.1 | COL-181 | + | COL3A1 |
| 189671_at | C50H11 | C50H11.15 | cyp-33C9 | + | — |
| 192163_at | ZK1193 | ZK1193.1a | COL-19 | + | — |
| 173316_s_at | Y62H9A | Y62H9A.6 | Unknown | − | — |
| 173647_s_at | C53B4 | C53B4.5 | COL-119 | + | col1A1 |
| 174283_s_at | F38B6 | F38B6.4 | Phosphoribosylglycinamide synthase | + | — |
| | | | domain | | |
| 174964_at | K02B9 | K02B9 | Unknown | − | — |
| 175010_s_at | F57C2 | F57C2.4 | Unknown | − | — |
| 175377_at | ZK813 | ZK813 | Unknown | − | — |
| 176004_at | M02H5 | M02H5.4 | Zinc finger domain | + | HNF4A |
| 177253_at | R186 | R186.1 | Unknown | + | — |
| 177389_at | F58E6 | F58E6.8 | Unknown | − | — |
| 177820_at | ZC373 | ZC373.2 | Unknown | − | — |
| 178775_at | C01G6 | C01G6.3 | Unknown | − | — |
| 179525_at | F36H1 | F36H1.5 | Unknown | − | — |
| 179805_s_at | T04G9 | T04G9.7 | Unknown | − | — |
| 180173_at | Y62H9A | Y62H9A.4 | Unknown | − | — |
| 180306_at | C10G8 | C10G8.4 | Trypsin inhibitor domain | + | — |
| 180361_at | F42G2 | F42G2.2 | FTH domain | − | — |
| 180676_at | ZK813 | ZK813.1 | Chorion domain | + | — |
| 182492_at | C16C4 | C16C4.4 | MATH domain | − | — |
| 183721_at | F17E9 | F17E9.4 | Unknown | − | — |
| 185762_at | F47C10 | F47C10.2 | BTB domain | + | — |
| 186231_at | C44B12 | C44B12.1 | Unknown | − | |
| 188406_s_at | F11H8 | F11H8.3 | COL-8 | + | col3A1 |
| 188456_at | T15B7 | T15B7.3 | COL-143 | + | col7A1 |
| 190530_s_at | F11G11 | F11G11.11 | COL-20 | + | — |
| 192345_s_at | F32H5 | F32H5.1 | Cystein protease domain | + | Cathepsin B1 |
| | | 35 | | 17 | 6 |
| 1.6-2(In4/NC500) | | | | | |
| 172443_x_at | Y73F8A | Y73F8A.9 | pqn-91 | + | — |
| 175102_at | C04H5 | C04H5.7 | Unknown | − | — |
| 177489_at | C05E7 | C05E7.2 | Unknown | − | — |
| 178259_at | DY3 | F36A2.3 | Malate dehydrogenase domain | − | — |
| 180540_at | C42D4 | C42D4.3 | Fibronectin domain | − | — |
| 182415_s_at | F48E3 | F48E3.4 | Peptidase S1 and S6 domain | − | — |
| 182599_s_at | W02G9 | W02G9.4 | CUB domain | + | — |
| 182628_s_at | T21F4 | T21F4.1 | Arginase domain | + | Arginase 1 |
| 183115_at | C34H4 | C34H4.2 | Unknown | − | — |
| 183187_at | K10D11 | K10D11.1 | Unknown | − | — |
| 184393_at | C33H5 | C33H5.13 | Unknown | − | — |
| 184943_s_at | T02G5 | T02G5.11 | Nanos RNA binding domain | − | — |
| 185954_at | Y54G11A | Y54G11A.7 | Tetratricopeptide repeat domain | + | — |
| 185964_at | Y37D8A | Y37D8A.19 | Unknown | − | — |
| 187358_at | F07F6 | F07F6.5 | Unknown | − | — |
| 188747_at | Y54E10BL | Y54E10BL.2 | COL-48 | + | COL4A3 |
| 189178_at | F44G3 | F44G3.2 | ATP: guanidophosphotransferase | + | — |
| | | | domain | | |
| 189953_at | T11F9 | T11F9.3 | nas-20 | + | — |
| 191251_at | C33A12 | C33A12.6 | UDP-glucuronosyl and UDP-glucosyl | + | UDP- |
| | | | transferase domain | | glucuronosyl- |
| | | | | | transferase |
| | | | | | 2A1 |
| | | | | | precursor |
| 191470_s_at | C31C9 | C31C9.1a, C31C9.1b | tag-10 | + | mucin-2 |
| 192956_at | F11G11 | F11G11.3 | GST-6 | + | — |
| | | 21 | | 10 | 4 |
| >2(De4/NC500) | | | | | |
| 192194_s_at | W03G1 | W03G1.7 | asm-3 | + | SMPD1 |
| 174393_at | H40L08 | H40L08.2 | pseudogene predicted | − | — |
| 179360_s_at | T16G1 | T16G1.7 | Unknown | − | — |
| 179879_at | Y51H4A | Y51H4A.5 | Lipase domain | − | — |
| | | 4 | | 1 | 1 |
| 1.6-2(De4/NC500) | | | | | |
| 173051_s_at | F22A3 | F22A3.6a | Destabilase domain | − | — |
| 176474_at | Y54F10AM | Y54F10AM.6 | Unknown | − | — |
| 176864_at | Y5H2A | Y5H2A.1 | Unknown | − | — |
| 177375_at | M60 | M60.2 | Unknown | + | — |
| 177920_at | D1086 | D1086.3 | Unknown | − | — |
| 178149_s_at | T07C4 | T07C4.4 | spp-1 | − | — |
| 178409_s_at | F58B3 | F58B3.3 | Lys-6 | − | — |
| 180913_at | F32A5 | F32A5.5a, F32A5.5b | aqp-1 | + | — |
| 181156_at | T08A9 | T08A9.8 | spp-4 | + | — |
| 182330_s_at | Y37A1A | Y37A1A.2 | Unknown | + | — |
| 183010_s_at | C24B9 | C24B9.3a, C24B9.3b | Von Willebrand factor A domain | + | COI12A1, TTBK2 |
| 183392_at | C02B8 | K07E3.1 | Similar to hemogglutinin domain | + | Dentin |
| | | | | | sialophosphoprotein |
| | | | | | precursor |
| 184079_at | R09B5 | R09B5.4 | Iron transporter domain | + | — |
| 184662_at | Y46G5A | Y46G5A.29 | ShTK domain | + | Notch 2 |
| 187247_at | F59B10 | F59B10.5 | Unknown | − | — |
| 187996_s_at | F28H7 | F28H7.3 | Lipase domain | − | — |
| 188037_at | ZK377 | ZK377.1 | wrt-6 | + | Indian hedgehog |
| | | | | | protein precursor |
| 188078_at | F46B6 | F46B6.8 | Triglyceride lipase domain | + | — |
| 188802_at | F54D5 | F54D5.8 | dnj-13 | + | — |
| 189732_at | F49E12 | F49E12.9 | Sterol desaturase domain | + | — |
| 190987_at | R13H4 | R13H4.3 | Histidine acid phosphatase domain | + | — |
| 192144_s_at | ZK455 | ZK455.4 | asm-2 | + | SMPD1 |
| 193836_s_at | W04E12 | W04E12.8 | C-type lectin domain | + | mannose receptor, |
| | | | | | C type 2 |
| | | 23 | | 15 | 6 |

| Affy ID | TMDs | Transporters | Metal/Zinc finger | Nucleic acid | Known |
|---|---|---|---|---|---|
| >2(all inc) | | | | | |
| 176851_at | | | | | |
| 180150_at | | | | | |
| 184035_at | | | | | |
| 189419_at | 1_C | | | | |
| 176681_at | | | | | |
| 179098_at | | | | | |
| 183117_at | | | | | |
| 183379_at | | | | | |
| 183676_s_at | | | | | |
| 185399_at | | ptn transport in | | | |
| | | yeast | | | |
| 186383_at | 1_N | | | | |
| 187962_at | 1_N | | | | |
| 190134_s_at | 1_N | | | | |
| 190693_at | 2 | | | | |
| 191418_at | 1_C | UDP transferase | | | |
| 180973_at | | | heme | | |
| | 6 | 2 | 1 | | 0 |
| 1.6-2(all inc) | | | | | |
| 173836_at | | | hemopexin repeat | | toh-1: tollish |
| 177024_at | 1_N | | | + | |
| 182573_at | | | | | |
| 184313_s_at | | | | | |
| 184399_at | | | | | |
| 185343_at | | | | | |
| 185425_at | | | | | |
| 187662_at | 1_N | | | | |
| 188028_at | | | | | grd-6: hedgehog-like family |
| 192059_at | | sugar transporter | | | |
| | | 10 family | | | |
| 194067_at | | | | | |
| | 3 | 1 | 1 | 1 | 2 |
| >2(All Dec) | | | | | |
| 175239_at | | | | | |
| 186519_at | | | | | |
| 183666_at | 1_N | | | | |
| 183724_at | 1_N | | | | |
| 188391_at | 1_N | | | + | bli-2 |
| 189482_s_at | 1_N | | | | |
| 171941_s_at | | | | | |
| 172400_x_at | | | | | |
| 172971_s_at | 1_M | | | + | |
| 176395_at | | | | | |
| 177613_at | 1_N | | | | |
| 177812_at | | 12 glpT transporter | | | |
| 178087_s_at | | | | | lys-5 |
| 180616_at | | | | | |
| 180706_at | 1_N | | | | |
| 181502_s_at | 8 | | | | |
| 181520_at | 11 | | | | |
| 183273_at | 1_N | | | | |
| 185242_at | | | | | |
| 188495_at | 9 | phosphate | | | |
| | | transport | | | |
| 189595_s_at | 1_N | | | | |
| 190958_s_at | | | | | |
| 191882_at | 1_C | | | | |
| 193588_s_at | | | | | |
| | 14 | 2 | 0 | 2 | 2 |
| 1.6-2(all dec) | | | | | |
| 180880_at | 1_N | | | | |
| 181819_s_at | | | | | |
| 181902_at | | | | | |
| 187382_at | 1_N | | | | |
| 187846_at | | | | | |
| 189864_s_at | 1_N | | | | |
| 189975_at | | | | | |
| 193034_at | | | | | |
| | 3 | | | | |
| >2 (De4/In500) | | | | | |
| 180562_at | | | | | |
| 171932_x_at | 1_N | | | | |
| 172184_x_at | | | | | |
| 178297_at | | | zinc finger | | |
| 178843_at | | | | | |
| 179424_at | | | | | |
| 182970_at | | | | | |
| 183527_s_at | | | | | |
| 187964_at | | | | | |
| 188441_at | | | | | |
| 192509_at | | | | | |
| | 1 | | 1 | | 0 |
| >2 (In 4/De500) | | | | | |
| 172134_x_at | | lipid transport ptn | | vit-4 | |
| 173411_s_at | | lipid transport ptn | | vit-4 | |
| 175993_at | | | | | |
| 184144_at | | | | | |
| 188335_at | 1_N | | | | |
| 188947_at | 1_N | | | | |
| 189227_at | | | | | |
| 189660_at | 1_N | | Calcium bind | | |
| 189911_s_at | 1_N | | | | |
| 190619_at | | | | | |
| 180752_s_at | | Solute carrier, Zinc | | | |
| | | transporter | | | |
| 194239_x_at | | | | vit-3 | |
| | 4 | 3 | 1 | | 3 |
| 1.6 to 2 | | | | | |
| 177428_at | | 3 Ctr family | | | |
| 188245_at | 1_N | | | | |
| | 2 | 1 | 0 | 0 | 0 |
| >2(NC4/In500) | | | | | |
| 177816_at | 4 | | | | |
| 178017_s_at | | | | | |
| 179933_at | | | | | |
| 180315_at | 4 | | | | |
| 180727_at | | | | | |
| 181099_at | 1_C | | | | |
| 181946_at | | | | | |
| 183020_at | | | | | |
| 183526_at | | | | | |
| 183665_s_at | | | | | |
| 183850_at | | | | | |
| 184116_s_at | | | | | |
| 184352_at | | | | | |
| 184624_s_at | | | | | |
| 184707_s_at | | | | | |
| 185145_at | | | | | |
| 188106_at | | | Iron-sulphur bind | | |
| 188465_s_at | 1_N | | | | |
| 188987_at | | | | | |
| 189971_at | | | | | |
| 190139_s_at | 1_N | | | | |
| 190830_at | | | | | |
| 191502_at | 1_C | | | | |
| 191568_at | | | | | |
| 192076_at | 4 | MIP family | | | |
| 193007_s_at | 11 | ABC transporter | | | pgp-1 |
| 194063_at | | | | | |
| | 8 | 2 | 1 | | 1 |
| 1.5-2(NC4/In500) | | | | | |
| 172069_x_at | | | | | cpr-3-(Cysteine |
| | | | | | PRotease related) |
| 172503_x_at | | | | Iron-sulphur bind | |
| 172769_x_at | | | | | |
| 173090_s_at | | | | | |
| 175170_s_at | | | | | |
| 175870_s_at | | | | | |
| 176007_at | | 5 | | | |
| 176026_at | | | | | |
| 176360_at | | | | Zinc finger | |
| 176756_s_at | | | | | |
| 176826_at | | | | | |
| 177059_at | | | | | |
| 177187_at | | | | | |
| 177219_at | | 5 | | | |
| 177693_at | 1_N | | | | |
| 177827_at | | | | | |
| 178128_at | | | | | |
| 178403_at | | | | | |
| 179682_at | | | | | |
| 179989_at | | | | | |
| 180379_s_at | | | | | |
| 180410_at | | | | | |
| 180642_at | | | Zinc finger | + | |
| 180810_at | 7 | | | | |
| 181459_at | 4 | | | | |
| 182106_at | 1_N | | | | |
| 182276_at | | | | | |
| 182279_at | | | | | |
| 182712_at | | | | | |
| 182790_at | | | | | |
| 183702_at | | | | | |
| 183867_at | | | | | |
| 186413_at | 1_C | | | | |
| 186528_s_at | | | Zinc finger | | |
| 186799_at | | | | | |
| 186832_at | | | | | |
| 187548_s_at | | | | | |
| 188589_at | 1_N | | Transferrin bind | + | |
| 188648_at | | | | | |
| 189179_at | | | | | hil-1 - (Histone H1 Like) |
| 189325_at | | | | | |
| 189473_at | 15 | abt-4; ABC | | | |
| | | transporter | | | |
| 190156_s_at | | | | | |
| 190301_s_at | 10 | ammonium | | | |
| | | transporter | | | |
| 190413_at | | | | | |
| 190899_at | | | | | |
| 190959_s_at | | | | | |
| 191298_s_at | 1_N | | | | |
| 191336_at | 7 | | | | |
| 191406_at | | multicopper | | | |
| | | oxidases | | | |
| 191541_at | 1_N | | | | |
| 191970_at | | | Zinc finger | | odd-2 - (Drosophila ODD- |
| | | | | | skipped-like) |
| 192249_at | | | | | |
| 192559_s_at | | | | | |
| 192596_s_at | | | | | |
| 192610_at | | | | | nas-12 - (Nematode |
| | | | | | AStacin protease) |
| 192628_at | 4_C | | | | glc-1 - (Glutamate-gated |
| | | | | | ChLoride channel) |
| 193165_at | 12 | ammonium | | | rhr-1 - (RH (Rhesus) |
| | | transporter RHBG | | | antigen Related) |
| 193926_at | | | Zinc finger | | |
| | 15 | 4 | 7 | 2 | 6 |
| >2(NC4/De500) | | | | | |
| 176230_at | | | | | |
| 177532_at | 1_M | | | | |
| 179226_at | | | | | |
| 180997_at | | | | | |
| 183455_at | 1_N | | | | |
| 189366_at | | | | | |
| 190080_at | | | | | |
| 190962_s_at | | | | | |
| 193584_s_at | | | | | cyn-2 |
| | 2 | | | | 1 |
| 1.6-2(NC4/De500) | | | | | |
| 172904_x_at | | | | | |
| 173632_s_at | 1_N | | | | |
| 173650_s_at | 1_N | | | + | |
| 174152_s_at | 1_N | | | | bli-1 - (BLIstered cuticle) |
| 174273_at | | | | | |
| 174960_at | 1_N | | | | |
| 175898_at | | | | | |
| 176419_at | | | | | |
| 176461_s_at | | | | | |
| 177065_at | | lipid transport ptn | | | vit-1 - (VITellogenin |
| | | | | | structural genes (yolk |
| | | | | | protein genes)) |
| 177729_at | | | | | |
| 178189_at | | | | | |
| 178243_s_at | | | | | |
| 178439_s_at | | | | | |
| 178759_at | | | | | |
| 178929_at | 1_C | | | | |
| 179199_at | | | | | |
| 180462_at | 1_N | | | | |
| 181331_s_at | | | | | |
| 181833_at | 2_C | | | | |
| 182078_at | 4 | | | | |
| 182498_at | | | | | |
| 183232_s_at | 3 | | | | |
| 183713_at | 2 | | | | |
| 184740_at | 2 | | | | |
| 184839_at | 6 | | | | |
| 184864_at | 1_N | | | | |
| 186349_at | 2 | | | | |
| 186974_s_at | | | | | |
| 187612_s_at | | | | | |
| 188128_at | | | | | |
| 188849_at | | | | | |
| 188917_s_at | | | zinc finger | | |
| 189208_at | | | | | |
| 189762_at | 1_N | | | + | |
| 189921_at | | | | | |
| 190094_s_at | | | | | |
| 190585_at | | | | | |
| 191303_at | | | | | |
| 192175_at | 1_C | | | | |
| 192786_s_at | | | | | |
| 192819_at | | | zinc finger | | |
| 193127_s_at | | | | | dao-3 - (Dauer or Aging |
| | | | | | adult Overexpression) |
| 193885_at | 1_C | | Calcium bind | | |
| 194086_at | | | | | |
| | 17 | 1 | 3 | 2 | 3 |
| >2(In4/NC500) | | | | | |
| 174713_at | | | | | |
| 175810_at | | | zinc finger | | pos-1 |
| 177544_at | 2 | | | | |
| 179900_s_at | | | | | |
| 185275_at | 1_N | | | | cdr-5 |
| 186182_s_at | 4 | | | | |
| 186232_s_at | | | zinc finger | | |
| 189561_at | 1_N | | | | |
| 189671_at | 1_N | | | | |
| 192163_at | 1_N | | | | col-19 |
| 173316_s_at | | | | | |
| 173647_s_at | 1_N | | | | |
| 174283_s_at | | | | | |
| 174964_at | | | | | |
| 175010_s_at | 1_N | | | | |
| 175377_at | | | | | |
| 176004_at | | | zinc finger | | |
| 177253_at | | | | | |
| 177389_at | 2 | | | | |
| 177820_at | | | | | |
| 178775_at | | | | + | |
| 179525_at | 3 | | | | |
| 179805_s_at | | | | | |
| 180173_at | | | | | |
| 180306_at | 1_N | trypsin inhibitor like | | | |
| | | Cys rich domain | | | |
| 180361_at | | | | | |
| 180676_at | | | | | |
| 182492_at | | | | | |
| 183721_at | | | | | |
| 185762_at | | | | | |
| 186231_at | | | | | |
| 188406_s_at | | | | | col-8 |
| 188456_at | 1_N | | | | |
| 190530_s_at | 1_N | | | | col-20 |
| 192345_s_at | | | | | |
| | 13 | 1 | 3 | 1 | 5 |
| 1.6-2(In4/NC500) | | | | | |
| 172443_x_at | | | | | |
| 175102_at | 1_N | GABA receptor | | | |
| 177489_at | | | | | |
| 178259_at | | | | | |
| 180540_at | | | | + | |
| 182415_s_at | | | | | |
| 182599_s_at | | | | | |
| 182628_s_at | | | | | |
| 183115_at | | | | | |
| 183187_at | | | | | |
| 184393_at | | | | + | |
| 184943_s_at | | | | + | |
| 185954_at | | | | | |
| 185964_at | | | | | |
| 187358_at | | | zinc finger | | |
| 188747_at | 1_N | | | | |
| 189178_at | | | | | |
| 189953_at | | | Hemopexin repeat | | nas-20 - (Nematode |
| | | | | | AStacin protease) |
| 191251_at | 1_C | | | | |
| 191470_s_at | | | | | |
| 192956_at | | | | | |
| | 3 | 1 | 2 | 3 | 1 |
| >2(De4/NC500) | | | | | |
| 192194_s_at | | | | | |
| 174393_at | | | | | |
| 179360_s_at | | | | | |
| 179879_at | | 2 | | | |
| | 1 | | | | 0 |
| 1.6-2(De4/NC500) | | | | | |
| 173051_s_at | | | | | |
| 176474_at | | | | + | |
| 176864_at | | | | | |
| 177375_at | | | | | |
| 177920_at | | | | | |
| 178149_s_at | | | | | spp-1: saposin-like protein |
| | | | | | family |
| 178409_s_at | | | | | |
| 180913_at | | 6 MIP family | | | aqp-1 - (AQuaPorin or |
| | | | | | aquaglyceroporin related) |
| | | | | | spp-4 - (SaPosin-like |
| | | | | | Protein family) |
| 181156_at | 1_N | | | | |
| 182330_s_at | | H+ transporting 2-12 | | | |
| | | sector ATPase | | | |
| 183010_s_at | | | | | |
| 183392_at | 4_N | | | | |
| 184079_at | | solute carrier | | | |
| | | 9 protein | | | |
| 184662_at | | | Calcium bind | | |
| 187247_at | | | | | |
| 187996_s_at | 1_N | | | | |
| 188037_at | | | | | wt-6 - (WaRThog |
| | | | | | (hedgehog-like family)) |
| 188078_at | | | | | |
| 188802_at | | | | | |
| 189732_at | 2_N | | | | |
| 190987_at | | | | | |
| 192144_s_at | | | | | |
| 193836_s_at | | | | | |
| | 7 | 3 | 1 | 1 | 4 |

For Table 2: All collagen genes (27 genes) have been removed from the table.

| Affy ID | Wormbase | Wormbase | Worm Profile | humans | Knownhuman |
|---|---|---|---|---|---|
| >2(all inc) | | | | | |
| 176851_at | Y40B10A | Y40B10A.6 | O-methyl transferase domain | + | — |
| 180150_at | F54E2 | F54E2.1 | Unknown | − | — |
| 184035_at | M02F4 | M02F4.7 | C-type lectin domain | + | — |
| 189419_at | F15B9 | F15B9.6 | Phospholipase domain | − | — |
| 176681_at | F35B3 | F35B3.4 | Fibronectin domain | − | — |
| 179098_at | T19C9 | T19C9.8 | Unknown | − | — |
| 183117_at | ZK742 | ZK742.3 | NADH: Flavin oxireductase domain | − | — |
| 183379_at | K01D12 | K01D12.9 | Unknown | − | — |
| 183676_s_at | F08F8 | F08F8.5 | Unknown | + | — |
| 185399_at | Y75B8A | Y75B8A.28 | Unknown | − | — |
| 187962_at | K02E2 | K02E2.4 | ins-35 | − | — |
| 190693_at | K08B4 | K08B4.3 | UDP-glucuronosyl and UDP-glucosyl | + | — |
| | | | transferase domain | | |
| 191418_at | ZC443 | ZC443.6 | UDP-glucuronosyl and UDP-glucosyl | + | — |
| | | | transferase domain | | |
| 180973_at | F49F1 | F49F1.6 | ShTK domain | + | mucin-2 |
| | | 14 | | 6 | 1 |
| 1.6-2(all inc) | | | | | |
| 173836_at | T24A11 | T24A11.3 | Toh-1 | + | — |
| 182573_at | T05B4 | T05B4.3 | ShTK domain | − | — |
| 184313_s_at | K11H12 | K11H12.4 | Unknown | − | — |
| 184399_at | Y26D4A | Y26D4A.10, Y26D4A.11 | Unknown | − | — |
| 185343_at | T28C12 | T28C12.6 | Unknown | − | — |
| 185425_at | Y38E10A | Y38E10A.5 | C-type lectin domain | + | — |
| 187662_at | F32H5 | F32H5.3a, F32H5.3b | Unknown | − | — |
| 188028_at | F07C3 | T18H9.1 | grd-6 | + | mucin-2 |
| 192059_at | Y51A2D | Y51A2D.4 | MFS domain | + | — |
| 194067_at | F35C5 | F35C5.9 | C-type lectin domain | − | — |
| | | 10 | | 4 | 1 |
| >2 All Dec. | | | | | |
| 175239_at | F15E11 | F15E11.15 | Unknown | − | — |
| 186519_at | F15E11 | F15E11.12, F15E11.15 | Unknown | − | — |
| 183724_at | Y11D7A | Y11D7A.5 | Unknown | − | — |
| 171941_s_at | F44E5 | F44E5.5 | Hsp70 domain | + | — |
| 172400_x_at | F22A3 | F22A3.6a, F22A3.6b | Destabliase domain | − | — |
| 172971_s_at | Y54G2A | Y54G2A.11a, Y54G2A.11b | Myb domain | + | — |
| 176395_at | Y71G12B | Y71G12B.18 | Unknown | − | — |
| 177613_at | F57G8 | F57G8.7 | Unknown | − | — |
| 177812_at | F10C2 | F10C2.7 | MFS domain | + | NA/PI-4 |
| 178087_s_at | F58B3 | F58B3.2 | Lys-5 | − | — |
| 180616_at | T22B7 | T22B7.3 | Amidinotransferase domain | − | — |
| 180706_at | K06H6 | K06H6.2 | Unknown | − | — |
| 181502_s_at | W07A12 | W07A12.6 | Acyltransferase domain | − | — |
| 181520_at | W07A12 | W07A12.7 | Acyltransferase domain | − | — |
| 183273_at | C14C6 | C14C6.3 | Glycosyl transferase domain | − | — |
| 185242_at | Y105C5B | Y105C5B.7 | Calcineurin like phosphoesterase | + | — |
| | | | domain | | |
| 188495_at | F09G2 | F09G2.3 | Phosphate transporter domain | + | leukemia virus |
| | | | | | receptor 1 |
| 189595_s_at | K10C2 | K10C2.3 | Aspartyl protease domain | + | — |
| 190958_s_at | F44E5 | F44E5.4 | Hsp70 domain | + | HSP70A8 |
| 191882_at | F47C10 | F47C10.6 | UDP-glucuronosyl and UDP-glucosyl | + | — |
| | | | transferase domain | | |
| 193588_s_at | F28D1 | F28D1.5 | thn-2 | − | — |
| | | 21 | | 8 | 3 |
| 1.6-2(alt dec) | | | | | |
| 180880_at | K06H6 | K06H6.1 | Unknown | − | — |
| 181819_s_at | Y37D8A | Y37D8A.4 | SH2 domain | + | — |
| 181902_at | Y43D4A | Y43D4A.5 | Unknown | + | MUC-1 |
| 187382_at | F55C10 | F55C10.4 | Unknown | − | — |
| 187846_at | W03F11 | W03F11.5a, W03F11.5b | Unknown | − | — |
| 189975_at | F08H9 | F08H9.5 | C-type lectin domain | + | — |
| 193034_at | F56H6 | F56H6.5 | GDP-mannose 4,6-dehydratase domain | + | — |
| | | 7 | | 4 | 1 |
| >2 (De4/In500) | | | | | |
| 180562_at | B0218 | B0218.8 | C-type lectin domain | + | — |
| 171932_x_at | F09C8 | F09C8.1 | Phospholipase domain | + | — |
| 172184_x_at | Y46C8AL | Y46C8AL.2 | Unknown | + | — |
| 178297_at | T24B8 | T24B8.5 | ShTK domain | − | — |
| 178843_at | F08G5 | F08G5.6 | Unknown | − | — |
| 179424_at | F27C8 | F27C8.4 | spp-18 | − | — |
| 182970_at | K10D11 | F55G11.4 | Unknown | − | — |
| 183527_s_at | C14C6 | C14C6.5 | ShTK domain | − | — |
| 187964_at | F54F3 | F54F3.3 | Lipase domain | + | — |
| 188441_at | F21F8 | F21F8.4 | Aspartyl protease domain | + | gastric cathepsin E |
| 192509_at | ZK666 | ZK666.6 | C-type lectin domain | − | — |
| | | 11 | | 5 | 1 |
| >2 (in 4/De500) | | | | | |
| 172134_x_at | F56B6 | F59D8.2 | Vit-4 | + | Apolipoprotein B- |
| | | K07H8.6a, K07H8.6b, K07H | | | 100 |
| 173411_s_at | K07H8 | 8.6c | Vit-6 | + | — |
| 175993_at | C29E4 | C29E4.7 | GST domain | + | — |
| 188947_at | T09F5 | T09F5.9 | C-type lectin domain | + | — |
| 189227_at | W07B8 | W07B8.1 | Cystein protease domain | + | Cathepsin B1 |
| 189660_at | F59D6 | F59D6.3 | Aspartyl protease domain | + | — |
| 190619_at | C15C8 | C15C8.3 | Aspartyl protease domain | + | gastric cathepsin E |
| 180752_s_at | D1054 | D1054.10 | Unknown | − | |
| 194239_x_at | F59D8 | F59D8.1 | Vit-3 | + | Apolipoprotein B- |
| | | | | | 100 |
| | | 9 | | 8 | 4 |
| 1.6 to 2 | | | | | |
| 177428_at | F58G6 | F58G6.3, F58G6.7 | Ctr domain | + | human Ctr1 |
| | | 1 | | 1 | 1 |
| >2(NC4/In500) | | | | | |
| 177816_at | F49H6 | F49H6.3 | Unknown | − | — |
| 178017_s_at | F01D5 | F01D5.1 | ShTK domain | + | — |
| 179933_at | F39E9 | F39E9.1 | Unknown | − | — |
| 180315_at | F44G3 | F44G3.10 | Unknown | − | — |
| 180727_at | F49F1 | F49F1.5 | ShTK domain | − | — |
| 181099_at | F54C1 | F54C1.1 | UDP-glucuronosyl and UDP-glucosyl | + | — |
| | | | transferase domain | | |
| 181946_at | C03H5 | C03H5.1 | C-type lectin domain | + | MMR |
| 183020_at | F48G7 | F48G7.8 | ShTK domain | − | — |
| 183526_at | C14C6 | C14C6.5 | ShTK domain | − | — |
| 183665_s_at | F48G7 | F48G7.5 | ShTK domain | − | — |
| 183850_at | Y48E1B | Y48E1B.8 | Unknown | − | — |
| 184116_s_at | T05E12 | T05E12.6 | Unknown | − | — |
| 184352_at | C17H12 | C17H12.6 | Unknown | − | — |
| 184624_s_at | C25H3 | C25H3.10, C25H3.10a, C25H3.10b | F-box domain | − | — |
| 184707_s_at | C32H11 | C32H11.10 | dod-21 | − | — |
| 185145_at | Y46D2A | Y46D2A.2 | Unknown | − | — |
| 188106_at | T01C3 | T01C3.4 | Lipase domain | − | — |
| 188465_s_at | R09D1 | R09D1.8 | glycosyl hydrolases domain | + | — |
| 188987_at | F20G2 | F20G2.1 | Short chain dehydrogenase domain | + | — |
| 189971_at | F01G10 | F01G10.3 | ech-9 | + | PBFE |
| | | | | | mannose receptor, |
| 190139_s_at | E03H4 | E03H4.10 | CUB domain | + | C type 2 |
| 190830_at | T21C9 | T21C9.8 | Transthyretin like domain | − | — |
| 191502_at | ZC443 | ZC443.5 | UDP-glucuronosyl and UDP-glucosyl | + | — |
| | | | transferase domain | | |
| 191568_at | C13D9 | C13D9.9 | UDP-glucuronosyl and UDP-glucosyl | + | — |
| | | | transferase domain | | |
| 192076_at | K02G10 | K02G10.7a, K02G10.7b | aqp-8 | + | — |
| 193007_s_at | K08E7 | K08E7.9 | pgp-1 | + | P-glycoprotein |
| 194063_at | F35C5 | F35C5.7 | C-type lectin domain | − | — |
| | | 27 | | 11 | 4 |
| 1.6-2(NC4/In500) | | | | | |
| 172069_x_at | T10H4 | T10H4.12 | cpr-3 | + | Cathepsin B1 |
| 172503_x_at | W08E12 | W08E12.3 | 2Fe-2S-ferredoxin domain | + | — |
| 172769_x_at | F44C8 | F44C8.1 | cyp-33C4 | + | — |
| 173090_s_at | F35C5 | F35C5.8 | C-type lectin domain | + | C-type lectin |
| 175170_s_at | ZK6 | ZK6.10 | dod-19 | − | — |
| 175870_s_at | Y34F4 | Y34F4.4 | Unknown | − | — |
| 176007_at | Y50D4A | Y50D4A.1 | Ribosome protein S2 domain | + | — |
| 176026_at | C06E1 | C06E1.3 | Unknown | + | — |
| 176360_at | R13A5 | R13A5.10 | Cytidine deaminase domain | + | — |
| 176756_s_at | Y65B4A | F56A6.1a, F56A6.1b | Piwi domain | + | — |
| 176826_at | Y40B10A | Y40B10A.2 | O-methyl transferase domain | + | — |
| 177059_at | Y46C8AL | Y46C8AL.5 | C-type lectin domain | − | — |
| 177187_at | Y46C8AR | Y46C8AR.1 | C-type lectin domain | − | — |
| 177219_at | M28 | M28.8 | Glutamine amidotransferase domain | − | — |
| 177693_at | R03G8 | R03G8.3 | Unknown | − | — |
| 177827_at | T19C4 | T19C4.5 | Unknown | − | — |
| 178128_at | F01D5 | F01D5.3 | ShTK domain | − | — |
| 178403_at | F10A3 | F10A3.2 | FTH domain | − | — |
| 179682_at | C31H5 | C31H5.6 | Acyl-CoA thioester hyrolase domain | + | — |
| 179989_at | F36G9 | F36G9.14 | FTH domain | − | — |
| 180379_s_at | C17F4 | C17F4.7 | Unknown | − | — |
| 180410_at | Y39G8B | Y39G8B.7 | ShTK domain | − | — |
| 180642_at | F53A9 | F53A9.2 | Peptidase M domain | + | — |
| 180810_at | R10D12 | R10D12.9 | MtN3/saliva family domain | + | — |
| 181459_at | Y102A5C | F49H6.13 | Unknown | − | — |
| 182106_at | C34H4 | C34H4.1 | Unknown | − | — |
| 182276_at | T10B10 | T10B10.4, T10B10.4a | Unknown | − | — |
| 182279_at | Y47H9C | Y47H9C.1 | Unknown | − | — |
| 182712_at | C49G7 | C49G7.4 | ShTk domain | − | — |
| 182790_at | F10G2 | F10G2.3 | C-type lectin domain | + | — |
| 183702_at | K05B2 | K05B2.4 | Acyl-CoA thioester hyrolase domain | + | — |
| 183867_at | C45E5 | C45E5.4 | Unknown | − | — |
| 186413_at | H20E11 | H20E11.1a, H20E11.1b | Unknown | − | — |
| 186528_s_at | F46E10 | F46E10.11 | Unknown | + | — |
| 186799_at | Y105C5B | Y105C5B.15 | Calcineurin like phosphoesterase | + | — |
| | | | domain | | |
| 186832_at | F27E5 | F27E5.1 | Acid ceramidase | + | Acid ceramidase |
| | | | | | precursor |
| 187548_s_at | C32D5 | C32D5.6 | Unknown | + | — |
| 188648_at | C45B2 | C45B2.5 | Glutamine synthase domain | + | — |
| 189179_at | C30G7 | C30G7.1 | hil-1 | + | — |
| 189325_at | F02D8 | F02D8.4 | Zinc carboxylpeptidase domain | + | PCPB protein |
| 189473_at | Y39D8C | Y39D8C.1 | abt-4 | + | human ABCA3 |
| 190156_s_at | C48B4 | C48B4.1 | Acyl-CoA dehydrogenase domain | + | Acyl-coenzyme A |
| | | | | | isoform b |
| 190301_s_at | C05E11 | C05E11.5 | amt-4 | + | — |
| 190413_at | F25G6 | F25G6.6 | nrs-2 | + | Asparagine |
| | | | | | synthetase |
| 190899_at | F21H7 | F21H7.1 | GST-22 | + | Prostaglandin-D |
| | | | | | synthase |
| 190959_s_at | F37B1 | F37B1.5 | GST-16 | + | Prostaglandin-D |
| | | | | | synthase |
| 191298_s_at | F23B2 | F23B2.12 | pcp-2 | + | — |
| 191336_at | K06C4 | K06C4.8 | rhodopsin domain | + | — |
| 191406_at | F21D5 | F2105.3 | Multicopper oxidase domain | − | — |
| 191541_at | C54D10 | C54D10.1 | GST domain | + | — |
| 191970_at | C34H3 | C34H3.2 | odd-2 | + | — |
| 192249_at | F22A3 | F22A3.1 | SAM/pointed domain | + | — |
| 192559_s_at | F35C5 | F35C5.8 | C-type lectin domain | + | CLECSF6 |
| 192596_s_at | T03F7 | T03F7.7a, T03F7.7b | CRAL/TRIO domain | + | SEC14-like 2 |
| 192610_at | C24F3 | C24F3.3 | nas-12 | + | — |
| 192628_at | F11A5 | F11A5.10 | glc-1 | + | — |
| 193165_at | F08F3 | F08F3.3 | rhr-1 | + | — |
| 193926_at | Y48A6B | Y48A6B.7 | Cytidine deaminase domain | + | — |
| | | 58 | | 38 | 11 |
| >2(NC4/De500) | | | | | |
| 176230_at | Y71H2AM | Y71H2AM.16 | Histidine acid phosphatase domain | + | Prostatic acid |
| | | | | | phosphatase |
| 177532_at | F22B5 | F22B5.4 | Unknown | − | — |
| 179226_at | C06B3 | C06B3.7 | Unknown | − | — |
| 180997_at | C34D4 | C34D4.3 | MSP domain | + | — |
| 183455_at | F26B1 | F26B1.1 | Unknown | + | — |
| 189366_at | K07C6 | K07C6.4 | cyp-35B1 | + | cytochrome P450, |
| | | | | | 2U |
| 190080_at | F26C11 | F26C11.1 | Histidine acid phosphatase domain | + | — |
| 190962_s_at | F37B1 | F37B1.8 | GST-19 | + | Prostaglandin-D |
| | | | | | synthase |
| 193584_s_at | ZK520 | ZK520.5 | cyn-2 | + | — |
| | | 9 | | 7 | 3 |
| 1.6-2(NG4/De500) | | | | | |
| 172904_x_at | K05F1 | K05F1.7 | msp-63 | + | — |
| 174273_at | F01G10 | F01G10.9 | Unknown | + | — |
| 175898_at | ZK105 | ZK105.1 | Unknown | − | — |
| 176419_at | Y71G12B | Y71G12B.17 | Phosphatidylinositol transfer protein | + | phosphatidylinositol |
| | | | domain | | transfer protein |
| 176461_s_at | Y59H11AM | Y59H11AM.3 | Rhodanese-like domain | + | — |
| 177065_at | K09F5 | K09F5.2 | Vit-1 | + | — |
| 177729_at | E01G4 | E01G4.6 | Phospholipase domain | + | — |
| 178189_at | F28C6 | F28C6.5 | Unknown | − | — |
| 178243_s_at | F32B6 | F32B6.4 | Unknown | − | — |
| 178439_s_at | F17C8 | F17C8.7 | Unknown | − | — |
| 178759_at | F59C6 | F59C6.6 | nlp-4 | − | — |
| 178929_at | T03F7 | F47G9.3 | Zona pellucida-like domain | − | — |
| 179199_at | T09F5 | T09F5.1 | Galactosyltransferase domain | + | — |
| 180462_at | C07E3 | C07E3.10 | Unknown | − | — |
| 181331_s_at | K09C6 | K09C6.8 | Unknown | + | — |
| 181833_at | K01A2 | K01A2.4 | Unknown | + | — |
| 182078_at | T10E9 | T10E9.8 | Unknown | − | — |
| 182498_at | H25K10 | H25K10.1 | Calcineurin like phosphoesterase | + | — |
| | | | domain | | |
| 183232_s_at | K01A2 | K01A2.3 | Unknown | − | — |
| 183713_at | C24G7 | C24G7.2 | Amiloride-sensitive sodium channel | + | — |
| | | | domain | | |
| 184740_at | C33F10 | C33F10.1 | Unknown | − | — |
| 184839_at | T06C10 | C55F2.1b | ALCARFT/IMPCHase bienzyme domain | + | — |
| 184864_at | C08F11 | C08F11.11 | Unknown | − | — |
| 187612_s_at | F47D12 | F47D12.7 | Kelch motif domain | + | — |
| 188128_at | C50B6 | C50B6.7 | Alpha-amylase domain | + | — |
| 188849_at | C56E6 | C56E6.2 | Ras domain | + | — |
| 188917_s_at | F26H11 | F26H11.2c, F26H11.2d | DDT domain | + | Fetal Alzheimer |
| | | | | | antigen isoForm 1 |
| 189208_at | F38B6 | F38B6.4 | Phosphoribosylglycinamide synthase | + | phosphoribosyl- |
| | | | domain | | glycinamide |
| | | | | | formyltransferase |
| 189921_at | C05C10 | C05C10.4 | Histidine acid phosphatase domain | + | — |
| 190094_s_at | M02D8 | M02D8.4a, M02D8.4b, M02 | Asparagine synthase domain | + | Asparagine |
| | | D8.4c | | | synthetase |
| 190585_at | ZK970 | ZK970.7 | Unknown | − | — |
| 191303_at | Y53F4B | Y53F4B.32 | GST-29 | + | |
| 192175_at | C27D6 | C27D6.3 | Unknown | + | PARP1 protein |
| 192786_s_at | B0286 | B0286.3 | SAICAR synthase domain | + | — |
| 192819_at | K10C3 | Y67A6A.2 | nhr-62 | + | HNF4A |
| 193127_s_at | K07E3 | K07E3.3 | dao-3 | + | C-1- |
| | | | | | tetrahydrofolate |
| | | | | | synthase |
| 193885_at | B0393 | B0393.5 | EGF-like domain | + | Fibrillin 1 |
| 194086_at | W01F3 | W01F3.3 | Trypsin inhibitor domain | + | — |
| | | 38 | | 26 | 8 |
| >2( [symbol] n4/NC600) | | | | | |
| 174713_at | F14F4 | F14F4.3a | mrp-5 | − | — |
| 175810_at | F52E1 | F52E1.1 | pos-1 | + | — |
| 177544_at | F58E6 | F58E6.7, F58E6.11 | Unknown | − | — |
| 179900_s_at | C44B7 | C44B7.5 | Cyt b-561/ferric reductase domain | − | — |
| 185275_at | C54D10 | K01D12.14 | cdr-5 | + | — |
| 186182_s_at | R02E12 | R02E12.6 | Unknown | + | — |
| 186232_s_at | C44B12 | C44B12.1 | Unknown | − | — |
| 189671_at | C50H11 | C50H11.15 | cyp-33C9 | + | — |
| 173316_s_at | Y62H9A | Y62H9A.6 | Unknown | − | — |
| 174283_s_at | F38B6 | F38B6.4 | Phosphoribosylglycinamide synthase | + | — |
| | | | domain | | |
| 174964_at | K02B9 | K02B9.1 | Unknown | − | — |
| 175010_s_at | F57C2 | F57C2.4 | Unknown | − | — |
| 175377_at | ZK813 | ZK813.1 | Unknown | − | — |
| 176004_at | M02H5 | M02H5.4 | Zinc finger domain | + | HNF4A |
| 177253_at | R186 | R186.1 | Unknown | + | — |
| 177389_at | F58E6 | F58E6.8 | Unknown | − | — |
| 177820_at | ZC373 | ZC373.2 | Unknown | − | — |
| 178775_at | C01G6 | C01G6.3 | Unknown | − | — |
| 179525_at | F36H1 | F36H1.5 | Unknown | − | — |
| 179805_s_at | T04G9 | T04G9.7 | Unknown | − | — |
| 180173_at | Y62H9A | Y62H9A.4 | Unknown | − | — |
| 180306_at | C10G8 | C10G8.4 | Trypsin inhibitor domain | + | — |
| 180361_at | F42G2 | F42G2.2 | FTH domain | − | — |
| 180676_at | ZK813 | ZK813.1 | Chorion domain | + | — |
| 182492_at | C16C4 | C16C4.4 | MATH domain | − | — |
| 183721_at | F17E9 | F17E9.4 | Unknown | − | — |
| 185762_at | F47C10 | F47C10.2 | BTB domain | + | — |
| 186231_at | C44B12 | C44B12.1 | Unknown | − | |
| 192345_s_at | F32H5 | F32H5.1 | Cystein protease domain | + | Cathepsin B1 |
| | | 29 | | 11 | 2 |
| [symbol] | | | | | |
| 172443_x_at | Y73F8A | Y73F8A.9 | pqn-91 | + | — |
| 175102_at | C04H5 | C04H5.7 | Unknown | − | — |
| 177489_at | C05E7 | C05E7.2 | Unknown | − | — |
| 178259_at | DY3 | F36A2.3 | Malate dehydrogenase domain | − | — |
| 180540_at | C42D4 | C42D4.3 | Fibronectin domain | − | — |
| 182415_s_at | F48E3 | F48E3.4 | Peptidase S1 and S6 domain | − | — |
| 182599_s_at | W02G9 | W02G9.4 | CUB domain | + | — |
| 182628_s_at | T21F4 | T21F4.1 | Arginase domain | + | Arginase 1 |
| 183115_at | C34H4 | C34H4.2 | Unknown | − | — |
| 183187_at | K10D11 | K10D11.1 | Unknown | − | — |
| 184393_at | C33H5 | C33H5.13 | Unknown | − | — |
| 184943_s_at | T02G5 | T02G5.11 | Nanos RNA binding domain | − | — |
| 185954_at | Y54G11A | Y54G11A.7 | Tetratricopeptide repeat domain | + | — |
| 185964_at | Y37D8A | Y37D8A.19 | Unknown | − | — |
| 187358_at | F07F6 | F07F6.5 | Unknown | − | — |
| 189178_at | F44G3 | F44G3.2 | ATP: guanidophosphotransferase | + | — |
| | | | domain | | |
| 189953_at | T11F9 | T11F9.3 | nas-20 | + | — |
| 191251_at | C33A12 | C33A12.6 | UDP-glucuronosyl and UDP-glucosyl | + | UDP- |
| | | | transferase domain | | glucuronosyl- |
| | | | | | transferase |
| | | | | | 2A1 |
| | | | | | precursor |
| 191470_s_at | C31C9 | C31C9.1a, C31C9.1b | tag-10 | + | mucin-2 |
| 192956_at | F11G11 | F11G11.3 | GST-6 | + | — |
| | | 20 | | 9 | 3 |
| [symbol] | | | | | |
| 192194_s_at | W03G1 | W03G1.7 | asm-3 | + | SMPD1 |
| 174393_at | H40L08 | H40L08.2 | pseudogene predicted | − | — |
| 179360_s_at | T16G1 | T16G1.7 | Unknown | − | — |
| 179879_at | Y51H4A | Y51H4A.5 | Lipase domain | − | — |
| | | 4 | | 1 | 1 |
| [symbol] | | | | | |
| 173051_s_at | F22A3 | F22A3.6a | Destabilase domain | − | — |
| 176474_at | Y54F10AM | Y54F10AM.6 | Unknown | − | — |
| 176864_at | Y5H2A | Y5H2A.1 | Unknown | − | — |
| 177375_at | M60 | M60.2 | Unknown | + | — |
| 177920_at | D1086 | D1086.3 | Unknown | − | — |
| 178149_s_at | T07C4 | T07C4.4 | spp-1 | − | — |
| 178409_s_at | F58B3 | F58B3.3 | Lys-6 | − | — |
| 180913_at | F32A5 | F32A5.5a, F32A5.5b | aqp-1 | + | — |
| 181156_at | T08A9 | T08A9.8 | spp-4 | + | — |
| 182330_s_at | Y37A1A | Y37A1A.2 | Unknown | + | — |
| 183392_at | C02B8 | K07E3.1 | Similar to hemogglutinin domain | + | Dentin |
| | | | | | sialophosphoprotein |
| | | | | | precursor |
| 184079_at | R09B5 | R09B5.4 | Iron transporter domain | + | — |
| 184662_at | Y46G5A | Y46G5A.29 | ShTK domain | + | Notch 2 |
| 187247_at | F59B10 | F59B10.5 | Unknown | − | — |
| 187996_s_at | F28H7 | F28H7.3 | Lipase domain | − | — |
| 188037_at | ZK377 | ZK377.1 | wrt-6 | + | Indian hedgehog |
| | | | | | protein precursor |
| 188078_at | F46B6 | F46B6.8 | Triglyceride lipase domain | + | — |
| 188802_at | F54D5 | F54D5.8 | dnj-13 | + | — |
| 189732_at | F49E12 | F49E12.9 | Sterol desaturase domain | + | — |
| 190987_at | R13H4 | R13H4.3 | Histidine acid phosphatase domain | + | — |
| 192144_s_at | ZK455 | ZK455.4 | asm-2 | + | SMPD1 |
| 193836_s_at | W04E12 | W04E12.8 | C-type lectin domain | + | mannose receptor, |
| | | | | | C type 2 |
| | | 22 | | 14 | 5 |

| Affy ID | TMDs | Transporters | Metal/Zinc finger | Nucleic acid | Known |
|---|---|---|---|---|---|
| >2(all inc) | | | | | |
| 176851_at | | | | | |
| 180150_at | | | | | |
| 184035_at | | | | | |
| 189419_at | 1_C | | | | |
| 176681_at | | | | | |
| 179098_at | | | | | |
| 183117_at | | | | | |
| 183379_at | | | | | |
| 183676_s_at | | | | | |
| 185399_at | | ptn transport in | | | |
| | | yeast | | | |
| 187962_at | 1_N | | | | |
| 190693_at | 2 | | | | |
| 191418_at | 1_C | UDP transferase | | | |
| 180973_at | | | heme | | |
| | 4 | 2 | 1 | | |
| 1.6-2(all inc) | | | | | |
| 173836_at | | | hemopexin repeat | | toh-1: tollish |
| 182573_at | | | | | |
| 184313_s_at | | | | | |
| 184399_at | | | | | |
| 185343_at | | | | | |
| 185425_at | | | | | |
| 187662_at | 1_N | | | | |
| 188028_at | | | | | grd-6: hedgehog-like family |
| 192059_at | 10 | sugar transporter | | | |
| | | family | | | |
| 194067_at | | | | | |
| | 2 | 1 | 1 | | 2 |
| >2 All Dec. | | | | | |
| 175239_at | | | | | |
| 186519_at | | | | | |
| 183724_at | 1_N | | | | |
| 171941_s_at | | | | | |
| 172400_x_at | | | | | |
| 172971_s_at | 1_M | | | + | |
| 176395_at | | | | | |
| 177613_at | 1_N | | | | |
| 177812_at | 12 | glpT transporter | | | |
| 178087_s_at | | | | | lys-5 |
| 180616_at | | | | | |
| 180706_at | 1_N | | | | |
| 181502_s_at | 8 | | | | |
| 181520_at | 11 | | | | |
| 183273_at | 1_N | | | | |
| 185242_at | | | | | |
| 188495_at | 9 | phosphate | | | |
| | | transport | | | |
| 189595_s_at | 1_N | | | | |
| 190958_s_at | | | | | |
| 191882_at | 1_C | | | | |
| 193588_s_at | | | | | |
| | 11 | 2 | | 1 | 1 |
| 1.6-2(alt dec) | | | | | |
| 180880_at | 1_N | | | | |
| 181819_s_at | | | | | |
| 181902_at | | | | | |
| 187382_at | 1_N | | | | |
| 187846_at | | | | | |
| 189975_at | | | | | |
| 193034_at | | | | | |
| | 2 | | | | |
| >2 (De4/In500) | | | | | |
| 180562_at | | | | | |
| 171932_x_at | 1_N | | | | |
| 172184_x_at | | | | | |
| 178297_at | | | zinc finger | | |
| 178843_at | | | | | |
| 179424_at | | | | | |
| 182970_at | | | | | |
| 183527_s_at | | | | | |
| 187964_at | | | | | |
| 188441_at | | | | | |
| 192509_at | | | | | |
| | 1 | | 1 | | |
| >2 (in 4/De500) | | | | | |
| 172134_x_at | | lipid transport ptn | | | vit-4 |
| 173411_s_at | | lipid transport ptn | | | vit-6 |
| 175993_at | | | | | |
| 188947_at | 1_N | | | | |
| 189227_at | | | | | |
| 189660_at | 1_N | | Calcium bind | | |
| 190619_at | | | | | |
| 180752_s_at | | Solute carrier, Zinc | | | |
| | | transporter | | | |
| 194239_x_at | | | | | vit-3 |
| | 2 | 3 | 1 | | 3 |
| 1.6 to 2 | | | | | |
| 177428_at | 3 | Ctr family | | | |
| | 1 | 1 | | | |
| >2(NC4/In500) | | | | | |
| 177816_at | 4 | | | | |
| 178017_s_at | | | | | |
| 179933_at | | | | | |
| 180315_at | 4 | | | | |
| 180727_at | | | | | |
| 181099_at | 1_C | | | | |
| 181946_at | | | | | |
| 183020_at | | | | | |
| 183526_at | | | | | |
| 183665_s_at | | | | | |
| 183850_at | | | | | |
| 184116_s_at | | | | | |
| 184352_at | | | | | |
| 184624_s_at | | | | | |
| 184707_s_at | | | | | |
| 185145_at | | | | | |
| 188106_at | | | Iron-sulphur bind | | |
| 188465_s_at | 1_N | | | | |
| 188987_at | | | | | |
| 189971_at | | | | | |
| 190139_s_at | 1_N | | | | |
| 190830_at | | | | | |
| 191502_at | 1_C | | | | |
| 191568_at | | | | | |
| 192076_at | 4 | MIP family | | | |
| 193007_s_at | 11 | ABC transporter | | | pgp-1 |
| 194063_at | | | | | |
| | 8 | 2 | 1 | | 1 |
| 1.6-2(NC4/In500) | | | | | |
| 172069_x_at | | | | | cpr-3 - (Cysteine |
| | | | | | PRotease related) |
| 172503_x_at | | | Iron-sulphur bind | | |
| 172769_x_at | | | | | |
| 173090_s_at | | | | | |
| 175170_s_at | | | | | |
| 175870_s_at | | | | | |
| 176007_at | 5 | | | | |
| 176026_at | | | | | |
| 176360_at | | | Zinc finger | | |
| 176756_s_at | | | | | |
| 176826_at | | | | | |
| 177059_at | | | | | |
| 177187_at | | | | | |
| 177219_at | 5 | | | | |
| 177693_at | 1_N | | | | |
| 177827_at | | | | | |
| 178128_at | | | | | |
| 178403_at | | | | | |
| 179682_at | | | | | |
| 179989_at | | | | | |
| 180379_s_at | | | | | |
| 180410_at | | | | | |
| 180642_at | | | Zinc finger | + | |
| 180810_at | 7 | | | | |
| 181459_at | 4 | | | | |
| 182106_at | 1_N | | | | |
| 182276_at | | | | | |
| 182279_at | | | | | |
| 182712_at | | | | | |
| 182790_at | | | | | |
| 183702_at | | | | | |
| 183867_at | | | | | |
| 186413_at | 1_C | | | | |
| 186528_s_at | | | Zinc finger | | |
| 186799_at | | | | | |
| 186832_at | | | | | |
| 187548_s_at | | | | | |
| 188648_at | | | | | |
| 189179_at | | | | | hil-1 - (Histone H1 Like) |
| 189325_at | | | | | |
| 189473_at | 15 | abt-4; ABC | | | |
| | | transporter | | | |
| 190156_s_at | | | | | |
| 190301_s_at | 10 | ammonium | | | |
| | | transporter | | | |
| 190413_at | | | | | |
| 190899_at | | | | | |
| 190959_s_at | | | | | |
| 191298_s_at | 1_N | | | | |
| 191336_at | 7 | | | | |
| 191406_at | | multicopper | | | |
| | | oxidases | | | |
| 191541_at | 1_N | | | | |
| 191970_at | | | Zinc finger | | odd-2 - (Drosophilla ODD- |
| | | | | | skipped-like) |
| 192249_at | | | | | |
| 192559_s_at | | | | | |
| 192596_s_at | | | | | |
| 192610_at | | | | | nas-12 - (Nematode |
| | | | | | AStacin protease) |
| 192628_at | 4_C | | | | glc-1 - (Glutamate-gated |
| | | | | | ChLoride channel) |
| 193165_at | 12 | ammonium | | | rhr-1 - (RH (Rhesus) |
| | | transporter RHBG | | | antigen Retated) |
| 193926_at | | | Zinc finger | | |
| | 14 | 4 | 6 | 1 | 6 |
| >2(NC4/De500) | | | | | |
| 176230_at | | | | | |
| 177532_at | 1_M | | | | |
| 179226_at | | | | | |
| 180997_at | | | | | |
| 183455_at | 1_N | | | | |
| 189366_at | | | | | |
| 190080_at | | | | | |
| 190962_s_at | | | | | |
| 193584_s_at | | | | | cyn-2 |
| | 2 | | | | 1 |
| 1.6-2(NG4/De500) | | | | | |
| 172904_x_at | | | | | |
| 174273_at | | | | | |
| 175898_at | | | | | |
| 176419_at | | | | | |
| 176461_s_at | | | | | |
| 177065_at | | lipid transport ptn | | | vit-1 - (VITellogenin |
| | | | | | structural genes (yolk |
| | | | | | protein genes)) |
| 177729_at | | | | | |
| 178189_at | | | | | |
| 178243_s_at | | | | | |
| 178439_s_at | | | | | |
| 178759_at | | | | | |
| 178929_at | 1_C | | | | |
| 179199_at | | | | | |
| 180462_at | 1_N | | | | |
| 181331_s_at | | | | | |
| 181833_at | 2_C | | | | |
| 182078_at | 4 | | | | |
| 182498_at | | | | | |
| 183232_s_at | 3 | | | | |
| 183713_at | 2 | | | | |
| 184740_at | 2 | | | | |
| 184839_at | 6 | | | | |
| 184864_at | 1_N | | | | |
| 187612_s_at | | | | | |
| 188128_at | | | | | |
| 188849_at | | | | | |
| 188917_s_at | | | zinc finger | | |
| 189208_at | | | | | |
| 189921_at | | | | | |
| 190094_s_at | | | | | |
| 190585_at | | | | | |
| 191303_at | | | | | |
| 192175_at | 1_C | | | | |
| 192786_s_at | | | | | |
| 192819_at | | | zinc finger | | |
| 193127_s_at | | | | | dao-3 - (Dauer or Aging |
| | | | | | adult Overexpression) |
| 193885_at | 1_C | | Calcium bind | | |
| 194086_at | | | | | |
| | 11 | 1 | 3 | | 2 |
| >2( [symbol] n4/NC600) | | | | | |
| 174713_at | | | | | |
| 175810_at | | | zinc finger | | pos-1 |
| 177544_at | 2 | | | | |
| 179900_s_at | | | | | |
| 185275_at | 1_N | | | | cdr-5 |
| 186182_s_at | 4 | | | | |
| 186232_s_at | | | zinc finger | | |
| 189671_at | 1_N | | | | |
| 173316_s_at | | | | | |
| 174283_s_at | | | | | |
| 174964_at | | | | | |
| 175010_s_at | 1_N | | | | |
| 175377_at | | | | | |
| 176004_at | | | zinc finger | | |
| 177253_at | | | | | |
| 177389_at | 2 | | | | |
| 177820_at | | | | | |
| 178775_at | | | | + | |
| 179525_at | 3 | | | | |
| 179805_s_at | | | | | |
| 180173_at | | | | | |
| 180306_at | 1_N | trypsin inhibitor like | | | |
| | | Cys rich domain | | | |
| 180361_at | | | | | |
| 180676_at | | | | | |
| 182492_at | | | | | |
| 183721_at | | | | | |
| 185762_at | | | | | |
| 186231_at | | | | | |
| 192345_s_at | | | | | |
| | 8 | 1 | 3 | 1 | 2 |
| [symbol] | | | | | |
| 172443_x_at | | | | | |
| 175102_at | 1_N | GABA receptor | | | |
| 177489_at | | | | | |
| 178259_at | | | | | |
| 180540_at | | | | + | |
| 182415_s_at | | | | | |
| 182599_s_at | | | | | |
| 182628_s_at | | | | | |
| 183115_at | | | | | |
| 183187_at | | | | | |
| 184393_at | | | | + | |
| 184943_s_at | | | | + | |
| 185954_at | | | | | |
| 185964_at | | | | | |
| 187358_at | | | zinc finger | | |
| 189178_at | | | | | |
| 189953_at | | | Hemopexin repeat | | nas-20 - (Nematode |
| | | | | | AStacin protease) |
| 191251_at | 1_C | | | | |
| 191470_s_at | | | | | |
| 192956_at | | | | | |
| | 2 | 1 | 2 | 3 | 1 |
| [symbol] | | | | | |
| 192194_s_at | | | | | |
| 174393_at | | | | | |
| 179360_s_at | | | | | |
| 179879_at | 2 | | | | |
| | 1 | | | | |
| [symbol] | | | | | |
| 173051_s_at | | | | | |
| 176474_at | | | | + | |
| 176864_at | | | | | |
| 177375_at | | | | | |
| 177920_at | | | | | |
| 178149_s_at | | | | | spp-1: saposin-like protein |
| | | | | | family |
| 178409_s_at | | | | | |
| 180913_at | 6 | MIP family | | | aqp-1 - (AQuaPorin or |
| | | | | | aquaglyceroporin related) |
| 181156_at | 1_N | | | | spp-4 - (SaPosin-like |
| | | | | | Protein family) |
| 182330_s_at | 12 | H+ transporting 2- | | | |
| | | sector ATPase | | | |
| 183392_at | 4_N | | | | |
| 184079_at | 9 | solute carrier | | | |
| | | protein | | | |
| 184662_at | | | Calcium bind | | |
| 187247_at | | | | | |
| 187996_s_at | 1_N | | | | |
| 188037_at | | | | | wrt-6 - (WaRThog |
| | | | | | (hedgehog-like family)) |
| 188078_at | | | | | |
| 188802_at | | | | | |
| 189732_at | 2_N | | | | |
| 190987_at | | | | | |
| 192144_s_at | | | | | |
| 193836_s_at | | | | | |
| | 7 | 3 | 1 | 1 | 4 |

For Table 3: Unrelated genes repeated more than 4 times were removed from the table.

| Affy ID | Wormbase | Wormbase | Worm Profile | humans |
|---|---|---|---|---|
| >2(all inc) | | | | |
| 176851_at | Y40B10A | Y40B10A.6 | O-methyl transferase domain | + |
| 180150_at | F54E2 | F54E2.1 | Unknown | − |
| 189419_at | F15B9 | F15B9.6 | Phospholipase domain | − |
| 176681_at | F35B3 | F35B3.4 | Fibronectin domain | − |
| 179098_at | T19C9 | T19C9.8 | Unknown | − |
| 183117_at | ZK742 | ZK742.3 | NADH: Flavin oxireductase domain | − |
| 183379_at | K01D12 | K01D12.9 | Unknown | − |
| 183676_s_at | F08F8 | F08F8.5 | Unknown | + |
| 185399_at | Y75B8A | Y75B8A.28 | Unknown | − |
| 187962_at | K02E2 | K02E2.4 | Ins-35 | − |
| | | 10 | | 2 |
| 1.6-2(all inc) | | | | |
| 173836_at | T24A11 | T24A11.3 | Toh-1 | + |
| 184313_s_at | K11H12 | K11H12.4 | Unknown | − |
| 184399_at | Y26D4A | Y26D4A.10, Y26D4A.11 | Unknown | − |
| 185343_at | T28C12 | T28C12.6 | Unknown | − |
| 187662_at | F32H5 | F32H5.3a, F32H5.3b | Unknown | − |
| 188028_at | F07C3 | T18H9.1 | grd-6 | + |
| 192059_at | Y51A2D | Y51A2D.4 | MFS domain | + |
| | | 7 | | 3 |
| >2 All Dec | | | | |
| 175239_at | F15E11 | F15E11.15 | Unknown | − |
| 186519_at | F15E11 | F15E11.12, F15E11.15 | Unknown | − |
| 183724_at | Y11D7A | Y11D7A.5 | Unknown | − |
| 171941_s_at | F44E5 | F44E5.5 | Hsp70 domain | + |
| 172400_x_at | F22A3 | F22A3.6a, F22A3.6b | Destabilase domain | − |
| 172971_s_at | Y54G2A | Y54G2A.11a, Y54G2A.11b | Myb domain | + |
| 176395_at | Y71G12B | Y71G12B.18 | Unknown | − |
| 177613_at | F57G8 | F57G8.7 | Unknown | − |
| 177812_at | F10C2 | F10C2.7 | MFS domain | + |
| 178087_s_at | F58B3 | F58B3.2 | Lys-5 | − |
| 180616_at | T22B7 | T22B7.3 | Amidinotransferase domain | − |
| 180706_at | K06H6 | K06H6.2 | Unknown | − |
| 181502_s_at | W07A12 | W07A12.6 | Acyltransferase domain | − |
| 181520_at | W07A12 | W07A12.7 | Acyltransferase domain | − |
| 183273_at | C14C6 | C14C6.3 | Glycosyl transferase domain | − |
| 185242_at | Y105C5B | Y105C5B.7 | Calcineurin like phosphoesterase | + |
| | | | domain | |
| 188495_at | F09G2 | F09G2.3 | Phosphate transporter domain | + |
| 189595_s_at | K10C2 | K10C2.3 | Aspartyl protease domain | + |
| 190958_s_at | F44E5 | F44E5.4 | Hsp70 domain | + |
| 193588_s_at | F28D1 | F28D1.5 | thn-2 | − |
| | | 20 | | 7 |
| 1.6-2(all dec) | | | | |
| 180880_at | K06H6 | K06H6.1 | Unknown | − |
| 181819_s_at | Y37D8A | Y37D8A.4 | SH2 domain | + |
| 181902_at | Y43D4A | Y43D4A.5 | Unknown | + |
| 187382_at | F55C10 | F55C10.4 | Unknown | − |
| 187846_at | W03F11 | W03F11.5a, W03F11.5b | Unknown | − |
| 193034_at | F56H6 | F56H6.5 | GDP-mannose 4,6-dehydratase domain | + |
| | | 6 | | 3 |
| >2 (De4/In500) | | | | |
| 171932_x_at | F09C8 | F09C8.1 | Phospholipase domain | + |
| 172184_x_at | Y46C8AL | Y46C8AL.2 | Unknown | + |
| 178843_at | F08G5 | F08G5.6 | Unknown | − |
| 179424_at | F27C8 | F27C8.4 | spp-18 | − |
| 182970_at | K10D11 | F55G11.4 | Unknown | − |
| 187964_at | F54F3 | F54F3.3 | Lipase domain | + |
| 188441_at | F21F8 | F21F8.4 | Aspartyl protease domain | + |
| | | 7 | | 4 |
| >2 (In 4/De500) | | | | |
| 189227_at | W07B8 | W07B8.1 | Cystein protease domain | + |
| 189660_at | F59D6 | F59D6.3 | Aspartyl protease domain | + |
| 190619_at | C15C8 | C15C8.3 | Aspartyl protease domain | + |
| 180752_s_at | D1054 | D1054.10 | Unknown | − |
| | | 4 | | 3 |
| 1.6 to 2 | | | | |
| 177428_at | F58G6 | F58G6.3, F58G6.7 | Ctr domain | + |
| | | 1 | | 1 |
| >2(NC4/In500) | | | | |
| 177816_at | F49H6 | F49H6.3 | Unknown | − |
| 179933_at | F39E9 | F39E9.1 | Unknown | − |
| 180315_at | F44G3 | F44G3.10 | Unknown | − |
| 183850_at | Y48E1B | Y48E1B.8 | Unknown | − |
| 184116_s_at | T05E12 | T05E12.6 | Unknown | − |
| 184352_at | C17H12 | C17H12.6 | Unknown | − |
| 184624_s_at | C25H3 | C25H3.10, C25H3.10a, C25H3.10b | F-box domain | − |
| 184707_s_at | C32H11 | C32H11.10 | dod-21 | − |
| 185145_at | Y46D2A | Y46D2A.2 | Unknown | − |
| 188106_at | T01C3 | T01C3.4 | Lipase domain | − |
| 188465_s_at | R09D1 | R09D1.8 | glycosyl hydrolases domain | + |
| 188987_at | F20G2 | F20G2.1 | Short chain dehydrogenase domain | + |
| 189971_at | F01G10 | F01G10.3 | ech-9 | + |
| 190139_s_at | E03H4 | E03H4.10 | CUB domain | + |
| 190830_at | T21C9 | T21C9.8 | Transthyretin like domain | − |
| 192076_at | K02G10 | K02G10.7a, K02G10.7b | aqp-8 | + |
| 193007_s_at | K08E7 | K08E7.9 | pgp-1 | + |
| | | 17 | | 6 |
| 1.6-2(NC4/In500) | | | | |
| 172069_x_at | T10H4 | T10H4.12 | cpr-3 | + |
| 172503_x_at | W08E12 | W08E12.3 | 2Fe-2S-ferredoxin domain | + |
| 172769_x_at | F44C8 | F44C8.1 | cyp-33C4 | + |
| 175170_s_at | ZK6 | ZK6.10 | dod-19 | − |
| 175870_s_at | Y34F4 | Y34F4.4 | Unknown | − |
| 176007_at | Y50D4A | Y50D4A.1 | Ribosome protein S2 domain | + |
| 176026_at | C06E1 | C06E1.3 | Unknown | + |
| 176360_at | R13A5 | R13A5.10 | Cytidine deaminase domain | + |
| 176756_s_at | Y65B4A | F56A6.1a, F56A6.1b | Piwi domain | + |
| 176826_at | Y40B10A | Y40B10A.2 | O-methyl transferase domain | + |
| 177219_at | M28 | M28.8 | Glutamine amidotransferase domain | − |
| 177693_at | R03G8 | R03G8.3 | Unknown | − |
| 177827_at | T19C4 | T19C4.5 | Unknown | − |
| 178403_at | F10A3 | F10A3.2 | FTH domain | − |
| 179682_at | C31H5 | C31H5.6 | Acyl-CoA thioester hyrolase domain | + |
| 179989_at | F36G9 | F36G9.14 | FTH domain | − |
| 180379_s_at | C17F4 | C17F4.7 | Unknown | − |
| 180642_at | F53A9 | F53A9.2 | Peptidase M domain | + |
| 180810_at | R10D12 | R10D12.9 | MtN3/saliva family domain | + |
| 181459_at | Y102A5C | F49H6.13 | Unknown | − |
| 182106_at | C34H4 | C34H4.1 | Unknown | − |
| 182276_at | T10B10 | T10B10.4, T10B10.4a | Unknown | − |
| 182279_at | Y47H9C | Y47H9C.1 | Unknown | − |
| 183702_at | K05B2 | K05B2.4 | Acyl-CoA thioester hyrolase domain | + |
| 183867_at | C45E5 | C45E5.4 | Unknown | − |
| 186413_at | H20E11 | H20E11.1a, H20E11.1b | Unknown | − |
| 186528_s_at | F46E10 | F46E10.11 | Unknown | + |
| 186799_at | Y105C5B | Y105C5B.15 | Calcineurin like phosphoesterase | + |
| | | | domain | |
| 186832_at | F27E5 | F27E5.1 | Acid ceramidase | + |
| 187548_s_at | C32D5 | C32D5.6 | Unknown | + |
| 188648_at | C45B2 | C45B2.5 | Glutamine synthase domain | + |
| 189179_at | C30G7 | C30G7.1 | hil-1 | + |
| 189325_at | F02D8 | F02D8.4 | Zinc carboxylpeptidase domain | + |
| 189473_at | Y39D8C | Y39D8C.1 | abt-4 | + |
| 190156_s_at | C48B4 | C48B4.1 | Acyl-CoA dehydrogenase domain | + |
| 190301_s_at | C05E11 | C05E11.5 | amt-4 | + |
| 190413_at | F25G6 | F25G6.6 | nrs-2 | + |
| 191298_s_at | F23B2 | F23B2.12 | pcp-2 | + |
| 191336_at | K06C4 | K06C4.8 | rhodopsin domain | + |
| 191406_at | F21D5 | F21D5.3 | Multicopper oxidase domain | − |
| 191970_at | C34H3 | C34H3.2 | odd-2 | + |
| 192249_at | F22A3 | F22A3.1 | SAM/pointed domain | + |
| 192596_s_at | T03F7 | T03F7.7a, T03F7.7b | CRAL/TRIO domain | + |
| 192610_at | C24F3 | C24F3.3 | nas-12 | + |
| 192628_at | F11A5 | F11A5.10 | glc-1 | + |
| 193165_at | F08F3 | F08F3.3 | rhr-1 | + |
| 193926_at | Y48A6B | Y48A6B.7 | Cytidine deaminase domain | + |
| | | 47 | | 32 |
| >2(NC4/De500) | | | | |
| 176230_at | Y71H2AM | Y71H2AM.16 | Histidine acid phosphatase domain | + |
| 177532_at | F22B5 | F22B5.4 | Unknown | − |
| 179226_at | C06B3 | C06B3.7 | Unknown | − |
| 180997_at | C34D4 | C34D4.3 | MSP domain | + |
| 183455_at | F26B1 | F26B1.1 | Unknown | + |
| 189366_at | K07C6 | K07C6.4 | cyp-35B1 | + |
| 190080_at | F26C11 | F26C11.1 | Histidine acid phosphatase domain | + |
| 193584_s_at | ZK520 | ZK520.5 | cyn-2 | + |
| | | 8 | | 6 |
| 1.6-2(NC4/De500) | | | | |
| 172904_x_at | K05F1 | K05F1.7 | msp-63 | + |
| 174273_at | F01G10 | F01G10.9 | Unknown | + |
| 175898_at | ZK105 | ZK105.1 | Unknown | − |
| 176419_at | Y71G12B | Y71G12B.17 | Phosphatidylinositol transfer protein | + |
| | | | domain | |
| 176461_s_at | Y59H11AM | Y59H11AM.3 | Rhodanese-like domain | + |
| 177729_at | E01G4 | E01G4.6 | Phospholipase domain | + |
| 178189_at | F28C6 | F28C6.5 | Unknown | − |
| 178243_s_at | F32B6 | F32B6.4 | Unknown | − |
| 178439_s_at | F17C8 | F17C8.7 | Unknown | − |
| 178759_at | F59C6 | F59C6.6 | nlp-4 | − |
| 178929_at | T03F7 | F47G9.3 | Zona pellucida-like domain | − |
| 179199_at | T09F5 | T09F5.1 | Galactosyltransferase domain | + |
| 180462_at | C07E3 | C07E3.10 | Unknown | − |
| 181331_s_at | K09C6 | K09C6.8 | Unknown | + |
| 181833_at | K01A2 | K01A2.4 | Unknown | + |
| 182078_at | T10E9 | T10E9.8 | Unknown | − |
| 182498_at | H25K10 | H25K10.1 | Calcineurin like phosphoesterase | + |
| | | | domain | |
| 183232_s_at | K01A2 | K01A2.3 | Unknown | − |
| 183713_at | C24G7 | C24G7.2 | Amiloride-sensitive sodium channel | + |
| | | | domain | |
| 184740_at | C33F10 | C33F10.1 | Unknown | − |
| 184839_at | T06C10 | C55F2.1b | ALCARFT/IMPCHase bienzyme domain | + |
| 184864_at | C08F11 | C08F11.11 | Unknown | − |
| 187612_s_at | F47D12 | F47D12.7 | Kelch motif domain | + |
| 188128_at | C50B6 | C50B6.7 | Alpha-amylase domain | + |
| 188849_at | C56E6 | C56E6.2 | Ras domain | + |
| 188917_s_at | F26H11 | F26H11.2c, F26H11.2d | DDT domain | + |
| 189208_at | F38B6 | F38B6.4 | phosphoribosylglycinamide synthase | + |
| | | | domain | |
| 189921_at | C05C10 | C05C10.4 | Histidine acid phosphatase domain | + |
| | | M02D8.4a, M02D8.4b, M02 | | |
| 190094_s_at | M02D8 | D8.4c | Asparagine synthase domain | + |
| 190585_at | ZK970 | ZK970.7 | Unknown | − |
| 192175_at | C27D6 | C27D6.3 | Unknown | + |
| 192786_s_at | B0286 | B0286.3 | SAICAR synthase domain | + |
| 192819_at | K10C3 | Y67A6A.2 | nhr-62 | + |
| 193127_s_at | K07E3 | K07E3.3 | dao-3 | + |
| 193885_at | B0393 | B0393.5 | EGF-like domain | + |
| 194086_at | W01F3 | W01F3.3 | Trypsin Inhibitor domain | + |
| | | 36 | | 24 |
| >2(1n4/NC500) | | | | |
| 174713_at | F14F4 | F14F4.3a | mrp-5 | − |
| 175810_at | F52E1 | F52E1.1 | pos-1 | + |
| 177544_at | F58E6 | F58E6.7, F58E6.11 | Unknown | − |
| 179900_s_at | C44B7 | C44B7.5 | Cyt b-561/ferric reductase domain | − |
| 185275_at | C54D10 | K01D12.14 | cdr-5 | + |
| 186182_s_at | R02E12 | R02E12.6 | Unknown | + |
| 186232_s_at | C44B12 | C44B12.1 | Unknown | − |
| 189671_at | C50H11 | C50H11.15 | cyp-33C9 | + |
| 173316_s_at | Y62H9A | Y62H9A.6 | Unknown | − |
| | | | Phosphoribosylglycinamide synthase | |
| 174283_s_at | F38B6 | F38B6.4 | domain | + |
| 174964_at | K02B9 | K02B9.1 | Unknown | − |
| 175010_s_at | F57C2 | F57C2.4 | Unknown | − |
| 175377_at | ZK813 | ZK813.1 | Unknown | − |
| 176004_at | M02H5 | M02H5.4 | Zinc finger domain | + |
| 177253_at | R186 | R186.1 | Unknown | + |
| 177389_at | F58E6 | F58E6.8 | Unknown | − |
| 177820_at | ZC373 | ZC373.2 | Unknown | − |
| 178775_at | C01G6 | C01G6.3 | Unknown | − |
| 179525_at | F36H1 | F36H1.5 | Unknown | − |
| 179805_s_at | T04G9 | T04G9.7 | Unknown | − |
| 180173_at | Y62H9A | Y62H9A.4 | Unknown | − |
| 180306_at | C10G8 | C10G8.4 | Trypsin inhibitor domain | + |
| 180361_at | F42G2 | F42G2.2 | FTH domain | − |
| 180676_at | ZK813 | ZK813.1 | Chorion domain | + |
| 182492_at | C16C4 | C16C4.4 | MATH domain | − |
| 183721_at | F17E9 | F17E9.4 | Unknown | − |
| 185762_at | F47C10 | F47C10.2 | BTB domain | + |
| 186231_at | C44B12 | C44B12.1 | Unknown | − |
| 192345_s_at | F32H5 | F32H5.1 | Cystein protease domain | + |
| | | 29 | | 11 |
| (1.6-2(In4/NC500) | | | | |
| 172443_x_at | Y73F8A | Y73F8A.9 | pqn-91 | + |
| 175102_at | C04H5 | C04H5.7 | Unknown | − |
| 177489_at | C05E7 | C05E7.2 | Unknown | − |
| 178259_at | DY3 | F36A2.3 | Malate dehydrogenase domain | − |
| 180540_at | C42D4 | C42D4.3 | Fibronectin domain | − |
| 182415_s_at | F48E3 | F48E3.4 | Peptidase S1 and S6 domain | − |
| 182599_s_at | W02G9 | W02G9.4 | CUB domain | + |
| 182628_s_at | T21F4 | T21F4.1 | Arginase domain | + |
| 183115_at | C34H4 | C34H4.2 | Unknown | − |
| 183187_at | K10D11 | K10D11.1 | Unknown | − |
| 184393_at | C33H5 | C33H5.13 | Unknown | − |
| 184943_s_at | T02G5 | T02G5.11 | Nanos RNA binding domain | − |
| 185954_at | Y54G11A | Y54G11A.7 | Tetratricopeptide repeat domain | + |
| 185964_at | Y37D8A | Y37D8A.19 | Unknown | − |
| 187358_at | F07F6 | F07F6.5 | Unknown | − |
| 189178_at | F44G3 | F44G3.2 | ATP: guanidophosphotransferase | |
| | | | domain | + |
| 189953_at | T11F9 | T11F9.3 | nas-20 | + |
| 191470_s_at | C31C9 | C31C9.1a, C31C9.1b | tag.10 | + |
| | | 18 | | 7 |
| >2(Do4/NC500) | | | | |
| 192194_s_at | W03G1 | W03G1.7 | asm-3 | + |
| 174393_at | H40L08 | H40L08.2 | pseudogene predicted | − |
| 179360_s_at | T16G1 | T16G1.7 | Unknown | − |
| 179879_at | Y51H4A | Y51H4A.5 | Lipase domain | − |
| | | 4 | | 1 |
| (1.6-2(Do4/NC500) | | | | |
| 173051_s_at | F22A3 | F22A3.6a | Destabilase domain | − |
| 176474_at | Y54F10AM | Y54F10AM.6 | Unknown | − |
| 176864_at | Y5H2A | Y5H2A.1 | Unknown | − |
| 177375_at | M60 | M60.2 | Unknown | + |
| 177920_at | D1086 | D1086.3 | Unknown | − |
| 178149_s_at | T07C4 | T07C4.4 | spp-1 | − |
| 178409_s_at | F58B3 | F58B3.3 | Lys-6 | − |
| 180913_at | F32A5 | F32A5.5a, F32A5.5b | aqp-1 | + |
| 181156_at | T08A9 | T08A9.8 | spp-4 | + |
| 182330_s_at | Y37A1A | Y37A1A.2 | Unknown | + |
| 183392_at | C02B8 | K07E3.1 | Similar to hemogglutinin domain | + |
| 184079_at | R09B5 | R09B5.4 | Iron transporter domain | + |
| 187247_at | F59B10 | F59B10.5 | Unknown | − |
| 187996_s_at | F28H7 | F28H7.3 | Lipase domain | − |
| 188037_at | ZK377 | ZK377.1 | wrt-6 | + |
| 188078_at | F46B6 | F46B6.8 | Triglyceride lipase domain | + |
| 188802_at | F54D5 | F54D5.8 | dnj-13 | + |
| 189732_at | F49E12 | F49E12.9 | Sterol desaturase domain | + |
| 190987_at | R13H4 | R13H4.3 | Histidine acid phosphatase domain | + |
| 192144_s_at | ZK455 | ZK455.4 | asm-2 | + |
| | | 20 | | 12 |

| | | | | Metal/ | | |
|---|---|---|---|---|---|---|
| | | | | Zinc | | |
| Affy ID | Knownhuman | TMDs | Transporters | finger | Nucleic acid | Known |
| >2(all inc) | | | | | | |
| 176851_at | — | | | | | |
| 180150_at | — | | | | | |
| 189419_at | — | 1_C | | | | |
| 176681_at | — | | | | | |
| 179098_at | — | | | | | |
| 183117_at | — | | | | | |
| 183379_at | — | | | | | |
| 183676_s_at | — | | | | | |
| 185399_at | — | | ptn transport in | | | |
| | | | yeast | | | |
| 187962_at | — | 1_N | | | | |
| | | 2 | 1 | | | |
| 1.6-2(all inc) | | | | | | |
| 173836_at | — | | | hemopexin | | toh-1: tollish |
| | | | | repeat | | |
| 184313_s_at | — | | | | | |
| 184399_at | — | | | | | |
| 185343_at | — | | | | | |
| 187662_at | — | 1_N | | | | |
| 188028_at | mucin-2 | | | | | grd-6: hedgehog-like family |
| 192059_at | — | 10 | sugar transporter | | | |
| | | | family | | | |
| | 1 | 2 | 1 | 1 | | 2 |
| >2 All Dec | | | | | | |
| 175239_at | — | | | | | |
| 186519_at | — | | | | | |
| 183724_at | — | 1_N | | | | |
| 171941_s_at | — | | | | | |
| 172400_x_at | — | | | | | |
| 172971_s_at | — | 1_M | | | + | |
| 176395_at | — | | | | | |
| 177613_at | — | 1_N | | | | |
| 177812_at | NA/PI-4 | 12 | glpT transporter | | | |
| 178087_s_at | — | | | | | lys-5 |
| 180616_at | — | | | | | |
| 180706_at | — | 1_N | | | | |
| 181502_s_at | — | 8 | | | | |
| 181520_at | — | 11 | | | | |
| 183273_at | — | 1_N | | | | |
| 185242_at | — | | | | | |
| 188495_at | leukemia virus | 9 | phosphate | | | |
| | receptor 1 | | transport | | | |
| 189595_s_at | — | 1_N | | | | |
| 190958_s_at | HSP70A8 | | | | | |
| 193588_s_at | — | | | | | |
| | 3 | 10 | 2 | | 1 | 1 |
| 1.6-2(all dec) | | | | | | |
| 180880_at | — | 1_N | | | | |
| 181819_s_at | — | | | | | |
| 181902_at | MUC-1 | | | | | |
| 187382_at | — | 1_N | | | | |
| 187846_at | — | | | | | |
| 193034_at | — | | | | | |
| | 1 | 2 | | | | |
| >2 (De4/In500) | | | | | | |
| 171932_x_at | — | 1_N | | | | |
| 172184_x_at | — | | | | | |
| 178843_at | — | | | | | |
| 179424_at | — | | | | | |
| 182970_at | — | | | | | |
| 187964_at | — | | | | | |
| 188441_at | gastric cathepsin E | | | | | |
| | 1 | 1 | | 0 | | |
| >2 (In 4/De500) | | | | | | |
| 189227_at | Cathepsin B1 | | | | | |
| 189660_at | — | 1_N | | Calcium | | |
| | | | | bind | | |
| 190619_at | gastric cathepsin E | | | | | |
| 180752_s_at | | | Solute carrier, Zinc | | | |
| | | | transporter | | | |
| | 2 | 1 | 1 | 1 | | 0 |
| 1.6 to 2 | | | | | | |
| 177428_at | human Ctr1 | 3 | Ctr family | | | |
| | 1 | 1 | 1 | | | |
| >2(NC4/In500) | | | | | | |
| 177816_at | — | 4 | | | | |
| 179933_at | — | | | | | |
| 180315_at | — | 4 | | | | |
| 183850_at | — | | | | | |
| 184116_s_at | — | | | | | |
| 184352_at | — | | | | | |
| 184624_s_at | — | | | | | |
| 184707_s_at | — | | | | | |
| 185145_at | — | | | | | |
| 188106_at | — | | | Iron- | | |
| | | | | sulphur | | |
| | | | | bind | | |
| 188465_s_at | — | 1_N | | | | |
| 188987_at | — | | | | | |
| 189971_at | PBFE | | | | | |
| 190139_s_at | mannose receptor, | 1_N | | | | |
| | C type 2 | | | | | |
| 190830_at | — | | | | | |
| 192076_at | — | 4 | MIP family | | | |
| 193007_s_at | P-glycoprotein | 11 | ABC transporter | | | pgp-1 |
| | 3 | 6 | 2 | 1 | | 1 |
| 1.6-2(NC4/In500) | | | | | | |
| 172069_x_at | Cathepsin B1 | | | | | cpr-3 - (Cysteine PRotease |
| | | | | | | related) |
| 172503_x_at | — | | | Iron- | | |
| | | | | sulphur | | |
| | | | | bind | | |
| 172769_x_at | — | | | | | |
| 175170_s_at | — | | | | | |
| 175870_s_at | — | | | | | |
| 176007_at | — | 5 | | | | |
| 176026_at | — | | | | | |
| 176360_at | — | | | Zinc finger | | |
| 176756_s_at | — | | | | | |
| 176826_at | — | | | | | |
| 177219_at | — | 5 | | | | |
| 177693_at | — | 1_N | | | | |
| 177827_at | — | | | | | |
| 178403_at | — | | | | | |
| 179682_at | — | | | | | |
| 179989_at | — | | | | | |
| 180379_s_at | — | | | | | |
| 180642_at | — | | | Zinc finger | + | |
| 180810_at | — | 7 | | | | |
| 181459_at | — | 4 | | | | |
| 182106_at | — | 1_N | | | | |
| 182276_at | — | | | | | |
| 182279_at | — | | | | | |
| 183702_at | — | | | | | |
| 183867_at | — | | | | | |
| 186413_at | — | 1_C | | | | |
| 186528_s_at | — | | | Zinc finger | | |
| 186799_at | — | | | | | |
| 186832_at | Acid ceramidase | | | | | |
| | precursor | | | | | |
| 187548_s_at | — | | | | | |
| 188648_at | — | | | | | |
| 189179_at | — | | | | | hil-1 - (Histone H1 Like) |
| 189325_at | PCPB protein | | | | | |
| 189473_at | human ABCA3 | 15 | abt-4; ABC | | | |
| | | | transporter | | | |
| 190156_s_at | Acyl-coenzyme A | | | | | |
| | isoform b | | | | | |
| 190301_s_at | — | 10 | ammonium | | | |
| | | | transporter | | | |
| 190413_at | Asparagine | | | | | |
| | synthetase | | | | | |
| 191298_s_at | — | 1_N | | | | |
| 191336_at | — | 7 | | | | |
| 191406_at | — | | multicopper | | | |
| | | | oxidases | | | |
| 191970_at | — | | | Zinc finger | | odd-2 - (Drosophila ODD- |
| | | | | | | skipped-like) |
| 192249_at | — | | | | | |
| 192596_s_at | SEC14-like 2 | | | | | |
| 192610_at | — | | | | | nas-12 - (Nematode |
| | | | | | | AStacin protease) |
| 192628_at | — | 4_C | | | | glc-1 - (Glutamate-gated |
| | | | | | | ChLoride channel) |
| 193165_at | — | 12 | ammonium | | | rhr-1 - (RH (Rhesus) |
| | | | transporter RHBG | | | antigen Related) |
| 193926_at | — | | | Zinc finger | | |
| | 7 | 13 | 4 | 6 | 1 | 6 |
| >2(NC4/De500) | | | | | | |
| 176230_at | Prostatic acid | | | | | |
| | phosphatase | | | | | |
| 177532_at | — | 1_M | | | | |
| 179226_at | — | | | | | |
| 180997_at | — | | | | | |
| 183455_at | — | 1_N | | | | |
| 189366_at | cytochrome P450, | | | | | |
| | 2U | | | | | |
| 190080_at | — | | | | | |
| 193584_s_at | — | | | | cyn-2 | |
| | 2 | 2 | | | | 1 |
| 1.6-2(NC4/De500) | | | | | | |
| 172904_x_at | — | | | | | |
| 174273_at | — | | | | | |
| 175898_at | — | | | | | |
| 176419_at | phosphatidylinositol | | | | | |
| | transfer protein | | | | | |
| 176461_s_at | — | | | | | |
| 177729_at | — | | | | | |
| 178189_at | — | | | | | |
| 178243_s_at | — | | | | | |
| 178439_s_at | — | | | | | |
| 178759_at | — | | | | | |
| 178929_at | — | 1_C | | | | |
| 179199_at | — | | | | | |
| 180462_at | — | 1_N | | | | |
| 181331_s_at | — | | | | | |
| 181833_at | — | 2_C | | | | |
| 182078_at | — | 4 | | | | |
| 182498_at | — | | | | | |
| 183232_s_at | — | 3 | | | | |
| 183713_at | — | 2 | | | | |
| 184740_at | — | 2 | | | | |
| 184839_at | — | 6 | | | | |
| 184864_at | — | 1_N | | | | |
| 187612_s_at | — | | | | | |
| 188128_at | — | | | | | |
| 188849_at | — | | | | | |
| 188917_s_at | Fetal Alzheimer | | | zinc finger | | |
| | antigen isoform 1 | | | | | |
| 189208_at | phosphoribosyl- | | | | | |
| | glycinamide | | | | | |
| | fomyltransferase | | | | | |
| 189921_at | — | | | | | |
| 190094_s_at | Asparagine | | | | | |
| | synthetase | | | | | |
| 190585_at | — | | | | | |
| 192175_at | PARP1 protein | 1_C | | | | |
| 192786_s_at | — | | | | | |
| 192819_at | HNF4A | | | zinc finger | | |
| 193127_s_at | C-1− | | | | | dao-3 - (Dauer or Aging |
| | tetrahydrofolate | | | | | adult Overexpreasion) |
| | synthase | | | | | |
| 193885_at | Flbrillin 1 | 1_C | | Calcium | | |
| | | | | bind | | |
| 194086_at | — | | | | | |
| | 8 | 11 | 0 | 2 | | 1 |
| >2(1n4/NC500) | | | | | | |
| 174713_at | — | | | | | |
| 175810_at | — | | | zinc finger | | pos-1 |
| 177544_at | — | 2 | | | | |
| 179900_s_at | — | | | | | |
| 185275_at | — | 1_N | | | | cdr-5 |
| 186182_s_at | — | 4 | | | | |
| 186232_s_at | — | | | zinc finger | | |
| 189671_at | — | 1_N | | | | |
| 173316_s_at | — | | | | | |
| 174283_s_at | — | | | | | |
| 174964_at | — | | | | | |
| 175010_s_at | — | 1_N | | | | |
| 175377_at | — | | | | | |
| 176004_at | HNF4A | | | zinc finger | | |
| 177253_at | — | | | | | |
| 177389_at | — | 2 | | | | |
| 177820_at | — | | | | | |
| 178775_at | — | | | + | | |
| 179525_at | — | 3 | | | | |
| 179805_s_at | — | | | | | |
| 180173_at | — | | | | | |
| 180306_at | — | 1_N | trypsin inhibitor like | | | |
| | | | Cys rich domain | | | |
| 180361_at | — | | | | | |
| 180676_at | — | | | | | |
| 182492_at | — | | | | | |
| 183721_at | — | | | | | |
| 185762_at | — | | | | | |
| 186231_at | | | | | | |
| 192345_s_at | Cathepsin B1 | | | | | |
| | 2 | 8 | 1 | 3 | 1 | 2 |
| (1.6-2(In4/NC500) | | | | | | |
| 172443_x_at | — | | | | | |
| 175102_at | — | 1_N | GABA receptor | | | |
| 177489_at | — | | | | | |
| 178259_at | — | | | | | |
| 180540_at | — | | | | + | |
| 182415_s_at | — | | | | | |
| 182599_s_at | — | | | | | |
| 182628_s_at | Arginase 1 | | | | | |
| 183115_at | — | | | | | |
| 183187_at | — | | | | | |
| 184393_at | — | | | | + | |
| 184943_s_at | — | | | | + | |
| 185954_at | — | | | | | |
| 185964_at | — | | | | | |
| 187358_at | — | | | zinc finger | | |
| 189178_at | | | | | | |
| | — | | | | | |
| 189953_at | — | | | Hemopexin | | nas-20 - (Nematode |
| | | | | repeat | | AStacin protease) |
| 191470_s_at | mucin-2 | | | | | |
| | 2 | 1 | 1 | 2 | 3 | 0 |
| >2(Do4/NC500) | | | | | | |
| 192194_s_at | SMPD1 | | | | | |
| 174393_at | — | | | | | |
| 179360_s_at | — | | | | | |
| 179879_at | — | 2 | | | | |
| | 1 | 1 | | | | |
| (1.6-2(Do4/NC500) | | | | | | |
| 173051_s_at | — | | | | | |
| 176474_at | — | | | | + | |
| 176864_at | — | | | | | |
| 177375_at | — | | | | | |
| 177920_at | — | | | | | |
| 178149_s_at | — | | | | | spp-1: saposin-like protein |
| | | | | | | family |
| 178409_s_at | — | | | | | |
| 180913_at | — | 6 | MIP family | | | aqp-1 - (AQuaPorin or |
| | | | | | | aquaglyceroporin related) |
| 181156_at | — | 1_N | | | | spp-4 - (SaPosin-like |
| | | | | | | Protein family) |
| 182330_s_at | — | 12 | H+ transporting 2- | | | |
| | | | sector ATPase | | | |
| 183392_at | Dentin | 4_N | | | | |
| | sialophosphoprotein | | | | | |
| | precursor | | | | | |
| 184079_at | — | 9 | solute carrier | | | |
| | | | protein | | | |
| 187247_at | — | | | | | |
| 187996_s_at | — | 1_N | | | | |
| 188037_at | Indian hedgehog | | | | | wrt-6 - (WaRThog |
| | protein precursor | | | | | (hedgehog-like family)) |
| 188078_at | — | | | | | |
| 188802_at | — | | | | | |
| 189732_at | — | 2_N | | | | |
| 190987_at | — | | | | | |
| 192144_s_at | SMPD1 | | | | | |
| | 3 | 7 | 3 | 0 | 1 | 4 |

| Inc. Dec or no | Affy ID | | | | |
|---|---|---|---|---|---|
| change | Repeated genes | Wormbase | Wormbase | Worm Profile | humans |
| | 1 Collagen (27genes) | | | | |
| In4/De500 | 188335_at | T05A1 | T05A1.2 | COL-122 | + |
| In4/De500 | 188245_at | F15A2 | F15A2.1 | COL-184 | + |
| In4/De500 | 184144_at | C05D9 | R193.2 | Von Willebrand factor A domain | + |
| In4/De500 | 189911_s_at | F26F12 | F26F12.1 | COL-140 | + |
| De4/NC500 | 183010_s_at | C24B9 | C24B9.3a, C24B9.3b | Von Willebrand factor A domain | + |
| All De | 183666_at | C52D10 | C52D10.13 | COL-183 | + |
| All De | 188391_at | F59E12 | F59E12.12 | bli-2 | + |
| All De | 189482_s_at | C35B8 | C35B8.1 | COL175 | + |
| All De | 189864_s_at | F19C7 | F19C7.7 | COL-110 | + |
| All In | 190134_s_at | T10B10 | T10B10.1 | COL-41 | + |
| All In | 186383_at | T19D12 | T19D12.4a, T19D12.4b, | Von Willebrand factor A domain | + |
| | | | T19D12.4c, T19D12.5 | | |
| All In | 177024_at | C29E4 | C29E4.1 | COL-90 | + |
| In4/NC500 | 173647_s_at | C53B4 | C53B4.5 | COL-119 | + |
| In4/NC500 | 189561_at | W03G11 | W03G11.1 | COL-181 | + |
| In4/NC500 | 192163_at | ZK1193 | ZK1193.1a | COL-19 | + |
| In4/NC500 | 188406_s_at | F11H8 | F11H8.3 | COL-8 | + |
| In4/NC500 | 188456_at | T15B7 | T15B7.3 | COL-143 | + |
| In4/NC500 | 190530_s_at | F11G11 | F11G11.11 | COL-20 | + |
| In4/NC500 | 188747_at | Y54E10BL | Y54E10BL.2 | COL-48 | + |
| NC4/In500 | 188589_at | F33D11 | F33D11.3 | COL-54 | + |
| NC4/De500 | 173632_s_at | M18 | M18.1 | COL-129 | + |
| NC4/De500 | 173650_s_at | ZK265 | ZK265.2 | COL-63 | + |
| NC4/De500 | 174152_s_at | C09G5 | C09G5.6 | bli-1 | + |
| NC4/De500 | 174960_at | ZK1010 | ZK1010.7 | COL-97 | + |
| NC4/De500 | 186349_at | Y51H7C | Y51H7C.1 | Collagen domain | + |
| NC4/De500 | 186974_s_at | Y49F6B | Y49F6B.10 | COL-71 | + |
| NC4/De500 | 189762_at | K09H9 | K09H9.3 | COL-49 | + |
| | 2 C-type lectin (15genes) | | | | |
| De4/In500 | 180562_at | B0218 | B0218.8 | C-type lectin domain | + |
| De4/In500 | 192509_at | ZK666 | ZK666.6 | C-type lectin domain | − |
| In4/De500 | 188947_at | T09F5 | T09F5.9 | C-type lectin domain | + |
| All De | 189975_at | F08H9 | F08H9.5 | C-type lectin domain | + |
| All In | 184035_at | M02F4 | M02F4.7 | C-type lectin domain | + |
| All In | 185425_at | Y38E10A | Y38E10A.5 | C-type lectin domain | + |
| All In | 194067_at | F35C5 | F35C5.9 | C-type lectin domain | + |
| De4/NC500 | 193836_s_at | W04E12 | W04E12.8 | C-type lectin domain | + |
| NC4/In500 | 181946_at | C03H5 | C03H5.1 | C-type lectin domain | + |
| NC4/In500 | 194063_at | F35C5 | F35C5.7 | C-type lectin domain | − |
| NC4/In500 | 177059_at | Y46C8AL | Y46C8AL.5 | C-type lectin domain | − |
| NC4/In500 | 173090_s_at | F35C5 | F35C5.8 | C-type lectin domain | + |
| NC4/In500 | 182790_at | F10G2 | F10G2.3 | C-type lectin domatn | + |
| NC4/In500 | 192559_s_at | F35C5 | F35C5.8 | C-type lectin domain | + |
| NC4/In500 | 177187_at | Y46C8AR | Y46C8AR.1 | C-type lectin domain | − |
| | 3 ShTK domain | | | | |
| | (13genes) | | | | |
| De4/In500 | 178297_at | T24B8 | T24B8.5 | ShTK domain | − |
| De4/In500 | 183527_s_at | C14C6 | C14C6.5 | ShTK domain | − |
| All In | 180973_at | F49F1 | F49F1.6 | ShTK domain | + |
| All In | 182573_at | T05B4 | T05B4.3 | ShTK domain | − |
| De4/NC500 | 184662_at | Y46G5A | Y46G5A.29 | ShTK domain | + |
| De4/NC500 | 178017_s_at | F01D5 | F01D5.1 | ShTK domain | + |
| NC4/In500 | 180410_at | Y39G8B | Y39G8B.7 | ShTK domain | − |
| NC4/In500 | 180727_at | F49F1 | F49F1.5 | ShTK domain | − |
| NC4/In500 | 182712_at | C49G7 | C49G7.4 | ShTk domain | − |
| NC4/In500 | 183020_at | F48G7 | F48G7.8 | ShTK domain | − |
| NC4/In500 | 183526_at | C14C6 | C14C6.5 | ShTK domain | − |
| NC4/In500 | 183665_s_at | F48G7 | F48G7.5 | ShTK domain | − |
| NC4/In500 | 178128_at | F01D5 | F01D5.3 | ShTK domain | − |
| | 4 GST (7genes) | | | | |
| In4/De500 | 175993_at | C29E4 | C29E4.7 | GST domain | + |
| In4/Nc500 | 192956_at | F11G11 | F11G11.3 | GST-6 | + |
| NC4/In500 | 190899_at | F21H7 | F21H7.1 | GST-22 | + |
| NC4/In500 | 190959_s_at | F37B1 | F37B1.5 | GST-16 | + |
| NC4/In500 | 191541_at | C54D10 | C54D10.1 | GST domain | + |
| NC4/De500 | 190962_s_at | F37B1 | F37B1.8 | GST-19 | + |
| NC4/De500 | 191303_at | Y53F4B | Y53F4B.32 | GST-29 | + |
| | 5 UDP- | | | | |
| | glucuronosyltransferase | | | | |
| | domain(7genes) | | | | |
| All De | 191882_at | F47C10 | F47C10.6 | UDP-glucuronosyltransferase | + |
| | | | | domain | |
| All In | 190693_at | K08B4 | K08B4.3 | UDP-glucuronosyl and UDP- | + |
| | | | | glucosyl transferase domain | |
| All In | 191418_at | ZC443 | ZC443.6 | UDP-glucuronosyl and UDP- | + |
| | | | | glucosyl transferase domain | |
| In4/Nc500 | 191251_at | C33A12 | C33A12.6 | UDP-glucuronosyl and UDP- | + |
| | | | | glucosyl transferase domain | |
| Nc4/In500 | 181099_at | F54C1 | F54C1.1 | UDP-glucuronosyl and UDP- | + |
| | | | | glucosyl transferase domain | |
| Nc4/In500 | 191502_at | ZC443 | ZC443.5 | UDP-glucuronosyl and UDP- | + |
| | | | | glucosyl transferase domain | |
| Nc4/In500 | 191568_at | C13D9 | C13D9.9 | UDP-glucuronosyl and UDP- | + |
| | | | | glucosyl transferase domain | |
| | 6 Vit (4genes) | | | | |
| In4/De500 | 172134_x_at | F56B6 | F59D8.2 | Vit-4 | + |
| In4/De500 | 173411_s_at | K07H8 | K07H8.6a, K07H8.6b, K07H8.6c | Vit-6 | + |
| In4/De500 | 194239_x_at | F59D8 | F59D8.1 | Vit-3 | + |
| Nc4/De500 | 177065_at | K09F5 | K09F5.2 | Vit-1 | + |

| Inc. Dec or no | Affy ID | | | | Metal/Zinc | Nucleic | |
|---|---|---|---|---|---|---|---|
| change | Repeated genes | Knownhuman | TMDs | Transporters | finger | acid | Known |
| | 1 Collagen (27genes) | | | | | | |
| In4/De500 | 188335_at | COL4A5 | 1_N | | | | |
| In4/De500 | 188245_at | — | 1_N | | | | |
| In4/De500 | 184144_at | COL6A3 | | | | | |
| In4/De500 | 189911_s_at | — | 1_N | | | | |
| De4/NC500 | 183010_s_at | COl12A1, TTBK2 | | | | | |
| All De | 183666_at | COL3A1 | 1_N | | | | |
| All De | 188391_at | COL3A1 | 1_N | | | + | bli-2 |
| All De | 189482_s_at | — | 1_N | | | | |
| All De | 189864_s_at | COL4A5 | 1_N | | | | |
| All In | 190134_s_at | COL5A1 | 1_N | | | | |
| All In | 186383_at | COl12A1, TTBK2 | 1_N | | | | |
| All In | 177024_at | — | 1_N | | | + | |
| In4/NC500 | 173647_s_at | col1A1 | 1_N | | | | |
| In4/NC500 | 189561_at | COL3A1 | 1_N | | | | |
| In4/NC500 | 192163_at | — | 1_N | | | | col-19 |
| In4/NC500 | 188406_s_at | col3A1 | | | | | col-8 |
| In4/NC500 | 188456_at | col7A1 | 1_N | | | | |
| In4/NC500 | 190530_s_at | — | 1_N | | | | col-20 |
| In4/NC500 | 188747_at | COL4A3 | 1_N | | | | |
| NC4/In500 | 188589_at | COL13A1 | 1_N | | Transferrin bind | + | |
| NC4/De500 | 173632_s_at | COL4A5 | 1_N | | | | |
| NC4/De500 | 173650_s_at | col1A1 | 1_N | | | + | |
| NC4/De500 | 174152_s_at | COL4A5 | 1_N | | | | bli-1 - |
| | | | | | | | (BLIstered |
| | | | | | | | cuticle) |
| NC4/De500 | 174960_at | COL9A3 | 1_N | | | | |
| NC4/De500 | 186349_at | mucin-2 | 2 | | | | |
| NC4/De500 | 186974_s_at | COL3A1 | | | | | |
| NC4/De500 | 189762_at | COL5A1 | 1_N | | | + | |
| | 2 C-type lectin (15genes) | | | | | | |
| De4/In500 | 180562_at | — | | | | | |
| De4/In500 | 192509_at | — | | | | | |
| In4/De500 | 188947_at | — | 1_N | | | | |
| All De | 189975_at | — | | | | | |
| All In | 184035_at | — | | | | | |
| All In | 185425_at | — | | | | | |
| All In | 194067_at | — | | | | | |
| De4/NC500 | 193836_s_at | mannose receptor, | | | | | |
| | | C type 2 | | | | | |
| NC4/In500 | 181946_at | MMR | | | | | |
| NC4/In500 | 194063_at | — | | | | | |
| NC4/In500 | 177059_at | — | | | | | |
| NC4/In500 | 173090_s_at | C-type lectin | | | | | |
| NC4/In500 | 182790_at | — | | | | | |
| NC4/In500 | 192559_s_at | CLECSF6 | | | | | |
| NC4/In500 | 177187_at | — | | | | | |
| | 3 ShTK domain | | | | | | |
| | (13genes) | | | | | | |
| De4/In500 | 178297_at | — | | | zinc finger | | |
| De4/In500 | 183527_s_at | — | | | | | |
| All In | 180973_at | mucin-2 | | | heme | | |
| All In | 182573_at | — | | | | | |
| De4/NC500 | 184662_at | Notch 2 | | | Calcium bind | | |
| De4/NC500 | 178017_s_at | — | | | | | |
| NC4/In500 | 180410_at | — | | | | | |
| NC4/In500 | 180727_at | — | | | | | |
| NC4/In500 | 182712_at | — | | | | | |
| NC4/In500 | 183020_at | — | | | | | |
| NC4/In500 | 183526_at | — | | | | | |
| NC4/In500 | 183665_s_at | — | | | | | |
| NC4/In500 | 178128_at | — | | | | | |
| | 4 GST (7genes) | | | | | | |
| In4/De500 | 175993_at | — | | | | | |
| In4/Nc500 | 192956_at | — | | | | | |
| NC4/In500 | 190899_at | Prostaglandin-D | | | | | |
| | | synthase | | | | | |
| NC4/In500 | 190959_s_at | Prostaglandin-D | | | | | |
| | | synthase | | | | | |
| NC4/In500 | 191541_at | — | 1_N | | | | |
| NC4/De500 | 190962_s_at | Prostaglandin-D | | | | | |
| | | synthase | | | | | |
| NC4/De500 | 191303_at | | | | | | |
| | 5 UDP- | | | | | | |
| | glucuronosyltransferase | | | | | | |
| | domain(7genes) | | | | | | |
| All De | 191882_at | — | 1_C | | | | |
| All In | 190693_at | — | 2 | | | | |
| All In | 191418_at | — | 1_C | UDP transferase | | | |
| In4/Nc500 | 191251_at | UDP- | 1_C | | | | |
| | | glucuronosyltransferase | | | | | |
| | | 2A1 precursor | | | | | |
| Nc4/In500 | 181099_at | — | 1_C | | | | |
| Nc4/In500 | 191502_at | — | 1_C | | | | |
| Nc4/In500 | 191568_at | — | | | | | |
| | 6 Vit (4genes) | | | | | | |
| In4/De500 | 172134_x_at | Apolipoprotein B- | | lipid transport ptn | | | vit-4 |
| | | 100 | | | | | |
| In4/De500 | 173411_s_at | — | | lipid transport ptn | | | vit-6 |
| In4/De500 | 194239_x_at | Apolipoprotein B- | | | | | vit-3 |
| | | 100 | | | | | |
| Nc4/De500 | 177065_at | — | | lipid transport ptn | | | vit-1 - |
| | | | | | | | (VITellogenin |
| | | | | | | | structural genes |
| | | | | | | | (yolk protein |
| | | | | | | | genes)) |

In accordance with the present invention, it is preferred that the library or catalogue be of C. elegans and mutants and alleles thereof. Most preferably, the library or catalogue will contain mutants and alleles involving each or multiples of the listed genes below implicated in heme homeostasis in C. elegans. That is, a mutant may have one or more of these genes omitted for the purpose of modelling and evaluating the effect of such omission on heme homeostatis in C. elegans. Since greater than 70% of the structural genes of C. elegans are also found in mammals, particularly humans, the library or catalogue will provide a model system for the study of eukaryotic, particularly human, heme homestasis.

Thus, using the above listing of structural genes, mutants and alleles of C. elegans may be prepared and studied. Specifically, the library or catalog may contain any number of single or multiple mutants or allelic forms of C. elegans or merely the genome or relevant partial genome of each. For example, entire structural genes of the C. elegans genome may be omitted or double or multiple copies thereof may be inserted. For a detailed discussion of well-known cloning procedures and methodologies which may be used in accordance with the present invention, see Current Protocols in Molecular Biology, Edited by Harvard, Medical School (Wiley 1987) and A Practical Guide to Molecular Cloning, by B. Perbal 1984). See also “the C. elegans pMap at nema.cap.ed.ac.uk/Ceorhabiditis/C_elegans_genome/celeganspmap.html; and “C. elegans research techniques” at nema.cap.ed.ac.uk/Caenorhabditis/techniques.html.

Preferably, the library or catalog will contain either a partial or full complainant of mutants and alleles of the approximately 308 heme-regulated genes identified in the preceding table.

Irrespective of whether a partial or full compliment of mutants and alleles of C. elegans is prepared, the effects of the mutations or allelomorphs are evaluated by observing their effects on worm homeostasis of ⁵⁹Fe-heme as described above, for example. Thereby, the contribution of each of the 308 noted heme-related genes or groups thereof in the case of multigenetic control may be evaluated and used as a model for the eukaryotic, and particularly human, heme homestasis.

In order to yet further illustrate the present invention, reference will now be made to several Examples which are provided solely for purposes of illustration and are not intended to be limitative.

### EXAMPLE 3

Phenotypic Characterization C. elegans Mutants that are Disrupted in Heme Homeostatis.

Although the pathways for heme transport and trafficking in mammals are unknown, specific proteins and regulatory mechanisms have been described in bacteria and yeast that govern the acquisition of heme from the environment, including proteins that mediate heme insertion into cytochrome c. These studies provide evidence that cytotoxic molecule such as heme does not merely diffuse through lipid bilayers within cells, but is acviely assimilated. We, herein, provide a scheme for cellular heme homeostatis in eukaryotes whereby heme is translocated across biological membranes via specific transporters and subsequently trafficked to different cellular compartments by “heme chaperones” (FIG. 1A). Our studies with C. elegans suggest that this animal is unique because of its inability to make heme albeit requiring heme to survive. Thus, C. elegans provides an excellent eukaryotic paradigm to examine the mechanisms of heme assimilation (FIG. 1B).

The purpose of this experiment is to elucidate the genetic specification of nutritional heme metabolism in C. elegans, by charcterizing specific mutants isolated from a forward genetic screen with specific defects in heme homeostasis and assimilation. It is imperative to use an unbiased approach because it is highly plausible that heme transport molecules in animals are divergent at genetic level from known bacterial and the recently identified yeast heme-binding proteins as no known orthologous proteins exist in mammals. Most important to the success of this experiment is the now-well established procedures in our lab for the biochemical and cell biological delineation of heme metabolism in C. elegans that is employed for the genetic characterization of heme mutants. This allows for the elucidation of molecular mechanisms for heme homeostatis.

The unique aspect of the experiment is in using the axenic CeHR liquid medium with controlled amounts of heme. This strategy for genetic screening has never been reported for C. elegans and thus represents a significant advancement for future studies related in nutrient utilization in an animal model. Using a genetic screen and analyzing the F2 progeny, 13 mutants have already been identified. With this protocol, a comprehensive analysis of mutants with specific defects in heme homeostatis, some of which are depicted in Table II above may be undertaken. For comparison, we have also included potential mutants that could be obtained by screening for animals that survive under low heme (≦1 μM). These mutants may have genes or alleles that will complementary our existing set of mutants eg: increased function of a heme transporter.

The mutants are catalogued concurrently, before focusing attention to a particular class of mutant(s). Phenoclusters (class A, B and C) reclassified by conducting a battery of biochemical, cell biological, and histochemical studies with respect to heme-dependent pathways. These are enumerated below.

(a) Morphological Analysis: Detailed examination of worm morphology is performed using standard DIC/Nomarski microscopy. This criterion is essential during all stages of analysis because mutations within the same genetic pathway may have similar morphological phenotypes. For example, both the Ras and Wnt signaling pathways determine the vulval cell fates of the vulval precursor cells, and mutations in either pathway leads to defects in vulva development. We use transmission electron microscopy (TEM) to examine specific tissues and cell types at the ultrastructural level if any morphological defects are observed with the mutants. Because every cell fate and their lineages have been mapped in C. elegans, these techniques allow for the determination of cell-type specificity in heme homestasis.

(b) Metabolic Heme Labeling: We ascertain whether the mutation in heme homeostatic pathways results in a concomitant change in the intracellular heme levels of the animal. Metabolic studies with radiolabeled heme, ⁵⁹Fe-heme, are performed as described hereinafter. Briefly, 50 ml of glacial acetic acid is stirred under a constant flow of N2 at 60° C. followed by addition of 12 mg of protoporphyrin IX in pyridine for 30 mm. To this mixture, 0.85 μCi of FeCl3 (specific activity 35.77 mCi/mg, Perkin Elmer, Boston, Mass.) will be stirred-in for an additional 3 h. The incorporation of ⁵⁹Fe into PPIX is monitored spectrophotometrically and is complete when there is no further reduction in the absorbance of PPIX in pyridine at 408 nm. Heme is extracted from this mixture with ethyl acetate followed by extensive washes with 4 N HCI and distilled water to remove unincorporated PPIX and iron. The heme, thus obtained, is concentrated by evaporation of the ethyl acetate using a RotaVapor and frozen at −20° C. until further use. Total amount of ⁵⁹Fe-heme synthesized is measured using a Packard Gamma Counter (˜21% efficiency). The purity of heme is determined by thin layer chromatography using silica gel 60 matrix in an NH chamber with 2,6-lutidine/water solvent.

Our studies indicate that worms can degrade heme to obtain iron under iron deficiency and heme sufficiency. Because the radioisotope in heme is ⁵⁹Fe, we might obtain unclear results if degradation of heme and the release of iron were both to occur. To circumvent this problem, we will perform parallel experiments with ¹⁴C-heme. To obtain high specific activity of labeled porphyrin (˜10 Ci/mol compared to 0.12 Ci/mol with rabbit reticulocytes), ¹⁴C-heme is synthesized using the unicellular photosynthetic red algae, Cyanidium caldarium mutant strain III-D-2 which produces more porphyrin per cell than wild type. When grown in the dark, in minimal medium containing glucose and aminolevulinic acid (American Radiolabeled Chemicals, St. Louis), relatively large amounts of protoporphyrin IX are excreted into the surrounding medium. We obtained this C. caldarium strain from Dr. David Vernon at the University of Leeds, UK and synthesize ¹⁴C-heme from ¹⁴C-ALA by isolating and concentrating the ¹⁴C-protoporphyrin IX from the culture medium and then chemically inserting ferrous sulfate and purifying ¹⁴C-heme with ethyl acetate described. See also Rao, A. U. et al., Proc. Natl. Acad. Sci. USA 102, 4270-5 (2005).

Heme uptake and accumulation in cultured C. elegans is assayed by metabolic labeling with radiolabeled heme. Equal numbers of growth synchronized L1 larvae are inoculated in T25 flasks containing sterile CeHR medium containing 1.5, 4, 20 and 500 μM hemin. Worms are harvested at L4 or gravid adult stages prior to radiolabeling experiments. They are incubated with M9 buffer for 30 mins. for their intestinal contents to empty. Approximately 20,000 staged worms are plated in triplicate onto 24 well plates containing CeHR medium with no added hemin. Uptake assays are initiated by incubating 10⁵ cpm of radiolabeled heme for different time points at 20° C. by rotation. This method of direct metabolic labeling is more accurate and can be easily manipulated during kinetic analysis, compared to radiolabeling E. coli prior to feeding these bacteria to worms. Non-specific background is taken into account by performing a mock uptake with worms incubated with 1 mM sodium azide. If need be, heme uptake measurements are performed at timed intervals and multiple heme concentrations utilizing radiolabeled heme as a tracer. Accumulation studies are done by incubating each well of worms with 10⁵ cpm for multiple time-points with concentrations pre-determined from our kinetic analysis. Worms are collected, washed, lysed, analyzed on TLC, and measured with a gamma counter as described. As a positive control for metabolite uptake and to test the efficacy of inhibitor treatments, the energy-dependent transport of ³H-succinate, a dicarboxylic acid known to be transported by NaDC2 gene product in the worm intestine is measured. Total protein for all experiments is measured by the Bradford or bicinchoninic acid methods, and the data normalized to mol/mg of total protein or mol/number of worms as described. Prior to the start of each experiment worm viability and morphology are monitored using DIC microscopy. These measurements provide a quantitative analysis of specific defects in the transport and sequestration of heme in the mutants relative to each other.

Hemoprotein Activity: To measure hemoprotein activity as a function of organismic heme status, ultra-low temperature spectra is used for qualitatively determining cytochromes a, b, and c. We have standardized this methodology, and can easily observe discernable differences in cytochrome spectra as a function of exogenous nutritional heme levels. We also correlate the cytochrome levels with total heme analyzed by pyridine hemochromogen method. In addition to these experiments, we directly measure heme-enzyme activities spectrophotometrically by assaying cytochrome c oxidase, catalase, peroxidase, and cytochrome b5 reductase. By analyzing these specific enzymes, we plan to probe multiple sub-cellular compartments including the mitochondria, peroxisomes, lysosomes, secretory pathway, and the endoplasmic reticulum. Because worms have more than 80 CYP45O orthologs we do not assay for those enzymes. If there is a defect in heme trafficking pathways (hemochaprone) downstream of the heme transporter, we detect them by enzyme assays. Taken together, these studies provide a “picture” of heme trafficking, i.e., defects specific to a single class of hemoprotein(s).

Viability Assays: As noted above, we have determined that certain metal-ligand compounds, such as the heme analog GaPP is ˜800-3000 times more toxic than hemin to P0 and F1 animals. Data have suggested that such metal-ligand chelates like GaPP act as a Trojan horse and gains entry into cells via the heme transport system. Ga and Fe have very similar ionic radii, but unlike Fe, Ga does not undergo oxidation-reduction reaction. Thus, binding or inserting GaPP results in obstruction of heme trafficking pathways and inhibition of heme-dependent enzymes. We exploit these attributes of GaPP, for example, to probe mutants because, in principle, we are able to not only analyze mutants of heme import (these should be equivalently resistant to heme and GaPP toxicity) but also heme—trafficking/sequestration downstream from heme uptake (these mutants should be dissimilar in their toxicity to heme and GaPP). An example of the latter is mutant IH828 in class B (FIG. 12). This mutant is moderately resistant to 800 μM hemin but does not grow in 1000 μM hemin. Surprisingly, IH828 is extremely resistant to GaPP toxicity. This is in stark contrast to mutants IH731 and IH718 which represent class A and C, respectively. All three mutants have very different phenotypes, with no direct correlation between heme-resistance and GaPP toxicity. The experiment in FIG. 12 illustrates an important point—although our phenoclusters are primarily designed based on heme growth profiles, these could be further sub-clustered based on secondary criteria, described in this specific aim. Thus, careful analysis of each of the mutant from the phenoclusters allows one to dissect the differential preference for GaPP and provide clues to intracellular heme transport.

Fluorescent Imaging in Live Worms: We use live worm imaging with ZnMP to visually characterize the defects in heme transport using fluorescent microscopy. These studies provide detailed insights into the cell biological defects in heme pathways, i.e., decreased transport will result in lower fluorescence and aberrant trafficking and sequestration may reveal mislocalization of heme within cells or in a specific cell type such as the intestine, gonads or muscle. We have standardized this methodology in wild-type C. elegans with respect to ZnMP concentrations, incubation times, and measurements to accurately quantitate fluorescence intensity. More recently, we have performed experiments with ZnDP (Zinc deuteroporphyrin IX 2,4 bisethylene glycol), a highly fluorescent heme analog that is water soluble compared to the typical hydrophobic porphyrins, including ZnMP. These types of porphyrin compounds and even tetrapyrrole compounds are available by design from Frontier Scientific, Logan Utah.

In situ Heme Staining: We have extensively standardized the methodology for using DAB to visualize heme peroxidase staining in wild-type whole-mount animals. The current method was empirically derived and adapted to worms using a combination of several published protocols. The wild-type and mutant worms are incubated in a modified methanol and paraformaldehyde solution. These fixed worms are reduced using 10 mM DTT followed by incubation with 0.2% catalase and 0.02% superoxide dismutase (Sigma Chemicals). We have found that this treatment dramatically reduces staining in control samples because of endogenous oxygen radicals production. All solutions are degassed by bubbling nitrogen gas followed by vacuum suction. The animals are stained using 0.15% DAB and 0.2% H₂O₂. If any aberrant phenotype is observed in the mutants (differential intensity and atypical staining pattern) we simultaneously stain intact worms for cytochrome oxidase.

We support our histochemical observations in the mutants with electron microscopy. We use a PELCO BioWave 34700 microwave with the worm samples sitting in Pyrex well slides on an ice bath. The microwave energy helps to get the fixative solutions past the worm cuticle. The worms are fixed with paraformaldehyde and glutaraldehyde. The fixed worms are treated with solutions containing CAT/SOD followed by DAB/H202 staining as described above. The samples are destained in 0.2 M HEPES, pH 7.4 and treated with 0.1% osmium tetroxide for 2.5 h. Washed specimens are embedded in 2.5% SeaPlaque agarose, dehydrated through alcohols, and embedded into plastic resin for thin sectioning.

These studies are not sequential, but are performed simultaneously with the three potential phenoclusters of mutants. Of particular interest, are mutants (eg: IH828) that reveal an interesting phenotype with respect to heme entry into cells and subsequent sequestration, because these two steps are upstream of all subsequent pathways (heme insertion and trafficking to subcellular compartments). It is possible that loss-of-function mutation in an essential heme transporter may be embryonically lethal. However, point mutations, as in our EMS-based screening, in specific regions of a protein may result in decreased activity of the transporter/receptor due to diminished affinity for binding of heme or a secondary molecule involved in the pathway. As noted previously, we have phenoclustered our mutants into three separate classes, based on obvious growth phenotypes with respect to low and high heme levels. The combination of data obtained from the characterization of these heme mutants affords a classification of genes based on “phenotypic signatures”. Phenotypic clustering has already been applied in other studies with C. elegans and Drosophila. The “phenome” map resulting from this genome-wide analyses affords a detailed understanding of a variety of heme-dependent biological processes.

### EXAMPLE 4

Determining the Molecular Identities, of the Mutated Genes in C. elegans.

The objective of this experiment is to identify the molecular lesion in the mutants isolated from our forward genetic screen, and clone the corresponding genes responsible for the mutant phenotype. We also use parallel strategies to identify molecules involved in heme transport, not identified by our genetic screening using heme resistance, with a functional RNA interference (RNA1) approach using a reverse genetic screen. In addition, data obtained from establishing the phenotypic parameters for each class of mutants, affords a precise delineation of the molecular basis of the specific mutations of interest.

Genetic complementation analysis is also conducted in parallel with the experiments outlined in Example 3, because this allows us to rapidly determine if we have multiple genes within a phenocluster. Recessive mutants are crossed bringing together the genotypes in the F1 progeny. If that F1 individual is mutant, then the complementation has failed, and thus the two alleles are on the same gene. If no mutant phenotype is observed in the F1 individual, then the mutant alleles are complemented and must be different genes. Thus, the complementation test allows us to identify and sort animals with mutations within the same gene (allelic or intragenic) or different gene (non-allelic or intergenic). We use wild-type males to mate with homozygous mutant hermaphrodites (m1/m1) to obtain m1/+ heterozygous males. We do these by crossing 10 males with 2 hermaphrodites on 10 NGM agar plates spotted with E. coli. We first let the hermaphrodites exhaust her sperms by allowing her to lay eggs for two days and which point we will add the +/+ males. The resulting progeny is highly likely to be a cross-progeny as the males will provide the sperms of the fertilized eggs. To ensure cross-progeny we pick only F1 males (m1/+) and repeat the crosses with another mutant hermaphrodite (m2/m2) using the same techniques described above. From the resulting F2 progeny, eggs from the first 12 h are discarded as these are likely to have self-fertilized eggs. About 100 eggs are then collected from these crosses and allowed to hatch in M9 buffer (containing antibiotics) as this results in synchronization of the newly hatched L1 larvae due to nutrient deprivation. The L1s are then grown in CeHR medium with either 800 μM or 1000 μM hemin in the presence of antibiotics to prevent bacterial growth (50 μg/ml each of streptomycin, tetracycline, and nalidixic acid). If the L1s do not grow at high heme then m1 and m2 have complemented (m1+/+m2) each other and are non-allelic.

To determine whether two mutants, IH728 and IH731, belonging to the same phenocluster (class A) have mutations within the same gene we used genetic complementation as described above and found that mutations in IH728 and IH731 are most likely to be in two serarate genes. Although it is rare, two non-allelic mutants may fail to complement for example, if the two mutations are synthetically dominant negatives. Alternatively, allelic mutants may complement if the two alleles have mutations that counteract each other and restore wild-type functions.

Based on the number of complementation groups that are found, we correlate this finding with our three classes of mutants. It is likely that we may find multiple hits in the same gene in class C mutants because they represent the largest group amongst the three. Based upon this analysis, we are able to judge whether our mutant genetic screen is saturated and that we have identified all the genes that can result in resistance to heme toxicity. Based upon the nature of the complementation groups we then map and localize the mutations using current, standard techniques. We simultaneously pursue genetic mapping by restriction fragment length polymorphism in combination with single nucleotide polymorphisms (snip-SNPs) and by crossing into specific mapping strains that are available from the CGC.

Then, the mutation to one of the six chromosomes is mapped by using strains, MT465 [dpy-5(e61)l; bli(e768)ll; unc32(e189)Ill] and MT464 [unc-5(e53)IV; dpy-11(e224)V; Ion-2(e678)X]. Each strain has three successive homozygous recessive mutations or “markers” on each of the chromosome (I, II, III and IV, V, X) which results in a visible phenotype. To perform this experiment we use sperm-exhausted homozygous hermaphrodites (m/m) and mate them on agar plates to wild-type N2 males resulting in heterozygous F1 males (m/+). These males are then mated to the mapping strain, for example MT465 which has three phenotypes—dumpy, blister and uncoordinated. The resulting F2 progeny is normal heterozygous for all markers. F2 hermaphrodites are singled out on 12 individual plates and allowed to lay F3 progeny. Gravid F3s that are either dumpy, blister or uncoordinated homozygous are picked, pooled and transferred to liquid CeHR medium with 800 μM or 1000 μM hemin. Resistance to high heme is scored by observing growth in the F4 progeny. If any one of the three markers do not show heme resistance than our mutation is on that chromosome. However, in cases where the mutation is tightly linked to the genetic marker, expected segregation may not be observed. In that case we use a different marker strain easily obtained from CGC.

Using the methodology described above, we have mapped the mutation in IH1048 to chromosome lll. Using IH1048 as an example, we use three-factor mapping by mating IH1048 to BC4166 which has three mutations on chromosome lll [dpy-17(e164) let-747(s2456) unc-32(e189)111] and analyze the segregants. This allows us to map our mutation to an genetic interval on chromosome lll. We simultaneously confirm this chromosomal location by using snip-SNPs. For snip-SNPs we use the Hawaiian strain CB4856 which shows a high level of polymorphism across the genome compared to the wild-type N2 strain.

Mutant (m/m) hermaphrodites are crossed to CB4856 males (+1+) on agar plates and 12 F1 hermaphrodites from the cross progeny (m/+) are picked onto single plates. These m/+ animals are allowed to lay eggs for 36 h, and eggs from each plate are picked into microfuge tubes containing M9 buffer with antibiotics to ensure synchronization of the L1 larvae (F2) for later analysis. To be certain that the 12 m/+hermaphrodites are indeed cross progeny and not self, we analyze the genotype of each animal for a random marker by performing single worm PCR standardized in our lab. Only the progeny from a cross between N2 and CB4856 is assessed by “bulk-segregant analysis (BSA)” using snip-SNPs. FIG. 13 is an example of BSA using snip-SNPs and clearly shows that all of the necessary techniques and reagents available to perform high-resolution mappings are described herein.

The F2 synchronized L1 cross-progeny are singled out into humidified 96-well plates containing 100 μl of CeHR medium with antibiotics and either 800 μM or 1000 μM hemin. This selection allows us to identify mutant phenotype (m/m) from non-mutant animals (m/+ and +/+). We allow the F2 worms to grow until gravid adults are observed in the selection medium at which point 45-60 worms that grow and don't grow at high heme (BSA) are pooled. We these worms in buffer containing Proteinase K and perform PCR with 3 sets of paired primers per chromosome each corresponding to the left, right and central portion of a chromosome. Thus, we have 18 PCR reactions (3 reactions×6 chromosomes=18) per phenotype and 36 PCR reactions total (18×2=36) for any one mutant. The PCR reaction is then digested with restriction enzymes and analyzed on 2% agarose gels to estimate band intensity by automated image analysis using the BioRad ChemiDoc system. The ratio of the intensity for CB4856-specific and an N2-specific band for each of the mutant versus wild-type phenotype is calculated using the procedure described by Wicks et al., Nat. Genet. 28, 1500160-4 (2001). By using this technique, we mapped the mutation in IH1048 to the left arm of chromosome lll.

Repeated PCR analysis followed by digestions with restriction enzymes permits identification of the approximate location of mutant genes at high resolution. Information regarding the coordinates of all C. elegans SNPs are publicly available at http://genome.wustl.edu/projects/celegans. Because the genetic and physical maps of the C. elegans genome are well characterized, a gene affected by a chemically induced mutation is typically identified using a positional cloning approach that involves the following three phases. (a) High resolution map, the mutation is positioned on the physical map (70). This defines an interval that contains the gene. (b) transgenic animals containing genomic DNA from this interval cloned in cosmid or YAC vectors are generated, and assays for rescue of the mutant phenotype are conducted. This approach is used to search for a DNA fragment that contains the mutated gene and then to define a minimal rescuing fragment. (c) Candidate open reading frames (ORFs) are sequenced positioned on the minimal rescuing fragment using DNA from mutant animals to identify the nucleotide change that causes the mutant phenotype.

High-resolution mapping is useful and important because it significantly reduces the difficulty of subsequent cloning steps. Depending on the minimal interval that we map our mutation, we will use two approaches.

Firstly, if we identify the mutation to a small interval <15 kb, it is practical to identify the molecular lesion by DNA sequencing and bypass the need for the standard procedure of transformation of mutant worms with genomic DNA to identify a rescuing fragment. This is important because transformation can be laborious and is prone to both false-negative and false-positive results. Here obtaining a high-resolution map is particularly useful for positional cloning genes identified by mutations that cannot be rescued by injection of wild-type DNA, e.g. mutations that affect genes that function in the germ line, a tissue in which transformed genes are not expressed efficiently. Secondly, if we cannot locate the mutation to a manageable interval, we simultaneously perform deficiency complementation to determine a region of DNA that can rescue the mutant phenotype and mimic the mutant phenotype by “knock down” experiments using RNA interference (RNAi) by microinjection.

The entire C. elegans genome (>99%) is contained within 2527 cosmids (˜35 kb) and 257 Yeast Artificial Chromosomes or YACs (˜100 kb to 3 Mb), both available from Dr. A. Coulson of the Sanger Centre, Hinxton, UK. The use of two different host-vector systems allow us to get round problems of “unclonable” DNA—segments of the genome which can be propagated only poorly in one system but can be stable in the other. To test whether the predicted region of the genomic DNA comprises our mutation, we use a transformation rescue assay. The mutant animals are transformed by microinjection with cosmid or YAC DNA and a plasmid that contains a dominant rol-6 mutation as a transformation marker. At least six independently derived transgenic strains that displayed the Rol phenotype are obtained and their ability to survive heme toxicity is analyzed. Complete rescue is accomplished if the transformed mutants now show wild-type phenotype, i.e. 800 μM or 1000 μM hemin is toxic and result in growth arrest. We then analyze the functionally complementing fragment for all predicted ORFs and empirically determine if any predicted ORF is sufficient to rescue the heme-resistant phenotype by constructing plasmids with overlapping contigs and ORFs. If we do get genetic rescue we then use plasmids containing a deletion of predicted ORF as controls. A failure to rescue in the control indicates that the deletion in predicted ORF likely reduces gene activity, and supports the assessment for that ORF.

To pinpoint the exact nature of the molecular lesion in the mutants, multiple long-range PCR reactions are performed to amplify the gene using the Phusion High-Fidelity DNA Polymerase kit from MJ Research (BioRad) which amplifies >40 kb DNA with high fidelity. The agarose-gel PCR products are purified and the fragments are sequenced. We use a DNA core facility. If we locate the lesion in the PCR product, the ORF is determined by in silico analysis at the Wormbase website. The identified ORF is then vitro transcribed using T7 promoters flanked on either ends and the purified dsRNA is injected into wild-type worms to directly assess whether our mutant phenotype is comparable to the RNA1 phenotype. Further, phenotypic characterization of identified gene is performed by using the criteria listed in the previous Examples.

### EXAMPLE 5

Functional Characterization of Target Genes Identified From Global Gene Expression Profiling in Response to Heme.

The overall goal of this experiment is to understand how heme regulates gene expression as a function of nutrient availability and animal development using C. elegans as a model system of heme auxotrophy. A key part of elucidating the cellular role of heme is to determine the in vivo targets of this important cofactor. This question is addressed by characterizing target genes that we identified using Affymetrix Microarrays (GeneChip). The microarray approach was used because worms reveal regulated transport of heme (pulse-labeled with ZnMP) when grown under heme replete versus heme deplete conditions. Thus, as a first choice for identifying regulatory mechanisms, transcriptional profiling is appropriate because thus far only a few genes have been identified that are bona fide heme dependent targets in eukaryotes.

Microarray analysis has been successfully used in C. elegans for the identification of genes in germ cell development, signaling events, RNA interference pathway, and muscle development. Some of these data, including genes regulated during germ cell differentiation, are publicly available on the C. elegans webserver (www.wormbase.org) allowing us to selectively compare and categorically defer genes involved in these processes but no direct relevance to heme metabolism. For example, even though we used highly growth synchronized late L4 larve in our microarray experiments, it is possible that a small but significant number of worms progressed past this developmental stage to young adults. In this case, genes involved in gonadogenesis, oocyte and sperm development are induced.

Microarrays can reveal genes for global regulators and we are able to analyze those candidates in our initial group because this single regulator is the direct heme target but then regulates the expression of several downstream target genes that are indirect heme targets. An excellent example of this mode of regulation is Cth2 which was recently discovered in S. cerevisiae using DNA microarrays performed under low and high iron. Cth2 binds to the 3′ UTR of iron-responsive genes in response to iron deficiency and coordinates global metabolic reprogramming of >20 genes in response to iron. Cth2 target genes were discovered by repeating the microarray experiments and comparing the genes that are aberrantly expressed in Cth2 mutants compared to wild-type. See Puig, S. et al., Cell 120, 99-110 (2005).

By microarray, we have identified 280 genes, a large proportion of these having human orthologs. Our current microarray approach tells us which genes are differentially regulated by heme, but does not distinguish between direct and indirect targets. Because the total number of genes is only 280 or 1.35% of the worm genome, we are able to validate these genes. Microarrays tend to suppress changes in gene expression, and thus the gene expression must be collaborated by other methodologies. We validate genes based on a combination of the following criteria: (a) differences in fold-change with respect to low and high heme, (b) presence of heme and metal binding domains/motifs, (c) heme-responsive transcriptional regulators, (d) presence of potential transmembrane domains indicative of membrane transport functions.

The initial statistical analysis of the Affymetrix genome array was performed at the NIDDK microarray facility using Affymetrix MAS 5.0 Suite software. Of the 22,627 probe sets on the array, the MAS 5.0 algorithm revealed changes in 886 genes in response to heme, See FIG. 12. To further narrow-down candidate genes, we eliminated 58 genes that are involved in worm developmental processes by comparing the data from the gene chip to the microarray data of S. Kim. See Kim, S. K. et al., Science 293, 2087-2092 (2001). Of the remaining 828 genes, only 124 genes had a fold change of >2 (FIG. 12). Changing the threshold value between 1.6 and 2 revealed an additional 156 genes. These 280 genes were then sorted based on different categories of changes in gene expression (up, down or no change) seen in 4 μM versus 500 μM samples using 20 μM dataset as the baseline (Table XX). Protein sequence motif analysis (BLOCKS, PRINTS, Pfam), transmembrane (TM-HMM) and secondary structure predictions (ExPASy) proteomics tools are performed to identify domains and motifs within the predicted ORFs mRNA Ct value and the housekeeping gene Ct value: ΔCt=Ctₛₐₘₚₗₑ−Ct_{reference}. Relative mRNA abundance represents the difference between ΔCt values for a pair of conditions, i.e., 4 μM versus 20 pM. Relative mRNA expression is exponential and defined by the formula [Mrna]=2^{-ΔΔCt}. Thus, in our experiments we will use the following equation: 2^{-ΔΔCt}=[(Ctₛₐₘₚₗₑ−Ct_{reference})^{treated}−[(Ctₛₐₘₚₗₑ−Ct_{reference})^{untreated}], where sample: candidate gene, reference: housekeeping control, treated: 4 or 500 μM samples, untreated: 20 μM samples. The standard error for ΔΔCt will be calculated by the method of Livak and Schmittgen. See Livak, K. J. et al., Methods 25, 402-8 (2001).

An example of initial microarray data validation for R02E12.6 shows a 16-fold upregulation under low heme (4 μM) in microarrays and encodes for a putative permease transporter with four transmembrane domains and several “cytochrome-like” motifs. Validation of this gene by qRT-PCR and calculating fold-change using the 2-method revealed >40-fold upregulation under low heme and confirmed the expression pattern under different heme conditions. However, the 2^{-ΔΔCt} method is based on certain assumptions, such as equal amplification efficiencies for the target and reference genes which may not always correlate. Under such circumstances, it becomes imperative to include standard curves with every run to account for all possible sources of variation account for all possible sources of variation. The sensitivity of the qRT-PCR is a major advantage that allows it to be put to use for validation of data from microarrays although there may not be direct correlation between data from qRT-PCR and gene chips. Candidate genes validated by qRT-PCR are therefore be further confirmed using RNA (Northern) blot analysis using standard methodology available in our laboratory. Validation of expression profiles of the candidate genes using two different techniques allows for the obtainment of reliable and reproducible data.

Any genes identified by our four criteria, listed above, and validated by qRT-PCR and Northern blot analysis are most likely to represent target genes directly regulated by heme and may then be further characterized. Bona fide heme regulated genes will be “knocked down” using RNA interference (RNAi). A two-step PCR is conducted to generate templates for in vitro transcription using Ambion's T7 Megascript kit. The quality and integrity of dsRNA is evaluated by gel electrophoresis using RiboProbe (Molecular Probes) and the concentration is determined by spectrophotometry. Worms are subjected to double-stranded RNA by either injection into their gonadal arms using standard procedures or by soaking them in dsRNA. Alternatively, worms can be fed dsRNA using RNase Ill deficient E. coIl strain HT1 15(DE3). Continuous exposure to these bacteria allows for sustained assessment of the consequences of specific genetic interference. However, a major drawback from a nutritional viewpoint is the presence of heme replete E. coli which may confound data interpretation as heme levels are no longer defined. To circumvent this issue, we standardize a combination of RNAi feeding and concomitant growth in liquid culture. Results obtained are good with a control gene, pop-1, which causes embryonic lethality and is therefore easy to monitor success of our methodology. If any candidate genes cause maternal effect, embryonic lethal phenotype, thereby precluding the identification of a later heme-dependent phenotype, we perform an analysis termed “zygotic RNAi”. In this approach, RNAi-resistant rde-1/rde-I mutant hermaphrodites are injected with dsRNA then mated with wild-type males. F1 cross progeny are then examined for any zygotic phenotypes. These F1 progeny are saved from embryonic lethality because the dsRNA is ineffective in the rde-1/rde-1 mothers, however, if the dsRNA causes a zygotic phenotype these will be observed because the progeny are rde-1/+. See Herman, M. Development 128, 581-90 (2001).

Phenotypic characterization of the RNAi mutant animals is conducted essentially as described in Example 3 by analyzing (a) morphology using DIC microscopy, (b) heme dose-response growth curves to look for shifts in the biphasic pattern, (c) zinc mesoporphyrin fluorescence using fluorescence microscopy to determine which cell, where in the cell and at what developmental stage is the phenotype visible, (d) viability measurements with GaPP toxicity, (e) heme peroxidase staining using DAB and microscopy, and (f) hemoprotein activities. Genes identified by at least two of these approaches are analyzed in further details by transcriptional and translational fusions using reporter constructs.

Although there is a possibility that the RNAi might not reveal any overt phenotypes, the multi-faceted approaches for characterization allow us to identify such heme-dependent phenotypes. The efficiency of RNAi knock-down is usually about 80-95%. It is quite probable that sometimes residual amounts of the gene product is enough for activity, as observed in the Menkes Disease and Occipital Horn Syndrome. See Kaler, S. G. et al., Nat. Genet. 8, 195-202 (1994). In such an event, knock outs may be obtaied from deletion consortiums in C. elegans open to the worm research community (http://shigen.lab.nig.ac.jp/c.elegans and http://www.celeganskoconsortium.omrf.org/).

Green fluorescent protein (GFP) reporter constructs are generated by using a PCR fusion-based approach. For a transcriptional fusion, we use PCR to isolate DNA by amplifying ˜3 kb upstream of target genes. Most gene rescue and reporter gene experiments in C. elegans use only a few kilobases of upstream sequence and are successful, so for most genes this represents a good balance between promoter sequence length and PCR efficiency. The promoter region is fused with a fragment containing GFP and unc-54 3′UTR amplified from the vector pPD95.67 (Andy Fire lab). We analyze the expression of the GFP reporter at multiple developmental stages and in different cell types using fluorescent microscopy and confocal imaging. An important criteria is to analyze GFP expression as a function of heme concentrations. For example, from the microarray data, we expect that R02E12.6 promoter-GFP construct is turned on several fold under low heme but is at background levels at high heme. If the transcriptional reporter undergoes nonsense-mediated decay, we also inject this construct into animals in which this process is disrupted such as the smg mutants. See Hobert, O. Biotechniues 32, 728-730 (2002) and Cali, B. M. et al., Genetics 151, 605-16 (1999).

A translational construct will be generated by in-frame fusion of the open reading frame, including the 3 kb promoter region of specific candidate genes, to the amino and carboxyl terminus of GFP reporter. We clone the fusions into plasmid pPD95.75 which contain the 3′-UTR from unc-54. See Broday, L. et al. J. Cell Biol. 165, 857067 (2004). To generate extrachromosomal arrays, the fusion product is then injected into the gonads of young hermaphrodites, along with a marker gene such as rol-6 which helps to confirm that the DNA transformation experiments have worked from simply observing the movement of the animal. If necessary, stable transgenic lines are also constructed using biolistics, i.e. a “gene gun” by coating gold beads with our DNA construct and injecting this into cells by firing the particles into the worm at very high speeds.

A caveat associated with promoter-GPF reporter fusion is that it is helpful in understanding the spatial expression pattern of the gene but not necessarily temporal expression pattern. If degradation of GFP is not similar to the gene product of interest, a transcriptional reporter may only give information as to when the gene is turned on but not when it is turned off, i.e., when there is no active transcription of the gene. As an alternative, promoter-lacZ fusions can be made to compare the expression patterns of the GFP reporter. In situ hybridizations can be performed in parallel to study the mRNA localization. On the other hand, a GFP-translational fusion protein might result in GFP-related cell toxicity and therefore, might never be expressed at high levels. GFP may also cause mislocalization of the protein or disrupt the stability of the protein. To address this issue, the GFP tag can be attached to a different region within the protein (e.g., on the exoplasmic face of a TMD protein). As an alternative to GFP translational fusions, and if needed, immunofluorescence studies using an epitope tag such as with haemagglutinin (HA) are conducted.

Controlling and Treating Helminthic Infections in Mammals

In another aspect, the present invention provides a method for controlling and/or treating helminthic infections in mammals. Generally, any infectious parasitic nematode in a mammal, particularly a human, may be controlled and/or treated.

For example, the following infectious parasitic nematodes may be named: Ascaris suum, Trichuris suis, Haemonthus contortus, Strongyloides stercoralis, Ancyclostoma duodenale and/or Ancyclostoma species. However, these are only a few examples, and others are noted in the Table further below.

Generally, the method of controlling and/or treating a helminthic infection in a mammal entails administering an effective amount of one or more compounds as described below to a mammal in need thereof, which one or more compounds disrupt heme transport in a helminth, and having less, or no, disruptive effect on heme transport of the host mammal.

Any compound or mixture of compounds may be used to control and/or treat helminthic infections as long as the compound or mixture of compounds are able to disrupt helminthic heme transport, while having little or no toxicity in mammals.

An example of such compound or compounds are metal complexes of tetrapyrroles or porphyrins. Examples of such metals are gallium (Ga), tin (Sn), manganese (Mn), cobalt (Co), copper (Cu) and aluminum (Al), for example. Such metals have little or no known mammalian toxicity. However, other metals such as boron (B) and thallium (Tl) are not used in accordance with the present invention as they are toxic to mammals, and, thus, unsuitable. The compounds of the present invention are described in more detail below. One example of these comopunds, however, is gallium protoporphyrin IX.

### EXAMPLE 6

The effect of merely one exemplary compound, gallium protoporphyrin IX (GaPP), was tested on C. elegans to demonstrate the effect thereof against helminths in general and in treating mammalian parasitic helminthic infections.

Synchronized L1 larvae were grown in mCeHR medium supplemented with 4 μM hemin chloride and varying amounts of GaPP for 6 days. Worms were analyzed by DIC microscopy. Worms were grown in 2, 6, 8, 50, and 100 μM GaPP, respectively. Worms were also grown in mCeHR medium with 4 μM hemin. See FIG. 11, which shows the Toxicity of GaPP toward nematodes, even as compared to gallium compounds alone.

The ligand may be any tetrapyrrole or porphyrin-type compound, such as protoporhyrin, which includes a porphyrin ring nucleus.

The metal may be any metal which chelates or coordinates with tetrapyrroles or porphyrin-based compounds, and which is non-toxic to mammals, particularly humans, in amounts used to treat helminthic infections. Examples of such metals are gallium (Ga), vanadium (V), zinc (Zn), manganese (Mn), aluminum (Al), cobalt (Co), copper (Cu), tin (Sn) or even calcium (Ca) or magnesium (Mg).

Synthesis of Tetrapyrroles

Tetrapyrroles and porphyrin-based compounds are well known and commercially available. For example, custom designed tetrapyrroles and porphyrins may be obtained from Frontier Scientific in Logan, Utah. Their website is www.porphyrin.com. Further, synthetic strategies and methologies for preparing tetrapyrroles and porphyrin compounds are well known. See, for example, the work of Professor Smith and colleagues. www.chem.ucdavis.edu/groups/smith/Synth_Mech/Synth_Mech.html.

Notably, various oxygen-bearing side chains, such as -hydroxy, or -carboxy or even ethylene glycol groups may be used to enhance water-solubility of the ligands, if deemed necessary. The addition of such groups in tetrapyrrole and porphyrin synthesis is well-known as are methodologies for their synthesis.

Pyrroles and substituted pyrroles may be made by several methodologies. For example, the Paal-Knorr methodology may be used in which a 1,4-dicarbonyl compound or conjugated diyne is treated with ammonia or a primary amine. Successive nucleophilic addition and dehydration yields a pyrrole or substituted pyrrole, which is then further reacted to ultimately form a tetrapyrrole ring system. See also Schulte et al., Chem. Ber. 98, 88 (1965). Alternatively, using the Hantzsch synthesis, a β-keto ester is treated with an ∝-chloro-ketone in the presence of ammonia to ultimately yield a pyrrole or substituted pyrrole. See Principles of Organic Synthesis, R. O. C. Norman (Halstead Press, 1978).

Porphin, having the formula: 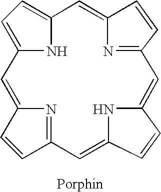
is the parent compound of the porphyrins. All of these compounds are prepared using known methodologies by first constructing four individual pyrrole rings, and then reacting two pyrroles in pairs to form two dipyrrylmethene compounds, and then joining the pairs.

There are at least three known methodologies for synthesizing dipyrrylmethenes.

First, a pyrrole-2-aldehyde is reacted with a second pyrrole possessing a free ∝-position in the presence of HBr. The acid increases the reactivity of the aldehydic group towards nucleophiles. Subsequent dehydration occurs readily.

Second, symmetrical dipyrrylmethenes may be produced by reacting a pyrrole with formic acid in the presence of HBR leading to successive reactions of a Friedel-Crafts type.

Third, a ∝-methylpyrrole containing a free 5-position is treated with bromine. A benzylic-type bromide is formed by one of the pyrrole units and this reacts at the 5-position of the second pyrrole in a Friedel-Crafts type reaction. See Principles of Organic Synthesis, R. O. C. Norman (Halstead Press, 1978).

Irrespective of the manner in which the dipyrrylmethene is produced, the coupling of two dipyrrylmethenes to yield a porphyrin compound is accomplished by heating a 2-methylderivative with a 2-bromo derivative in H₂SO₄ at about 220° C. Yields are usually no more than about 5%.

After obtaining thetetrapyrrole or porphyrin-type compound, the metal-ligand chelate may be obtained as described above with gallium protoporphyrin IX. See above. However, salts of other metals, such as Cu, Zn, Sn, Mn, Co, Mg or even Ca, may be used for example, instead of Ga, while otherwise using the same preparatory procedure. Typically, the chelate-complex is formed using any soluble salt of the metal such as the chloride.

The metal selected is relatively non-toxic to mammals, and preferably it has an ionic radii somewhat similar to that of Fe⁺³, i.e., generally, a difference of no more than about ±30% that of Fe⁺³. Also, it is of interest to use metals whose coordinating species is in their highest oxidation state in order to be non-participating in redox reactions. It is considered plausible that these characteristics lead to the observed cytotoxicity in helminths.

Examples of tetrapyrrole and porphyrin-based compounds which may be used as ligands are, for example, hydroxmethylbilane, uroporphyrinogen I, uroporphyrinogen III, coproporhyrinogen III, protoporphyrinogen IX and protoporphyrin IX.

Specific examples of the compounds of the present invention which may be used in the treatment of mammalian helminthic infections are: gallium protoporphyrin IX, vanadium protoporphyrin IX, manganese protoporphyrin IX, zinc portoporphyrin IX, aluminum protoporphyrin IX, calcium protoporphyrin IX, or magnesium protoporphyrin IX.

Other examples are protoporhyrin IX complexes with V, Zn, Mn, Co, Al, Ca or Mg. Similarly, complexes of either uroporhyrinogen I or III with any of Ga, V, Zn, Mn, Co, Al, Ca or Mg may be used. Further, complexes of coproporphyrinogen III or IX with any of Ga, V, Zn, Mn, Co, Al, Ca or Mg may be used.

However, it is emphasized that any compound having a porphyrin ring-type structure may be used as the ligand in the metal-ligand chelate complex. As used herein, the term “porphyrin ring type ring structure” means any compound having at least the porphyin ring structure noted in the above formula. The ligand compound may have more structural components such as ring substituents on any and all rings, isotopic substitutions on the rings or in the substitutents or both.

Further, it is understood that as used herein the term “tetrapyrrole compound” means all tetrapyrrole-based ligands, including, for example, tetrapyrrole, itself, as well as various substituted tetrapyrroles having one or more lower alkyl groups, carboxylic acid group, or lower alkyl carboxylic acid groups for example.

It is preferred, however, that a gallium complex of any of the ligands noted above be used. It is particularly preferred that a complex of gallium-protoporphyrin IX be used.

Further, it is also within the scope of the present invention to prepare various isotopic versions of the above complexes for metabolic studies with mammals as another manner of modelling human heme homeostatis. For example, any or more hydrogen atoms on any of these complexes may be replaced by deuterium or tritium. Also, any one or more nitrogen atoms in pyrrole rings of ligand may be replaced with nitrogen −15 (¹⁵N). Similarly, any one or more carbon atoms in the pyrrole rings of the ligand may be replaced with carbon −13 or 14 (¹³C or ¹⁴C). Any desired isotype may be incorporated into the starting materials using any of the known preparatory reactions noted with appropriate isotopic substitutions. See, also Synthesis & Applications of Isotopically-Labelled Compounds, Pleiss, U. et. al. (Wiley 2001).

The metal-ligand chelate complex of the present invention, may be administered directly as a powder or as a tablet to a mammal, particularly a human, in treating helminthic infections. Generally, an amount of the metal-ligand chelate complex administered to the mammal is in the range of about 1 mg to 500 mg per dose. The dosage regimen usually entails one administered dose per week. However, if desired or deemed necessary by a treating veterinarian or physician, more than one dose per week may be administered.

Alternatively, the metal-ligand chelate complex may be coated in the form of a tablet or capsule, which coating may be an enteric coating. Such coatings are well known.

Further, in formulating the dosage, whether in table, pill or capsule form, the metal-ligand chelate complex of the present invention may be mixed with any pharmaceutical—or veterinary—carrier or excipient, such as starch, lactose, magnesium stearate, for example. Generally, the metal-ligand chelate compound is present in an amount of from 1 to 99% by wt. of the total composition with the balance being a carrier or excipient. Any conventional pharmaceutical or veterinary carrier may be used.

As noted above, the metal-ligand chelate complex compounds are used in the teratment of helminthic infections in mammals, particularly humans. While any helminthic infection may be so treated, exemplary diseases and their causative aperts which may be so treated are:

| | |
|---|---|
| Helminth | Disease |

Cestodes:

| | |
|---|---|
| Echinococcus | Echinococcosis, Hydatid disease |
| Taenia solium | Taeniasis |
| Cysticercus (larva) | Cysticercosis |

Nematodes:

| | |
|---|---|
| Angiostrongylus | Eosinophilic meningitis |
| Strongyloides | Strongyloidiasis |
| Toxocara | Visceral larva migrans |
| Trichinella | Trichinosis |

Trematodes:

| | |
|---|---|
| Schistosoma | Schistosomiasis |

Generally, a treating physician or veterinarian will diagnose the condition of helminthic infection, and also monitor the progress of treatment.

Treatment of Helminthic Infections in Plants

The metal-ligand chelate compounds of the present invention may also be used in the prevention of and treatment of helminthic infections in plants.

Helminthic plant infections are a drain on agriculture throughout the world. Generally, nematodes such as, for example Trichodorus christei, feed on root epidermal cells of plants causing plant damage or even plant death. However, nematodes are also problematic as they are viral vectors. In fact, several widespread and important viruses in two viral groups are transmitted though the soil by nematodes. For example, member of the Nepovirus group are transmitted by species in the genera Xiphinema and Longidorus. Also, members of the Tobravirus group are transmitted by species of Trichodorus. See Fundamentals of Plant Virology, R. E. F. Matthews Accademic Press, 1992). It has been estimated that the soybean cyst nematode (SCN), for example, causes annual losses of over 250 million dollars in the U.S. Nematodes are known to cause extensive damage to plants as diverse as tobacco, strawberries, potatoes and corn. See www.ncagr.com/agrinomi/nemhome.htm.

The present invention provides metal-ligand chelate compounds as described above which may be added to the soil in the vicinity of the plant, i.e. at the base of the plant or on in the soil out to a distance of a few feet from the base of the plant. Generally, the present metal-ligand compounds may be added in the amount of about 10 mg to about 500 mg per square foot of soil Further, these compounds are typically mixed with a suitable inert carrier, such as sawdust or pulverized stone or clay, or they may be mixed with any conventional fertilizer composition. They also can, if desired, be mixed with conventional insecticidal compositions.

Furthermore, the metal-ligand chelate complex compounds of the present invention may be prepared so as to have sufficient water-solubility to be absorbed by the plant being treated. This approach provides a second line of defense against nematodes that survive soil treatment. To enhance water-solubility of the compounds, one or more hydrophilic side chains may be utilized on one or more of the pyrrole rings of the metal-ligand chelate. Exemplary hydrophilic groups are -hydroxy, -carboxy, -carboxyester (methyl- and ethylester), ether (methyl- or ethyl ether) or even ethyleneglycol groups. If the compounds are prepared to be water-soluble, they may be added to the soil adjacent to the plant being treated as a water solution with a concentration of the metal-ligand chelate complex therein of from about 0.001% to 1% by weight.

In the treatment of large areas of plants, such as large commercial forms, the compounds of the present invention may be sprayed in water solution from a tractor or from an airplane, for example, at low altitude. If a solid formulation is applied, it is preferably applied by tractor equipped with a spreader.

Generally, the present compounds may be used in the tretament of any plant helminthic infection or in the prevention of it. Examples of some diseases which may be prevented and/or treated by the present invention are root knot-caused by Meloidogyne spp., and other helminths causing diseases other than root knot, such as the nematodes Helicotylenchus spp., Hoploliamus spp., Heterodra spp., Globodera spp., Trichodorces spp., Longidoras spp., Belonolianus spp., Rotylenchus spp., Paratylenchus spp., Punctodera spp. and Paratrichodorces spp.

Plants affected by such helminths usually manifest chlorosis or slower than normal growth or even wilting under stress. Generally, plants affected by helminths are more susceptible to other unfavorable environmental circumstances, such as drought. These types of indicia may be used to consider whether helminthic infestation is a problem.

Further, the presence of nematodes, for example, may be determined by soil assay. This is often important inasmuch as growers frequently attribute nemadode-related growth reductions to nutrient or water deficiencies. Generally, agronomic experts advise that nematode problems cannot be identified solely on the basis of plant symptoms and that nematode assays are essential to a diagnosis of infestation.

For examples of soil sampling procedures and how to have samples properly analyzed for the presence of nematodes, see www.ppws.vt.edu/˜clinic (Virginia Tech), and www.dddi.org/uga/ppath/nematode.pdf (University of Georgia). Of course, such sampling and analysis may also be routinely conducted to monitor the progress of treatment.

Evaluating Heme Homeostasis in C. elegans and Eukaryotic Heme Homeostatis

In accordance with yet another aspect of the present invention, a method is provided for evaluating heme homeostasis in C. elegans by screening and classifying mutants that exhibit heme-dependent defects in normal growth and development. The mutants are characterized biochemically, and the mutations are then mapped and localized by genetic recombination and mapping of single nucleotide polymorphisms (SNPs). This is used to develop a model for eukaryotic heme homeostasis.

More specifically, the mechanism for defining heme acquisition in C. elegans is determined by: 1) measuring heme uptake into C. elegans grown in axenic medium following metabolic labelling with radiolabeled ⁵⁹Fe-heme, 2) analyzing heme incorporation into hemoproteins in C. elegans utilizing histochemistry and pulse-chase with ⁵⁹Fe-heme metabolic labelling, and 3) morphologically and biochemically analyzing live C. elegans by visually tracking heme transport and trafficking utilizing fluorescene microscopy with fluorescent heme analog, Zn- and Sn-substituted porphyrins, for example.

Further, the identification and characterization of mutants of C. elegans with disruption of heme homestasis is effected by: 1) generating and screening for C. elegans mutants that reveal normal growth and reproduction under sub-optimal levels of heme that are detrimental to wild-type worms, 2) categorizing these mutants into complimentation groups based upon their heme requirements and sensitivity to heme or heme analogs, and 3) mapping and localizing these mutations using genetic linkage by recombination and analysis of SNPs.

⁵⁹Fe is commercially available as are any of the isotopes noted above. Further, these isotopes may be incorporated into various metal-ligand coordination compounds by well-known synthetic methodologies.

The information obtained from this methodology is then used to elucidate eukaryotic heme transport. Specifically, by utilizing sequence homology and functional complimentation studies, the various contributions of receptors, permeases and ATP ases, for example, to eukaryotic heme transport are evaluated. The present invention also specifically contemplates a method of identifying eukaryotic heme transporters, as well as a method of modelling eukaryotic heme homeostasis.

What is claimed is:

1. An isolated parasitic helminth, which is a heme auxotroph.

2. The isolated parasitic helminth of claim 1, which is a nematode.

3. A nematode of the genus Caenarhabditis, which is a heme auxotroph.

4. The nematode of claim 3, which is C. elegans.

5. A catalogue, comprising mutants and alleles of C. elegans having specific and different genetic defects affecting heme homestatis comprising heme uptake, heme trafficking or heme incorporation or a combination thereof.

6. The catalogue of claim 5, which comprises mutants containing genetic defects in all or a portion of the genes affecting said heme homeostasis.

7. A method of modelling eukaryotic heme homeostasis, which comprises the steps of:
a) evaluating effects of the alleles and mutants of claim 5 on C. elegans heme homeostasis, and
b) producing a eukaryotic model of heme homeostasis therefrom.

8. The method of claim 7, wherein said eukaryotic model is a human model.

9. The method of claim 7, further comprising, after step b), identifying a eukaryotic heme transporter.

10. A method of evaluating heme transport in a parasitic helminth, which comprises the step of:
a) evaluating effects of the alleles and mutants of claim 5, on said heme transport, and
b) developing a model therefrom.

11. A method of treating a helminthic infection in a mammal, which comprises a step of administering one or more compounds which disrupt helminthic, heme transport to a mammal in need thereof.

12. The method of claim 11, wherein said one or more compounds are metal-ligand chelate compounds comprising a metal and a tetrapyrrole compound or a porphyrin compound.

13. The method of claim 12, wherein the metal comprises Ga, V, Mn, Cu, Co, Sn, Ca or Mg.

14. The method of claim 13, wherein the compound is gallium protoporphyin IX.

15. A method of treating a plant in order to prevent helminthic infection, which comprises adding one or more compounds which disrupt helminthic heme transport, to soil proximate to said plant.

16. The method of claim 15, wherein said one or more compounds are in a form of a composition.

17. The method of claim 15, wherein said one or more compounds are metal-ligand chelate compounds comprising a metal and a tetrapyrrole compound or a porphyin compound.

18. The method of claim 17, whrein the metal comprises Ga, V, Mn Cu, Co, Sn, Ca or Mg.

19. The method of claim 18, wherein the compound is gallium protoporphyin IX.

20. A composition comprising:
a) a metal-ligand chelate compound, wherein said ligand is tetrapyrrole-based or porphyrin-based compound; and
b) an inert carrier.
